(19) United States
(12) Patent Application Publication
(10) Pub. No.: US 2025/0057781 A1
(43) Pub. Date: Feb. 20, 2025

(54) ENGINEERED EXTRACELLULAR VESICLES WITH IMPROVED CARGO DELIVERY

(71) Applicant: Evox Therapeutics Limited, Oxford, Oxfordshire (GB)

(72) Inventors: Xiuming LIANG, Huddinge (SE); Dhanu GUPTA; Samir EL-ANDALOUSSI, Huddinge (SE); Joel NORDIN, Huddinge (SE)

(21) Appl. No.: 18/723,460

(22) PCT Filed: Dec. 2, 2022
(86) PCT No.: PCT/EP22/84310

§ 371 Date: Jun. 23, 2024

(30) Foreign Application Priority Data
Dec. 23, 2021 (GB) 2118946.9

(51) Int. Cl. A61K 9/51; A61K 38/46; A61P 35/00; C07K 14/005; G01N 33/50

(52) U.S. Cl. CPC A61K 9/5184; A61K 38/465; A61P 35/00; C07K 14/005; G01N 33/5005; C12N 2760/20222

(57) ABSTRACT

An engineered extracellular vesicle (EV) for delivering a bioactive cargo, the engineered EV comprising an endosomal escape enhancer and a cargo. Plasmids, polynucleotide sequences, cells, methods of making and analysis, pharmaceutical compositions and uses thereof.

### TECHNICAL FIELD

The present disclosure relates to engineered extracellular vesicles (EVs) with more efficient intracellular delivery, methods of making said EVs, as well as pharmaceutical compositions thereof and their use.

### BACKGROUND ART

Several techniques exist for intracellular delivery of proteins, such as nanoparticle polymer and lipid-based carriers. These different carrier types all aim to deliver cargo into a cell to drive modulation of intracellular proteins, genome editing, alter cell behaviours and inhibit certain interactions.

Engineering an exosome with the envelope protein Vesicular Stomatitis Virus Glycoprotein (VSV-G) is known from US Patent Publication Number U.S. Pat. No. 10,758,486B2 (Univ. Santa Clara) to produce gesicles for functional delivery of tetracycline-controlled transactivator (tTA) proteins into reporter cells. Minimal VSV-G is generally known from US Patent Publication Number US2020306297A1 (Univ. Santa Clara) for the investigation of EV biogenesis, drug loading and surface targeting, but not for functional intracellular delivery of cargos. The VSV-G, optionally fused to targeting molecules, is intended to increase interaction of the exosome with surface antigens on a target cell plasma membrane and the selective targeting of specific tumour(s) or tissue(s). In the aforementioned Santa Clara patent filings, the VSV-G has a role in targeting the exosomes to cells.

One disadvantage is that large volumes of EVs are required to get a high enough efficiency, such as the delivery into the cell.

As such, several technologies directed towards the delivery of cargo into the cytoplasm of the cell have been explored.

For example, Zhang et al., Developmental Cell (December 2020) Vol 55 issue 6 teaches an EV containing a specific cargo, preferably enzymes, RNAi, and Cas9 ribonucleoproteins (RNPs), tethered to VSV-G via a split green fluorescent protein (GFP) (referred to as gectosomes).

Disadvantages may include that the release of cargos is inefficient. As release of the cargo is difficult a higher dose of EVs will be required to achieve delivery of an effective amount of the cargo into the cytosol of a cell.

Photo-activated release systems have also been explored. For example, International Patent Publication Number WO2016/178532 (KOREA ADVANCED INST SCI & TECH) describes a dimeric optogenetic release system (CRY2-CIBN) for release of a protein cargo. Upon exogenously applied light exposure, two different optogenetic proteins associate and upon ceased light exposure the proteins dissociate. Cellex Life Sciences also has a system (EXPLORs) for engineering an exosome comprising optically reversible protein-protein interactions (CRY2-CIBN).

One disadvantage of a dimeric optogenetic release system is that it requires extended long-term exposure of biological material to a light source for transport of the protein into exosomes to occur and subsequently to release it from its exosomal transporter.

Another disadvantage is that it suffers from problems with scalability as it is highly cumbersome to carry out. In part this is due to the long-term exogenous application of light and the need for an external blue light initiator and an operator to turn on/off the blue light initiator.

A further disadvantage is that the prolonged exposure to blue light raises potential toxicity concerns and potential damage to cells. Such potential toxicity and cell damage is negatively viewed by regulatory authorities such as the FDA/EMEA meaning that any exosomes produced by such a method would also not realistically make it into the clinic and be FDA/EMEA approved. Consequently, in practical terms there remains a need to provide a therapeutic EV.

Further disadvantages also include lack of efficient delivery as it was found that the desired result could not be effectively replicated. The system was found not to work at least not without excessive levels of EV populations-further pointing to an impractical product with a lack of clinical relevance.

Another approach described in Kaczmarczyk et al., PNAS (October 2011) Vol 108, No. 41 employs virus-like particles (VLPs) containing a fusion protein of Gag and a protein of interest (Pol) (e.g., Pol is Cre or GFP) and a VSV-G envelope as co-transfected. International Patent Publication Number WO2020/252455A1 (MASSACHUSETTS GEN HOSPITAL) also describes a similar human-derived VLP (heVLP) with Gag and Pol approach.

Disadvantages of this approach include that release of the Pol from Gag is difficult due to the Pol forming a fusion protein with Gag. Certainly, release of the Pol in a free unconjugated form is unlikely meaning that the Pol will not be functional/active. As release of the Pol is difficult a higher dose of EVs will be required to achieve an effective delivery of the Pol into the cytosol of a cell. Gag is a membrane bound protein due to N terminal myristoylation. Disadvantages to using membrane associated proteins is that they tend to disassociate with the EV membrane, and it is unknown which part of the membrane the Gag would associate with (e.g., internal envelope; external envelope) meaning consistent loading is not likely. Protease sites such as murine leukaemia virus (MLV) or human immunodeficiency virus (HIV), in addition similar systems like idemr are thus typically used.

Commercially available technology developed by TaKaRa uses a cell-derived nanoparticle (gesicles) comprising an Inducible Heterodimer System (iDimerize™). The system enables control of the heterodimerization of two different proteins of interest in live cells via a membrane-permeable compound (or ‘dimerizer’) having two separate motifs. Delivery of a temporary dose of Pol is made possible without needing a plasmid. Apart from VSV-G, additional dimerizer ligand needs to be added into the cells when producing EVs, bridging the connection of Pol with the EV protein.

One disadvantage of the iDimerize™ technology is that it is very expensive. Another disadvantage of this technology is that the release of cargo is not efficient, needing extremely high doses of EVs to get decent recombination in reporter cells.

Further disadvantages may include that no data confirming co-localization of VSV-G with the EV protein are given by TakaRa. Lack of co-localization means that functional delivery is not practically taught.

Additionally, TaKaRa advocates treating cells with a high dose of rapamycin (an autophagy inducer), which is thought to affect the cellular pathway. There is also a huge chance that remaining rapamycin is then co-purified with the EVs.

International Patent Publication Number WO2020/099682 (EVOX Therapeutics) describes EVs that may potentially comprise various additional moieties to enhance bioactive delivery of cargo. Additional moieties, such as multimerization domains, release domains, and endosomal escape domains are described.

In brief, while various components are known to support bioactive cargo delivery by various nanoparticles including EVs they are not without their drawbacks. Use of certain components may give rise to potential immunogenicity and regulatory problems. Where more than one plasmid is required, double-stable cells are often problematic. Historically double-stable cell lines are often found to lose expression of one or both constructs over time. Additionally, stability of the cells and/or resulting EVs is a concern as errors in translation and protein folding etc., can lead to complications such as increased toxicity and/or lack of functional proteins. Importantly, where more than one construct might be used there is no guarantee that the constructs will find their way into the same EV, meaning that the full breadth of functionality would be lost. Engineering of EVs (e.g., exosomes) and of constructs, particularly fusion constructs that may be contained therein, is a skillful and nuanced art. As such modifications that may be necessary, for example, to fuse two proteins together may have an adverse and undesirable effect on the functionality and/or stability of the construct and/or EV (such as an exosome).

There remains a need for engineered EVs (such as exosomes) suitable for delivery of bioactive cargo.

One rate limiting step for each of the abovementioned techniques is the endosomal escape of said protein cargo and its release, in a substantially free unconjugated, and thus, functionally active form, into for example, the cytosol of a cell. Therefore, there remains a need for engineered EVs capable of endosomal release and the achievement of full delivery of a bioactive cargo at the desired location, such as the cytoplasm of a cell.

Another rate limiting step for the abovementioned techniques is the stability, or lack thereof, of constructs and inclusion of multiple constructs within the same nanoparticle or EV (such as an exosome). Therefore, there remains a need for engineered EVs and engineered cell lines that are capable of stably expressing constructs within the same EV (such as an exosome) as well as plasmids, and methods in support of the above.

Efficient delivery of cargo, particularly at lower doses, also remains to be solved.

### SUMMARY OF INVENTION

One object of the disclosure is to overcome at least one of the abovementioned problems associated with exosome mediated cargo delivery known in the art. Provided herein are engineered EVs having improved delivery of cargo.

In a first aspect of the disclosure there is provided an engineered EV for delivering a bioactive cargo. The engineered EV comprises an endosomal escape enhancer, and a cargo. The engineered EV may also comprise a release system and, if so, the release system may be a self-cleaving protein, such as an intein or any derivative, domain, variant, mutant or region thereof.

The intein may be a mini-intein, such as a mini-intein that has been modified to optimise the cleavage rate or delta-intein-CM. In one preferred embodiment the engineered EV also comprises an EV protein. In an alternative embodiment, the engineered EV may comprise a multimerization domain, such as a trimerization domain like foldon. The endosomal escape enhancer may be selected from VSV-G, cocal virus Glycoprotein (CVG), Prototype Foamy Virus (PFV) Envelope and heVLPs. The EV protein may be selected from an EV transmembrane protein or EV membrane associated protein. Where the EV protein is a transmembrane protein, it may be either a single-pass transmembrane protein or a multi-pass transmembrane protein, such as a tetraspanin like CD63, CD9, CD81 or a derivative, domain, variant, mutant or region thereof. The cargo may include a protein, enzyme, clustered regularly interspaced short palindromic repeats (CRISPR) protein, such as Cas9 and/or a nucleic acid binding protein. The EV protein may form a fusion protein with the cargo and, where present, the release system.

In a second aspect of the disclosure there is provided at least two plasmids, each comprising a polynucleotide construct. One polynucleotide construct encodes a protein construct that comprises an endosomal escape enhancer (e.g., VSV-G) and the other polynucleotide construct encodes a protein construct comprising a cargo, an EV protein and optionally a release system, preferably a self-cleaving protein such as intein.

Alternatively, there is provided one plasmid comprising a polynucleotide construct that encodes a protein construct, the protein construct comprising an endosomal escape enhancer such as VSV-G, a multimerization domain (such as Foldon) and optionally a release system (preferably a self-cleaving protein such as intein).

In a third aspect of the disclosure there is provided a cell comprising the plasmid(s) according to the second aspect.

In a fourth aspect of the disclosure there is provided a protein construct, for use in a nanoparticle (such as an EV, preferably an exosome). The protein construct comprises an endosomal escape enhancer such as VSV-G, a multimerization domain (such as Foldon) and optionally a release system (preferably a self-cleaving protein such as intein).

In a fifth aspect of the disclosure there is provided a method of making an engineered EV according to the first aspect, wherein the method comprises the steps of: (i) introducing into an EV-producing cell a polynucleotide construct encoding an endosomal escape enhancer, (ii) expressing the polynucleotide construct in the EV-producing cell, and (iii) loading a cargo into the EV, thereby generating an EV comprising both an endosomal escape enhancer and a cargo.

Preferably, the endosomal escape enhancer is displayed on the surface of the EV and the cargo is loaded into the lumen of the EV.

In a sixth aspect of the disclosure there is provided an in vitro method for assaying engineered EVs according to the first aspect of the disclosure. The method comprises the steps of: (i) co-culturing reporter cells and EV-producing cells capable of producing the engineered EVs, and (ii) measuring signal-positive cells. The reporter cells may comprise an above average receptor level expression, preferably VSV-G (LDL-R) receptor level expression. The above average receptor level expression may be from at least about 51%, and preferably at least 55%, of the overall expression profile. The EV-producing cells preferably include HEK293T cells. The method may have a delivery saturation between a cell-to-cell ratio of from about 1:5 to about 1:1 EV-producing cells to reporter cells. The method may have a detection limitation between a cell-to-cell ratio of from at least about 30:1, preferably from about 30:1 to about 50:1 and more preferably from about 30:1 to about 50:1. Most preferably the detection limitation is from at least about 50:1 when the reporter cell is B16F10.

In a seventh aspect of the disclosure there is provided a composition comprising an engineered EV according to the first aspect and an excipient, diluent, vehicle, solvent and/or carrier.

In an eighth aspect of the disclosure there is provided either an engineered EV according to the first aspect, or a composition according to the seventh aspect for use as a medicament, such as the treatment of cancer and/or brain-related conditions, disorders and/or diseases.

### BRIEF DESCRIPTION OF FIGURES

The disclosure will be more clearly understood by reference to the accompanying Figures, in which:

FIG. 1A shows the general domains and their arrangement to one another according to different embodiments of the present disclosure.

FIG. 1B shows a schematic illustration of protein constructs investigated according to different embodiments of the present disclosure.

FIG. 2 shows a pictorial representation of one method of preparing and testing EVs according to one aspect of the present disclosure.

FIG. 3 shows a pictorial representation of an alternative method to that shown in FIG. 2.

FIG. 4 shows a series of graphs illustrating the system parameters according to certain aspects of the present disclosure.

FIG. 5 shows a series of graphs illustrating the advantageous combination of features according to certain embodiments of the disclosure.

FIG. 6A shows a series of graphs illustrating the diversity of components that function according to the specific embodiments of FIG. 5.

FIG. 6B shows a series of graphs illustrating the diversity in different types of EV proteins that may be employed according to certain aspects of the present disclosure

FIG. 7 shows a set of Confocal microscopy images and corresponding graphs confirming functionality according to certain aspects of the present disclosure.

FIG. 8 shows a fluorescence activated cell sorting (FACS) plot confirming functionality and stability according to certain aspects of the present disclosure.

FIG. 9 shows a graphical representation of certain aspects of FIG. 8.

FIG. 10 shows a series of graphs illustrating the advantageous combination of features according to certain embodiments of the disclosure.

FIG. 11 shows a series of graphs illustrating the diversity of components that function according to the specific embodiments of FIG. 10.

FIG. 12 shows a graphical representation of the endosomal escape profile according to certain aspects of the present disclosure.

FIG. 13 shows a graph and Western Blot illustrating functionality according to certain embodiments of the present disclosure.

FIG. 14 shows two graphs illustrating functionality of a specific embodiment according to one aspect of the present disclosure.

FIG. 15 shows two graphs illustrating functionality of a specific embodiment of the disclosure and according to an alternative aspect to FIG. 14.

FIG. 16 shows two graphs illustrating in vivo activity of certain aspects of the disclosure.

FIG. 17 shows two Western Blot images illustrating in vivo anti-tumour activity of certain aspects of the disclosure.

FIG. 18 shows a series of confocal microscopy images illustrating in vivo activity in the brain of certain aspects of the disclosure.

FIG. 19A shows a graphical representation of two VSV-G mutants.

FIG. 19B shows a set of Confocal microscopy images according to certain embodiments of the present disclosure.

FIG. 19C shows a Western Blot, illustrating functionality according to certain embodiments of the present disclosure.

FIG. 19D shows a series of graphs illustrating certain embodiments of the present disclosure.

FIG. 20A shows a series of graphs illustrating certain embodiments of the present disclosure.

FIG. 20B shows set of Confocal microscopy images according to certain embodiments of the present disclosure.

FIG. 20C shows a Western Blot, illustrating functionality according to certain embodiments of the present disclosure.

FIG. 21A shows a graph demonstrating functionality according to certain embodiments of the present disclosure.

FIG. 21B shows set of Confocal microscopy images according to certain embodiments of the present disclosure.

FIG. 21C shows a Western Blot, illustrating functionality according to certain embodiments of the present disclosure.

FIG. 22A shows a series of graphs illustrating certain embodiments of the present disclosure.

FIG. 22B shows set of Confocal microscopy images according to certain embodiments of the present disclosure.

FIG. 23A shows a graphical representation of constructs and reporter cells according to embodiments of the present disclosure.

FIG. 23B shows a series of graphs illustrating certain embodiments of the present disclosure.

FIG. 23C shows set of Confocal microscopy images according to certain embodiments of the present disclosure.

FIG. 23D shows a Western Blot, illustrating functionality according to certain embodiments of the present disclosure.

FIG. 24A shows set of Confocal microscopy images according to certain embodiments of the present disclosure.

FIG. 24B shows a graph illustrating certain embodiments of the present disclosure.

FIG. 24C shows a Western Blot, illustrating functionality according to certain embodiments of the present disclosure.

FIG. 25A shows a pictorial representation of a in vivo methods according to certain embodiments of the disclosure.

FIG. 25B shows set of Confocal microscopy images according to certain embodiments of the present disclosure.

FIG. 26 shows set of Confocal microscopy images according to certain embodiments of the present disclosure.

FIG. 27 shows set of Confocal microscopy images according to certain embodiments of the present disclosure.

FIG. 28A shows a 3D structure of super suppressor inhibitor of NF-κB according to the present invention.

FIG. 28B shows a series of graphs illustrating certain embodiments of the present disclosure.

FIG. 28C shows a Western Blot, illustrating functionality according to certain embodiments of the present disclosure.

FIG. 29A shows a graphical representation of the constructs and reporter cells used according to certain embodiments of the disclosure.

FIG. 29B shows a series of graphs illustrating certain embodiments of the present disclosure.

FIG. 29C shows a pictorial representation of a in vivo methods according to certain embodiments of the disclosure.

FIG. 29D shows a series of graphs illustrating certain embodiments of the present disclosure.

FIG. 29E shows a set of histology images illustrating certain embodiments of the present disclosure.

### DETAILED DESCRIPTION OF PREFERRED EMBODIMENTS

The present disclosure relates to EVs capable of improved delivery of cargo, wherein the EV comprises an endosomal escape enhancer. The present disclosure also relates to methods of making and purifying said EVs and their use in therapy.

The disclosure will be more clearly understood from the following description of some embodiments thereof, given by way of example only, and with reference to the Figures where appropriate.

All publications, patent applications, patents, and other references mentioned herein are incorporated by reference in their entirety. The references cited herein are not admitted as prior art to the claimed disclosure. In the case of conflict, the present specification, including definitions, will control.

In a first aspect of the present disclosure there is provided an engineered EV for delivering a bioactive cargo. The EV comprises an endosomal escape enhancer and a cargo.

Exemplary endosomal escape enhancers are discussed in more detail hereinbelow. The endosomal escape enhancer may be selected from VSV-G, CVG, PFV Envelope and heVLPs.

In one aspect, an endosomal escape enhancer as described herein mediates fusion between the engineered EV and endosomal membranes. Suitably, an endosomal escape enhancer as described herein exhibits fusogenic activity. In one aspect, an endosomal escape enhancer as described herein binds to low-density lipoprotein receptors (LDL-R) on the surface of a cell as described herein. In a preferred aspect, an endosomal escape enhancer as described herein exhibits fusogenic activity and binds to LDL-R on the surface of a cell as described herein.

An endosomal escape enhancer as described herein can comprise or consist of an amino acid sequence of any one of SEQ ID NOs: 13, 14 and 15 or a sequence having at least 80%, at least 85%, at least 90%, at least 95%, at least 96%, at least 97%, at least 98%, or at least 99% identity to any one of SEQ ID NOs: 13, 14 and 15, wherein the polypeptide exhibits fusogenic activity as described herein and/or binds to LDL-R on the surface of a cell as described herein, or a functional fragment or variant thereof. In a preferred aspect, an endosomal escape enhancer as described herein can comprise or consist of an amino acid sequence of SEQ ID NO: 13 or a sequence having at least 80%, at least 85%, at least 90%, at least 95%, at least 96%, at least 97%, at least 98%, or at least 99% identity to SEQ ID NO: 13, wherein the polypeptide exhibits fusogenic activity as described herein and/or binds to LDL-R on the surface of a cell as described herein, or a functional fragment or variant thereof.

An endosomal escape enhancer as described herein can be encoded by a polynucleotide sequence comprising or consisting of any one of SEQ ID NOs: 1, 2 and 3 or a sequence having at least 80%, at least 85%, at least 90%, at least 95%, at least 96%, at least 97%, at least 98%, or at least 99% identity to any one of SEQ ID NOS: 1, 2 and 3, wherein the sequence encodes a polypeptide exhibiting fusogenic activity and/or that binds to LDL-R on the surface of a cell as described herein, or a functional fragment or variant thereof. In various aspects, the polynucleotide constructs described herein, for example according to the second aspect, can comprise or consist of a polynucleotide sequence comprising or consisting of any one of SEQ ID NOS: 1, 2 and 3 or a sequence having at least 80%, at least 85%, at least 90%, at least 95%, at least 96%, at least 97%, at least 98%, or at least 99% identity to any one of SEQ ID NOs: 1, 2 and 3, wherein the sequence encodes a polypeptide exhibiting fusogenic activity and/or that binds to LDL-R on the surface of a cell as described herein, or a functional fragment or variant thereof. In a preferred aspect, an endosomal escape enhancer as described herein can be encoded by a polynucleotide sequence comprising or consisting of SEQ ID NO: 1 or a sequence having at least 80%, at least 85%, at least 90%, at least 95%, at least 96%, at least 97%, at least 98%, or at least 99% identity to SEQ ID NO: 1, wherein the sequence encodes a polypeptide exhibiting fusogenic activity and/or that binds to LDL-R on the surface of a cell as described herein, or a functional fragment or variant thereof.

Cargo in accordance with the present disclosure may include, but are not limited to, the following:

Essentially any type of therapeutic cargo, such as for instance: a nucleic acid such as an RNA molecule, a DNA molecule or a mixmer, mRNA, antisense or splice-switching oligonucleotides, siRNA, shRNA, saRNA, miRNA, plasmid DNA (pDNA), supercoiled or unsupercoiled plasmids, mini-circles, doggy-bond DNA (bdDNA), peptides or proteins including: transporters, enzymes, receptors such as decoy receptors, membrane proteins, cytokines, antigens and neoantigens, ribonuclear proteins, nucleic acid binding proteins (NA-binding proteins), antibodies, nanobodies, antibody fragments, antibody-drug conjugates, small molecule drugs, gene editing technology such as CRISPR-Cas9 or any other CRISPR enzymes, TALENs, meganucleases, or vesicle-based cargos such as viruses (e.g. AAVs, lentiviruses, naked viral genomes etc.). In one embodiment, the cargo may be a mixture of protein, nucleic acid, virus, viral genome, antigen and/or small molecule. In a preferred aspect, the cargo is a protein, preferably a Cas9 ribonucleoprotein (RNP), a meganuclease targeting PCSK9, a Super suppressor of NFκB or a recombinase enzyme, preferably Cre.

The engineered EV may also comprise a release system. The release system may be any system capable of physically linking components in a manner whereby the components may subsequently be separated and may be positioned for the release of the cargo. For instance, the release system may be a cleavable amino acid sequence within a protein construct or a fusion protein. In particular embodiments, the release system is part of a protein construct and is positioned between a cargo and an EV protein. In other embodiments, the release system is part of a protein construct and is positioned between either an endosomal escape enhancer or multimerization domain and a cargo. The release system may be positioned adjacent to the cargo, such that the cargo is released as an isolated component. For instance, the cargo may be released in a form that does not comprise amino acid sequences not forming part of the cargo, i.e., so that the cargo is released in free unconjugated form and thus optimally active. Alternatively, the cargo may be released in a substantially free form, wherein a portion of the release system may remain attached to the cargo but does not interfere with the desired functionality and/or activity of the cargo when released.

Release systems in accordance with the disclosure may include, but are not limited to, the following:

Cis-cleaving sequences (or self-cleaving) such as inteins, and mini-inteins, light induced monomeric or dimeric release domains such as Kaede, KikGR, EosFP, tdEosFP, mEos2, PSmOrange, the GFP-like Dendra proteins Dendra and Dendra2, CRY2-CIBN, etc. Alternatively, nuclear localization signal (NLS) -nuclear localization signal-binding protein (NLSBP) (NLS-NLSBP) release system may be employed. Protease cleavage sites may also be incorporated into the protein constructs and/or fusion proteins for spontaneous release, etc., depending on the desired functionality of the fusion polypeptide. It is to be appreciated that in certain embodiments the protein construct may be a fusion protein. In the case of nucleic acid cargos specific nucleic acid cleaving domains may be included. Non-limiting examples of nucleic acid cleaving domains include endonucleases such as Cas6, Cas13, engineered PUF nucleases, site specific RNA nucleases etc.

In particular examples, the release system may be a cis-cleaving sequence. The cis-cleaving sequence may be an amino acid sequence positioned between at least two domains of a protein construct or a fusion protein. For instance, the cis-cleaving sequence may be positioned between a cargo and an endosomal escape enhancer, a multimerization domain or an EV protein. A portion of the cis-cleaving sequence may also form part of the protein to which it is attached (i.e., a cargo, and an endosomal escape enhancer, a multimerization domain or an EV protein).

The cis-cleaving sequence may be a self-cleaving protein, for example an intein. The intein may be a slow-cleaving or a fast-cleaving intein. The intein may be a mini-intein, such as a mini-intein that has been modified to optimise the cleavage rate. The intein may be a delta-intein-CM. Thus, cis-cleaving sequences (or self-cleaving) in accordance with the disclosure may include, but are not limited to, the following:

Inteins, mini-inteins, delta inteins and certain variants, mutations and domains thereof having a desired functionality (including, but not limited to self-cleaving instead of splicing), such as a mini-intein modified to optimise the cleavage rate. For example, a mini-intein having C1A, D24G, V67L and/or D150G substitution in the N-terminal portion (or an appropriate—C-extein position). Said suitable substitutions can be found in SEQ ID No. 20. For example, splicing is enabled by the +1 position of the intein, wherein the +1 is Cys. Substitution of Cys with Ala in ΔI-CM removes the splicing capability and supports cleavage only. It will be appreciated that other mutations, substitutions, and the like may also similarly work. As such the substitution hereinbefore mentioned, while exemplary, is not limiting in any way and may also differ from intein to intein.

In certain instances, one may opt to utilize slow-cleaving inteins. A slow-cleaving system may be preferable when more time is required to ensure efficient loading of an EV with the desired cargo. Slow-cleaving inteins in accordance with the disclosure may include, but are not limited to, the following: mini-inteins, delta inteins, delta-intein-CM and a mini-intein and certain variants, mutations and domains thereof having a desired functionality (such as, but not limited to cleavage action instead of splicing and cleavage rate). In a preferred embodiment, the slow-cleaving cis-cleaving release system is based on an intein system, wherein the C-terminal portion of the intein may comprise the amino acid sequences Val-Val-Val-His-Asn, more preferably wherein the C-terminal portion of the intein is modified to comprise Val-Val-Val-His-Asn-Gly. Certain modifications at the +1 C-extein position, have been observed to slow the cleavage rate (i.e., are slow-cleaving).

In certain instances, one may opt to utilize a fast-cleaving cis-cleaving release system (such as a fast-cleaving cis-cleaving intein). A fast-cleaving system may be preferable when EVs need to be harvested quickly. In a preferred embodiment, the fast-cleaving cis-cleaving release system is based on an intein system, wherein the C-terminal portion of the intein may comprise the amino acid sequences Val-Val-Val-His-Asn or Val-Val-Val-His-Asn-Cys. Certain modifications at the +1 C-extein position, such as the abovementioned example, have been observed to speed up the cleavage rate (i.e., are fast-cleaving).

Advantages to inclusion of a release domain include, but are not limited to the following:

Release from parts or domains of the original fusion polypeptide is enabled.

This is particularly advantageous when the release from parts of the fusion polypeptide would increase bioactive delivery of the cargo and/or when a particular function of the fusion polypeptide works better when part of a smaller construct.

Another advantage, particularly when using self-cleaving proteins such as intein, is that a cleaner release of the cargo is made. It is to be appreciated that a self-cleaving intein will leave minimal, if any, amino acids attached to the cargo. This capability results in the cargo being released in a substantially free and unconjugated form meaning that the cargo is in an active/functional form.

This is particularly advantageous when using an intein, preferably a mini-intein having a mutation about the splicing domain(s), for example+C-extein V to L substitution or any other intein which has been modified (by mutation/substitution) such that the splicing capability is knocked out. It will be appreciated that a mini-intein modified in this way leaves the intein with the self-cleaving action.

Another advantage to using a self-cleaving system such as a suitably modified intein is that the cargo is released in an unconjugated form that is substantially free from amino acids attached to the cargo. The unconjugated cargo once released is thus less likely to induce an immune response and/or become immunogenic.

In one preferred embodiment the engineered EV also comprises an EV protein that may be an exosomal protein when the EV is an exosome.

Such EV protein (or exosomal protein) comprised in the engineered EVs as per the present disclosure may be selected from a wide variety of proteins, such as EV transmembrane proteins, EV membrane associated proteins, ICAMs, integrins, sydecans, syntenins, interleukin receptors, immunoglobulins, MHC-I or MHC-II components, GPI anchor proteins, ATP transporters, members of the myristoylated alanine rich protein kinase C substrate (MARCKS) protein family, matrix metalloproteinases (MMPs), TNFRs known at the time of the disclosure to be associated with EVs (or exosomes), as well as any combinations, derivatives, domains, variants, mutants, or regions thereof.

Transmembrane EV proteins may include, but are not limited to, the following non-limiting examples: CD9, CD53, CD63, CD81, CD82, CD54 (ICAM1), CD50 (ICAM3), CD49d (ITGA4), CD71, CD133, ALIX, Syntenin-1, Syntenin-2, Lamp2, Lamp2a, Lamp2b, TSN1, TSN3, TSN4, TSN5 TSN6, TSPAN8, TSN31, TSN10, TSN11, TSN12, TSN13, TSN14, TSN15, TSN16, TSN17, TSN18, TSN19, TSN2, TSN9, TSN32, TSN33, CD37, CD151, CD231 (TSN7; TSPAN7; TALLA-1, TM4SF2), NOTCH1, NOTCH2, NOTCH3, NOTCH4, Delta-Like Protein (Delta 1; DLL1), CD3 epsilon, CD34, CD40, CD47, CD86, CD115 (CSF1R), CD125, CD200, CD362 (syndecan 2), EGFR, GLUR2, GLUR3 (GRIA3), L1CAM, Syntaxin 3 (STX3), TFR1, UPK1A, UPK1B, VTI1A, VTI1B, CD184 (CXCR4), CD102, Integrin beta-5 (ITGB5), Integrin beta-6 (ITGB6), Integrin beta-7 (ITGB7), CD104, CD19, CD11a, CD11b, CD11c, CD235a, CD3zeta, CD41, CD49b (ITGA2), CD49c, CD49e, CD50, CD61, JAG2, CD18 (ITGB2), CD13, CD45, CD110, CD117, CD135, CD273, CD274, AGRN, HLA-DM, LFA-1, Mac-1alpha, CD36, CD279, TfR1, Syndecan 3, Syndecan 4, CD224, CLIC1, CLIC4, CD44, Prostaglandin F2 Receptor Negative Regulator (PTGFRN) (FPRP), GP130, HLAA, Limp2, MYOF, ATP2B2, ATP2B3, ATP2B4, IGSF2, IGSF3, IGSF8, ITGB1, ATP1A1, ATP1A2, ATP1A3, ATP1A4, ATP1B3 (AT1B3), SLC3A2, MMP14, PDGFR, PRPH2, ROM1, CD55, or any single-pass or multi-pass transmembrane protein and/or tetraspanin, or any other EV protein and/or exosomal protein known at the time of the disclosure that spans the EV membrane (or exosomal membrane).

Membrane associated EV proteins in accordance with the disclosure may include, but are not limited to, the following non-limiting examples: CD2, CD138 (Syndecan 1), CD40L, Delta-Like Protein (Delta 4; DLL4), Jaggard 1 (JAG1), MFGE8 (lactoadherin; LA), FLOT1, FLOT2, SLIT2, TCRA, CD117 (isoform 3), GAPDH, AT2B4, BASP1, BSG, ARRDC1, TCRA, TCRB, TCRD, TCRG, or any other EV protein and/or exosomal protein known at the time of the disclosure that is associated with the EV/exosomal membrane, plasma membrane or lysosomal membrane from which the EV/exosome has been created.

Other EV proteins in accordance with the disclosure may include, but are not limited to, the following non-limiting examples: LGALS3BP, COL6A1, LAMB1, LAMC1, HSPG2, ANNEXIN, CD111, AAAT, BAS1, CD45RA, GTR1, ITA3, ITGA3, Mac-1beta, MARCKSL1, SSEA4, TCRB, TORD, TCRG, or any other EV protein and/or exosomal protein known at the time of the disclosure that is particularly enriched in EVs and/or exosomes.

Where the EV protein (or exosomal protein) is a transmembrane protein, it may be either a single-pass transmembrane protein or a multi-pass transmembrane protein.

Single-pass transmembrane EV proteins in accordance with the disclosure may include, but are not limited to, the following non-limiting examples: AGRN, ALIX, CD11a, CD11b, CD11c, CD13, CD18 (ITGB2), CD19, CD102, CD104, CD110, CD115 (CSF1R), CD117, CD125, CD135, CD200, CD235a, CD273, CD274, CD3 epsilon, CD3zeta, CD34, CD362 (Syndecan 2), CD40, CD41, CD44, CD45, CD49b (ITGA2), CD49c, CD49d (ITGA4), CD49e, CD50 (ICAM3), CD51, CD54 (ICAM1), CD61, CD71, CD86, Delta-Like Protein 1 (Delta 1; DLL1), EGFR, Integrin beta-5 (ITGB5), Integrin beta-6 (ITGB6), Integrin beta-7 (ITGB7), L1CAM, Lamp2, Lamp2a Lamp2b, NOTCH1, NOTCH2, NOTCH3, NOTCH4, Syntaxin (STX3), Syntenin 1, Syntenin 2, CD30, JAG2, HLA-DM, LFA-1, MAC-1alpha, VTI1A, VTI1B, AT1B3, BSG, TfR1, Syndecan 3, Syndecan 4, CD224, CLIC1, CLIC4, PTGFRN (FPRP), GP130, HLAA, MYOF, IGSF2, IGSF3, IGSF8, ITGB1, AT1B3 (ATP1B3), SLC3A2, MMP14, PDGFR, CD55, or any other EV protein and/or exosomal protein known at the time of the disclosure that spans the EV membrane (or exosomal membrane) only once.

Multi-pass transmembrane EV proteins in accordance with the present disclosure may include, but are not limited to, the following non-limiting examples: GLUR2, GLUR3 (GRIA3), CD47, CD133, CD151, CD184 (CXCR4), CD231 (TSPAN7; TALLA-1; TM4SF2); CD37, CD53, CD63, CD81, CD82, CD9, TSN1, TSN3, TSN4, TSN5 TSN6, TSPAN8, TSN31, TSN10, TSN11, TSN12, TSN13, TSN14 (TSPAN14), TSN15, TSN16, TSN17, TSN18, TSN19, TSN2, TSN9, TSN32, TSN33, CD36, CD279, AT2B4, Limp2, ATP2B1, ATP2B2, ATP2B3, ATP2B4, ATP1A1, ATP1A2, ATP1A3, ATP1A4, PRPH2, ROM1, UPK1A, UPK1B, or any other EV protein and/or exosomal protein known at the time of the disclosure that spans the EV membrane (or exosomal membrane) more than once.

When a multi-pass transmembrane protein is the EV protein (or exosomal protein) it may particularly include a tetraspanin. Tetraspanins in accordance with the present disclosure may include, but are not limited to, the following non-limiting examples: CD231 (TSPAN7; TALLA-1; TM4SF2); CD37, CD53, CD63, CD81, CD82, CD9, TSN1, TSN3, TSN4, TSN5 TSN6, TSPAN8, TSN31, TSN10, TSN11, TSN12, TSN13, TSN14 (TSPAN14), TSN15, TSN16, TSN17, TSN18, TSN19, TSN2, TSN9, TSN32, TSN33, ROM1 (TSN23), UPK1A (TSN21), UPK1B (TSPAN20), or any other EV protein and/or exosomal protein known at the time of the present disclosure that spans the EV membrane (or exosomal membrane) four times (i.e., 4× membrane domains) and has two extravesicular loops displayed on the surface (or outer leaflet) of the EV membrane (or exosomal membrane) where one of the loops is a larger outward-facing loop and the second loop of the two loops is a smaller outward-facing loop.

Particularly advantageous EV proteins include the tetraspanins, CD63, CD81, CD9, CD82, and the single-pass transmembrane EV protein PTGFRN.

Mutations may be introduced into the wild-type sequence of the EV protein to alter its function. A preferred mutant according to the disclosure is CD63 (Y235A).

Advantages of an EV protein (or exosomal protein) includes, but are not limited to, the following:
Including an EV protein (e.g., exosomal protein) in the protein construct helps to actively load the cargo into the EV (or exosome).
The EV protein (e.g., exosomal protein) traffics a protein construct, which may comprise other protein domains with desired utility (e.g., a release system, preferably a self-cleaving intein), such as part of a fusion protein to an EV membrane.

The use of EV proteins has the effect of driving loading of the protein construct into EVs such that protein construct is actively loaded into EVs; as a result, EV proteins are sometimes referred to as carrier proteins.

In a particular embodiment, there is provided an engineered EV suitable for delivery of at least one bioactive cargo. The EV comprises a protein construct or a fusion protein which comprises a cargo, an EV protein, and a release system, wherein the release system is positioned in-between the cargo and the EV protein. The release system may be adjacent to the cargo, such that the cargo is released as either an isolated component or substantially free form. In a particular embodiment, the release system is a cis-cleaving amino acid sequence. The EV further comprises an endosomal escape enhancer, for instance VSV-G. The EV may be an exosome and the EV protein may be an exosomal protein.

In a preferred embodiment the domains of the protein construct or fusion protein are arranged from N terminal (NTD) to C-terminal (CTD) as per the following non-limiting examples:
NTD-EV protein-cargo-CTD
NTD-EV protein-release system-cargo-CTD
NTD-VSVG-release system-cargo-CTD

It will be understood that, in this embodiment, where an EV comprises a construct comprising an EV protein, a cargo and an optional release system (optionally intein), the EV also comprises a separate construct comprising an endosomal escape enhancer.

In one embodiment the endosomal escape enhancer (e.g., VSV-G) may be displayed on the surface of the EV and the cargo may be loaded into the lumen of the EV. It will be appreciated that this may or may not be achieved by a single construct or double construct depending on NTD, CTD and/or intraloop positioning of the cargo/endosomal escape enhancer and without further modification, such as the addition of a release system. This arrangement (i.e., endosomal escape enhancer displayed on the surface and cargo loaded into the lumen of the EV) has the advantage that steric hindrance between the endosomal escape enhancer and the cargo is avoided, which the inventors postulate contribute to the stability of, for example, multiple protein constructs wherein at least one protein construct comprises a cargo and the other construct comprises an endosomal escape enhancer (i.e., a double-stable protein construct) and to enhanced endosomal escape and delivery of a functional/active cargo to the desired site of action (e.g., the cytosol of a cell) by either a double-stable or single construct.

In an alternative embodiment the engineered EV, instead of having an EV protein, may comprise a construct comprising both the endosomal escape enhancer and the cargo. It will be appreciated that an endosomal escape enhancer may comprise a membrane domain that is capable of trafficking the remaining endosomal escape enhancer to the EV membrane (or exosomal membrane). In this instance a separate EV protein may not be required.

In such embodiments, the construct may also comprise a multimerization domain, such as a trimerization domain like foldon. A multimerization domain is a domain that enables dimerization, trimerization, or any higher order of multimerization of protein constructs containing said domain. The multimerization domain brings additional stability to the protein construct.

Multimerization domains in accordance with the disclosure may include, but are not limited to, the following:
leucine zipper, foldon domain, fragment X, collagen domain, 2G12 lgG homodimer, mitochondrial antiviral-signaling protein caspase activation and recruitment domain (CARD) filament, Cardiac phospholamban transmembrane pentamer, parathyroid hormone dimerization domain, Glycophorin A transmembrane, human immunodeficiency virus (HIV) Gp41 trimerisation domain, human papillomavirus 45 (HPV45) oncoprotein E7 C-terminal dimer domain, and any combination thereof.

One advantage of a multimerization domain is that it enables dimerization, trimerization, or any higher order of multimerization of the fusion polypeptides, which increases the sorting and trafficking of the fusion polypeptides into EVs and may also contribute to increase the yield of vesicles produced by EV-producing cells.

The engineered EV of the alternative embodiment, comprising a construct comprising an endosomal escape enhancer and a cargo, may also comprise a release system, as disclosed herein. The release system may be positioned for the release of the cargo.

The inventors have found that a fusion protein formed comprising an endosomal escape enhancer, a cargo, and optionally a release system (such as intein) is surprisingly more effective if a multimerization domain is employed.

In this alternative embodiment the endosomal escape enhancer (such as VSV-G) acts in a similar way to the EV protein (or exosomal protein) with much the same advantages as herein discussed.

Advantages include, but are not limited to the following:
Issues associated with two or more protein constructs of protein misfolding, toxic protein aggregation, translational errors, imperfect association/dissociation between the proteins is avoided.
VSV-G both traffics cargo to the EV membrane, optionally loading it into the lumen of the EV, and provides endosomal escape functionality.
The protein construct is smaller in size meaning greater stability of the construct in the EV membrane and lower chances of the protein construct from dissociation from the membrane.

Where the cargo is lumen side and/or associated with the inner leaflet of the EV membrane even greater stability is imparted since the cargo and the endosomal escape enhancer are not in steric competition with each other.

It will be appreciated that a multimerization domain is not essential for formation of this single protein construct and thus the associated advantages.

Only one plasmid is needed reducing any stability issues in cell culture and reducing any subsequent issues with the production of EVs (or exosomes).

Only one protein construct, preferably as a fusion protein, is loaded into the EV (or exosome) meaning that the engineered EV design is simplified, and limits disruption of the EV (or exosomal) membrane and other components contained therein.

The combination of the VSV-G and a multimerization domain enable the formation of a more stable single protein construct further comprising the cargo of interest enabling improved functionality. In embodiments where a release system is included, such as a self-cleaving protein (e.g., intein). The addition of the release system adds further benefits as herein described.

This alternative embodiment is particularly advantageous over known approaches which either lack the ability to escape the endosomal pathway (which is desired for functional delivery) and/or require two constructs and two plasmids with all the downsides associated with that as herein described.

The inventors have surprisingly found that several endosomal escape enhancers work equally well lending to an advantage of a modular approach in the production of EVs which can be adapted to the desired need of the product (including, but not limited to, cell specific targeting).

One advantage of heVLPs is that immunogenicity is reduced.

One or more of either the endosomal escape enhancer or the cargo may independently form part of a fusion protein with an EV protein. Optionally the EV is an exosome, and the EV protein is an exosomal protein.

Optionally the EV may comprise further protein constructs or fusion proteins where a second EV protein, or exosomal protein may be selected from an EV transmembrane protein or EV membrane associated protein, ICAMS, integrins, syndecans, syntenins, interleukin receptors, immunoglobulins, MHC-I or MHC-II components, GPI anchor proteins, ATP transporters, members of the myristoylated alanine-rich protein kinase C substrate (MARCKS) protein family, matrix metalloproteinases (MMPs), TNFRs known at the time of the disclosure to be associated with EVs (or exosomes).

Transmembrane EV proteins in accordance with the disclosure may include, but are not limited to, the following non-limiting examples: ALIX, CD115 (CSF1R), CD125, CD200, CD3 epsilon, CD34, CD362 (Syndecan 2), CD40, CD49d, CD50 (ICAM3), CD54 (ICAM1), CD71, CD86, Delta-Like Protein (Delta 1; DLL1), EGFR, L1CAM, Lamp, Lamp2a, Lamp2b, NOTCH 1, NOTCH 2, NOTCH 3, NOTCH 4, Syntaxin 3 (STX3), Syntenin 1, Syntenin 2, GLUR2, GLUR3 (GRIA3), CD47, CD133, CD151, CD184 (CXCR4), CD231 (TSPAN7; TALLA-1; TM4SF2), CD37, CD53, CD63, CD81, CD82, CD9, TSN1, TSN3, TSN4, TSN5, TSN6, TSAPN8, TSN31, TSN10, TSN11, TSN12, TSN13, TSPAN14, TSN15, TSN16, TSN17, TSN18, TSN19, TSN2, TSN9, TSN32, TSN33, TfR1, UPK1A, UPK1B, VTI1A, VTI1B, CD102, integrin beta-5 (ITGB5), integrin beta-6 (ITGB6), integrin beta-7 (ITGB7), CD104, CD19, CD11a, CD11b, CD11c, CD235a, CD3zeta, CD41, CD49b (ITGA2), CD49c, CD49e, CD61, JAG2, CD18, (ITGB2), CD13, CD45, CD110, CD117, CD135, CD273, CD274, AGRN HLA-DM, LFA-1, Mac-1alpha, CD36, CD279, TfR1, syndecan 3, syndecan 4, CD224, CLIC1, CLIC4, CD44, PTGFRN (FPRP), GP130, HLAA, Limp2, MYOF, ATP2B2, ATP2B3, ATP2B4, IGSF2, IGSF3, IGSF8, ITGB1, ATP1A1, ATP1A2, ATP1A3, ATP1A4, ATP1B3 (AT1B3), SLCO3A2, MMP14, PDGFR, PRPH2, ROM1, CD55, or any single-pass or multi-pass transmembrane protein and/or tetraspanin or any other EV protein and/or exosomal protein known at the time of disclosure that spans the EV membrane (or exosomal membrane).

Membrane-associated proteins in accordance with the disclosure may include, but are not limited to, the following non-limiting examples: CD2, CD138 (Syndecan 1), CD40L, Delta-Like Protein (Delta 4; DLL4), Jaggard 1 (JAG1), MFGE8 (lactoadherin; LA), FLOT1, FLOT2, SLIT2, TCRA, CD117 (isoform 3), GAPDH, AT2B4, BASP1, BSG, ARRDC1, TCRA, TCRB, TCRD, TCRG or any other EV protein and/or exosomal protein known at the time of disclosure that is associated with the EV/exosomal membrane, plasma or lysomosal membrane from which the EV/exosome has been created.

Where the EV protein (or exosomal protein) is a transmembrane protein, it may be either a single-pass transmembrane protein or a multi-pass transmembrane protein.

Single-pass transmembrane proteins in accordance with the disclosure may include, but are not limited to, the following non-limiting examples: AGRN, ALIX, CD11a, CD11b, CD11c, CD13, CD18, (ITGB2), CD19, CD102, CD104, CD110 CD115 (CSF1R), CD117, CD125, CD135, CD200, CD235a, CD273, CD274, CD3 epsilon, CD3zeta, CD34, CD362 (Syndecan 2), CD40, CD41, CD44, CD45, CD49b (ITGA2), CD49c, CD49d, CD49e CD50 (ICAM3), CD51, CD54 (ICAM1), CD61, CD71, CD86, Delta-Like Protein (Delta 1; DLL1), EGFR, integrin beta-5 (ITGB5), integrin beta-6 (ITG6), integrin beta 7 (ITGB7), L1CAM, Lamp2, Lamp2a, Lamp2b, NOTCH 1, NOTCH 2, NOTCH 3, NOTCH 4, Syntaxin 3 (STX3), Syntenin 1, Syntenin 2, CG30, JAG2, HLA-DM, LFA-1, MAC-1alpha, VTI1A, VTI1B, AT1B3, BSG, TfR1, syndecan 3, syndecan 4, CD224, CLIC1, CLIC4, PTGFRN (FPRP), GP130, HLAA, MYOF, IGSF2, IGSF3, IGSF8, ITB1, SLC3A2, MMP14, PDGFR, CD55, or any other EV protein and/or exosomal protein known at the time of disclosure that spans the EV membrane (or exosomal membrane) only once.

Multi-pass transmembrane proteins in accordance with the disclosure may include, but are not limited to, the following non-limiting examples: GLUR2, GLUR3 (GRIA3), CD47, CD133, CD151, CD184 (CXCR4), CD231 (TSPAN7; TALLA-1; TM4SF2), CD37, CD53, CD63, CD81, CD82, CD9, TSN1, TSN3, TSN4, TSN5, TSN6, TSAPN8, TSN31, TSN10, TSN11, TSN12, TSN13, TSPAN14, TSN15, TSN16, TSN17, TSN18, TSN19, TSN2, TSN9, TN32, TSN33, CD36, CD279, AT2B4, Limp2, ATP2B1, ATP2B2, ATP2B3, ATP2B4, ATP1A1, ATP1A2, ATP1A3, ATP1A4, PRPH2, ROM1, UPK1A, UPK1B, or any other EV protein and/or exosomal protein known at the time of disclosure that spans the EV membrane (or exosomal membrane) more than once.

When a multi-pass transmembrane protein is the EV protein (or exosomal protein) it may include a tetraspanin. Tetraspanins in accordance with the disclosure may include, but are not limited to, the following non-limiting examples: CD231 (TSPAN7; TALLA-1; TM4SF2), CD37, CD53, CD63, CD81, CD82, CD9, TSN1, TSN3, TSN4, TSN5, TSN6, TSAPN8, TSN31, TSN10, TSN11, TSN12, TSN13, TSPAN14, TSN15, TSN16, TSN17, TSN18, TSN19, TSN2, TSN9, TN32, TSN33, CD36, CD279, AT2B4, Limp2, ATP2B1, ATP2B2, ATP2B3, ATP2B4, ATP1A1, ATP1A2, ATP1A3, ATP1A4, PRPH2, ROM1 (TSN23), UPK1A (TSN21), UPK1B (TSPAN20), or any other EV protein and/or exosomal protein known at the time of disclosure that spans the EV membrane (or exosomal membrane) four times (i.e., 4×membrane domains) and has two extravesicular loops displayed on the surface (or outer leaflet) of the EV membrane (or exosomal membrane) where one of the two loops is a larger outward-facing loop and the second loop of the two loops is a smaller outward facing loop.

Preferred EV proteins may include the tetraspanins CD63, CD9, CD81 and the single-pass transmembrane Prostaglandin F2 Receptor Negative Regulator (PTGFRN).

Mutations may be introduced into the wild-type sequence of the EV protein to alter its function. A preferred mutant according to the disclosure is CD63 (Y235A).

One advantage of an EV protein (or exosomal protein) is that when attached (preferably fused) to another protein of interest (such as cargo, release system or endosomal escape enhancer) the protein of interest is readily trafficked to the EV membrane (or exosomal membrane) utilising the naturally existing infrastructure of an EV (or exosome).

The cargo may include proteins (e.g., Cre), CRISPR-associated proteins such as Cas9 and/or super suppressors of NFkB such as IKBalpha or any variants, modifications, mutations/substitutions and/or domains.

In a particular embodiment, there is provided an engineered EV suitable for delivery of at least one bioactive cargo. The EV comprises a protein construct or a fusion protein which comprises an endosomal escape enhancer, a multimerization domain, a cargo, and a release system, wherein the release system is positioned in-between the cargo and the endosomal escape enhancer. In some embodiments, the endosomal escape enhancer and the multimerization domain are positioned on one side of the release system, and the cargo is positioned on the other side. The release system may be adjacent to the cargo, such that the protein is released as either an isolated component or a substantially free form. In a particular embodiment, the endosomal escape enhancer is VSV-G, and the release system is a cis-cleaving amino acid sequence, most preferably ΔI-CM. The multimerization domain may be foldon. The EV may be an exosome and the EV protein may be an exosomal protein.

In a preferred embodiment the domains of the protein construct or fusion protein are arranged from N terminal (NTD) to C-terminal (CTD) as per the following non-limiting examples:
NTD-endosomal escape enhancer-cargo-CTD
NTD-endosomal escape enhancer-release system-cargo-CTD
NTD-endosomal escape enhancer-multimerization domain-cargo-CTD
NTD-endosomal escape enhancer-multimerization domain-release system-cargo-CTD

In one embodiment the VSVG may be displayed on the surface of the EV and the cargo may be loaded into the lumen of the EV. It will be appreciated that this may or may not be achieved by a single or double construct. This arrangement has the advantage that steric hindrance between the endosomal escape enhancer and the cargo is avoided, which the inventors postulate contribute to the stability of a double-stable construct and to enhanced endosomal escape and delivery of a functional/active cargo to the desired site of action (e.g., the cytosol of a cell) by either a double-stable or single construct.

Cargos in accordance with the disclosure may include, but are not limited to, the following non-limiting examples:

### Protein Cargos, Such as:

Antibodies, intrabodies, nanobodies, scFvs, affibodies, bi- and multispecific antibodies or binders including bispecific T-cell engagers (BiTEs), receptors, ligands, transporters, enzymes for e.g. enzyme replacement therapy (ERT) or gene editing, tumour suppressors (e.g., PTEN), viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins such as cytokines and inhibitors of such cytokines (cytokines may include: CXCL8, GMCSF, interleukins including: IL-1 family, IL-2, IL-4, IL-6, IL-6-like, IL-9, IL-10, IL12, IL-13, IL-17, Interferons including INF-alpha/beta/gamma, TNF family members, CD40 and CD40L, TRAIL, and TGF-beta family) mitochondrial proteins, and heat shock proteins, etc. The cargo may be a reporter protein such as GFP or nanoLuc. In one preferred embodiment, the encoded protein is a CRISPR-associated (Cas) polypeptide (such as Cas6, Cas9, Cas12, Cas12a/cpf1, Cas13) with intact nuclease activity which is associated with (i.e., carries with it) an RNA strand that enables the Cas polypeptide to carry out its nuclease activity in a target cell once delivered by the peptide. Alternatively, in another preferred embodiment, the Cas polypeptide may be catalytically inactive, to enable targeted genetic engineering. Yet another alternative may be any other type of CRISPR effector such as the single RNA guided endonuclease Cpf1. The inclusion of Cpf1 is a particularly preferred embodiment as herein disclosed, as it cleaves target DNA via a staggered double-stranded break. Cpf1 may be obtained from species such as Acidaminococcus or Lachnospiraceae. In yet another exemplary embodiment, the Cas polypeptide may also be fused to a transcriptional activator (such as the P3330 core protein), to specifically induce gene expression.

It will be appreciated that size may be a factor in choosing the most suitable protein cargos. In certain embodiments the cargo is no more than about 160 kDa, preferably no more than about 120 kDa, and most preferably no more than about 80 kDa. It will be appreciated that, in general, the smaller the protein the easier the delivery of the protein.

The cargo may be a protein cargo. The protein cargo may be a protein capable of binding to a second cargo, for instance a nucleic acid. Thus, the cargo linked to the EV protein or the endosomal escape enhancer, either directly or via a multimerization domain and/or intein, may be a nucleic acid (NA)-binding protein.

Loading nucleic acid cargos using nucleic acid binding proteins: The present disclosure also relates in some embodiments to nucleic acid cargos which are loaded by fusion of an EV protein to a NA-binding protein which then binds to the nucleic acid cargo molecule and causes loading of the nucleic acid cargo into the EV. Alternatively, where the EV protein is replaced with the endosomal escape enhancer (e.g., VSV-G) certain embodiments relate to nucleic acid cargos which are loaded by fusion of an endosomal escape enhancer to a NA-binding protein.

### NA-Binding Proteins Such as:

hnRNPA1, hnRNPA2B1, DDX4, ADAD1, DAZL, ELAVL4, IGF2BP3, SAMD4A, TDP43, FUS, FMR1, FXR1, FXR2, EIF4A13, the MS2 coat protein, as well as any domains, parts or derivates, thereof. More broadly, particular subclasses of RNA-binding proteins and domains, e.g., mRNA binding proteins (mRBPs), pre-rRNA-binding proteins, tRNA-binding proteins, small nuclear or nucleolar RNA-binding proteins, non-coding RNA-binding proteins, miRNA-binding proteins, shRNA-binding proteins and transcription factors (TFs). Furthermore, various domains and derivatives may also be used as the NA-binding domain to transport a nucleic acid cargo into EVs. Non-limiting examples of RNA-binding domains include small RNA-binding domains (RBDs) (which can be both single-stranded and double-stranded RBDs (ssRBDs and dsRBDs) such as DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1CTR, PAM2, Xpo1, Piwi, CSD, and Ribosomal_L7Ae. Such RNA-binding domains may be present in a plurality, alone or in combination with others, and may also form part of a larger RNA-binding protein construct as such, as long as their key function (i.e., the ability to transport a nucleic acid cargo of interest, e.g., an mRNA or a short RNA) is maintained.

In preferred embodiments the present disclosure relates to two groups of NA-binding domains, namely Pumilio and FBF (PUF) proteins and Cas polypeptides, specifically Cas6 and Cas13, as well as various types of NA-binding aptamers. PUF proteins are typically characterized by the presence of eight consecutive PUF repeats, each of approximately 40 amino acids, often flanked by two related sequences, Csp1 and Csp2. Each repeat has a ‘core consensus’ containing aromatic and basic residues. The entire cluster of PUF repeats is required for RNA binding. Remarkably, this same region also interacts with protein co-regulators, and is sufficient to rescue, to a large extent, the defects of a PUF protein mutant, which makes the PUF proteins highly suitable for mutations used in the present disclosure. Furthermore, PUF proteins are highly preferred examples of releasable NA-binding domains which bind with suitable affinity to nucleic acid cargo molecules, thereby enabling a releasable, reversible attachment of the PUF protein to the nucleic acid cargo. PUF proteins are found in most eukaryotes and are involved in embryogenesis and development. PUFs have one domain that binds RNA that is composed of 8 repeats generally containing 36 amino acids, which is the domain typically utilized for RNA binding in this patent application. Each repeat binds a specific nucleotide, and it is commonly the amino acid in position 12 and 16 that confers the specificity with a stacking interaction from amino acid 13. The naturally occurring PUFs can bind the nucleotides adenosine, uracil and guanosine, and engineered PUFs can also bind the nucleotide cytosine. Hence the system is modular and the 8-nucleotide sequence that the PUF domain binds to can be changed by switching the binding specificity of the repeat domains. Hence, the PUF proteins as per the present disclosure can be natural or engineered to bind anywhere in an RNA molecule, or alternatively one can choose PUF proteins with different binding affinities for different sequences and engineer the RNA molecule to contain said sequence. There is furthermore engineered and/or duplicated PUF domains that bind 16-nucleotides in a sequence-specific manner, which can also be utilized to increase the specificity for the nucleic acid cargo molecule further. Hence the PUF domain can be modified to bind any sequence, with different affinity and sequence length, which make the system highly modular and adaptable for any RNA cargo molecule as per the present disclosure. PUF proteins and regions and derivatives thereof that may be used as NA-binding domains as per the present disclosure include the following non-limiting list of PUF proteins: FBF, FBF/PUF-8/PUF-6,-7,-10, all from C. elegans; Pumilio from D. melanogaster; Puf5p/Mpt5p/Uth4p, Puf4p/Ygl014wp/Ygl023p, Puf5p/Mpt5p/Uth4p, Puf5p/Mpt5p/Uth4p, Puf3p, all from S. cerevisiae; PufA from Dictyostelium; human PUM1 (Pumilio 1, sometimes known also as PUF-8R) and any domains thereof, polypeptides comprising NA-binding domains from at least two PUM1, any truncated or modified or engineered PUF proteins, such as for instance PUF-6R, PUF-9R, PUF-10R, PUF-12R, and PUF-16R or derivatives thereof; and X-Puf1 from Xenopus. Particularly suitable NA-binding PUFs as per the present disclosure includes the following: PUF 531, PUF mRNA loc (sometimes termed PUFengineered or PUFeng), and/or PUFx2, (sequences of which are available in PCT/EP2018/080681) and any derivatives, domains, and/or regions thereof. The PUF/PUM proteins are highly advantageous as they may be selected to be of human origin. Furthermore, as is the case with the PUF proteins, Cas proteins such as Cas 6 and Cas13 are highly preferred examples of releasable NA-binding domains which bind with suitable affinity to nucleic acid cargo molecules, thereby enabling a releasable, reversible attachment of the Cas protein to the nucleic acid cargo. As with the PUF-based NA-binding domains, the Cas proteins represent a releasable, irreversible NA-binding domain with programmable, modifiable sequence specificity for the target nucleic acid cargo molecule, enabling higher specificity at a lower total affinity, thereby allowing for both loading of the nucleic acid cargo into EVs and release of the nucleic acid cargo in a target location.

Additional preferred embodiments include therapeutic protein cargos selected from the group comprising enzymes or transporters for lysosomal storage disorders.

### Enzymes or Transporters, Such as:

glucocerebrosidases such as imiglucerase, alpha-galactosidase, alpha-L-iduronidase, iduronate-2-sulfatase and idursulfase, arylsulfatase, galsulfase, acid-alpha glucosidase (GAA), sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alphaN-acetyl neuraminidase, GlcNAc phosphotransferase, mucolipin1, palmitoylprotein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase.

Additional preferred embodiments include therapeutic protein cargos selected from the group comprising enzymes associated with Urea cycle disorders including: N-Acetylglutamate synthase, carbamoyl phosphate synthetase, ornithine transcarbamoylase, argininosuccinic acid synthase, argininosuccinic acid lyase, arginase, mitochondrial ornithine transporter, citrin, y+L amino acid transporter 1, uridine monophosphate synthase UMPS.

In other preferred embodiments, the cargo may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as Dystrophin, mini-dystrophin, micro-dystrophin, utrophin, titin, nebulin, dystrophin-associated proteins such as dystrobrevin, syntrophin, syncoilin, desmin, sarcoglycan, dystroglycan, sarcospan, agrin, and/or fukutin. The cargos are typically proteins or peptides of human origin unless indicated otherwise by their name, any other nomenclature, or as known to a person skilled in the art, and they can be found in various publicly available databases such as Uniprot, RCSB, etc.

In another preferred embodiment the cargo is an antigen/neoantigen, optionally wherein the antigen/neoantigen is suitable for use in cancer immunotherapy.

Any antigen/neoantigen may be incorporated into the EVs of the present disclosure. The antigens may be suitable for raising immune responses against pathogens such as bacteria, viruses, funguses or the antigen may be a tumor antigen useful in eliciting an immune response against a tumor for cancer immunotherapy. There may be one or more antigens/neoantigens present in any EV according to the disclosure. The one or more antigens/neoantigens may be endogenous/autologous (coming from the subject itself) or exogenous/allogenic (coming from another subject) or in the case of more antigens/neoantigens being incorporated into/onto the EVs the antigens/neoantigens may be any mix of autologous/allogenic antigens. Preferably the antigens are autologous. Moreover, the one or more antigens/neoantigens may have any origin such as e.g., viral or bacterial or may be a tumour antigen and furthermore may be immunostimulatory or immunosuppressive or a combination thereof. The antigen/neoantigen may be be useful in the treatment of any disease by immunotherapy. The treatment of cancer by immunotherapy is a particularly preferred embodiment. Where the antigen is a neo-antigen, it may be identified by sequencing of a tumour to identify the neo-antigen.

Exemplary tumour antigens are: Alphafetoprotein (AFP), Carcinoembryonic antigen (CEA), CA-125, MUC-1, Epithelial tumor antigen (ETA), Melanoma-associated antigen (MAGE), WT-1, NY-ESO-1, LY6K, IMP3, DEPDC1, CDCA-1, abnormal products of ras, p53, KRAS, or NRAS, CTAG1B, peptides derived from chromosomal translocations such as BCR-ABL or ETV6-AML1, viral antigens such as peptides from HPV-related cancers, peptides derived from proteins such as tyrosinase, gp100/pmel17, Melan-A/MART-1, gp75/TRP1, or TRP2, and overexpressed antigens such as MOK (RAGE-1), ERBB2 (HER2/NEU).

Where the cargo is an antigen or neoantigen the EV or pharmaceutical composition comprising the EV may optionally further comprise at least one adjuvant. Where the antigen is administered with an adjuvant to stimulate the immune response the adjuvant may be: an inorganic compound: such as aluminium hydroxide, aluminium phosphate, calcium phosphate hydroxide, a mineral oil such as paraffin oil, bacterial products such as killed bacteria Bordetella pertussis, Mycobacterium bovis, toxoids, a nonbacterial organic such as squalene, a detergent such as Quil A, a plant saponin, a cytokine such as IL-1, IL-2, IL-12, or RIBI (muramyl dipeptides) or immunostimulating complexes (ISCOM) such as stimulator of interferon genes (STING) agonists which can include cyclic dinucleotides. Such adjuvants may protect the therapeutic EV from rapid dispersal by sequestering it in a local deposit, or they may contain substances that stimulate the host to secrete factors that are chemotactic for macrophages and other components of the immune system. Adjuvants that can be incorporated to a vaccine are well-known by the person skilled in the art and will be selected, in such a way that they do not negatively affect the immunological activity of the EV.

### Nucleic Acid (NA) Cargos Such as:

shRNA, siRNA, saRNA, miRNA, an anti-miRNA, mRNA, modified mRNA, gRNA, pri-miRNA, pre-miRNA, circular RNA, piRNA, tRNA, rRNA, snRNA, lncRNA, ribozymes, mini-circle DNA, plasmid DNA, RNA/DNA vectors, trans-splicing oligonucleotides, splice-switching oligonucleotides, CRISPR guide strands, morpholinos (PMO), antisense oligonucleotides (ASO), peptide-nucleic acids (PNA), a viral genome and viral genetic material (for instance a naked AAV genome), but essentially any type of nucleic acid molecule can be delivered by the EVs of the present disclosure. Both single-stranded and double-stranded nucleic acid molecules are within the scope of the present disclosure, and the nucleic acid molecule may be naturally occurring (such as RNA or DNA) or may be a chemically synthesised RNA and/or DNA molecule which may comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, thionucleotides, phosphoramidate, PNA, PMO, etc.

### RNA Cargos, Such as:

mRNA the mRNA may be a naturally or non-naturally occurring mRNA. An mRNA may include one or more modified nucleobases, nucleosides, or nucleotides. A nucleobase of an mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. Thus, a nucleobase may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine. A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase and/or sugar component. A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein. For example, “guanosine triphosphate” should be understood to include the canonical guanosine triphosphate, 7-methylguanosine triphosphate, or any other definition encompassed herein. An mRNA may include a 5′ untranslated region (UTR), a 3′ UTR, and/or a coding or translating sequence, which is translated to create the fusion protein of the present disclosure. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In some embodiments, an mRNA may include a 5′ cap structure, a chain terminating nucleotide, a stem loop, a polyA sequence, and/or a polyadenylation signal. A cap structure or cap species is a compound including two nucleoside moieties joined by a linker which caps the mRNA at its 5′ end, and which may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′) G, commonly written as m7GpppG. A cap species may also be an ARCA. A non-limiting list of possible cap species includes m7GpppG, m7Gpppm7G, m73′dGpppG, iri27′03′GpppG, iri27′03′GppppG, iri27′02′GppppG, m7Gpppm7G, m73′dGpppG, iri27′03′GpppG, iri27′03′GppppG, and m27 02′GppppG. An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. An mRNA may instead or additionally include a stem loop, such as a histone stem loop. A stem loop may include 1, 2, 3, 4, 5, 6, 7, 8, 9 or more nucleotide base pairs. For example, a stem loop may include 4, 5, 6, 7, 8, 9 nucleotide base pairs. A stem loop may be located in any region of an mRNA. For example, a stem loop may be located in, before, or after a UTR (a 5′ UTR or a 3′ UTR), a coding region, or a polyA sequence or tail. An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ UTR of an mRNA. The modified mRNA used in the present disclosure may comprise in addition to the coding region (which codes for the fusion protein and which may be codon-optimized) one or more of a stem loop, a chain terminating nucleoside, miRNA binding sites, a polyA sequence, a polyadenylation signal, 3′ and/or 5′ UTRs and/or a 5′ cap structure. As abovementioned, various nucleotide modifications are preferably incorporated into the mRNA to modify it for increased translation, reduced immunogenicity, and increased stability. Suitable modified nucleotides include but are not limited to N1-methyladenosine (m1A), N6-methyladenosine (m6A), 5-methylcytidine (m5C), 5-methyluridine (m5U), 2-thiouridine (s2U), 5-methoxyuridine (5moU), pseudouridine (ψ), N1-methylpseudouridine (m1ψ). Among these mRNA modifications, m5C and w are the most preferred as they reduce the immunogenicity of mRNA as well as increase the translation efficiency in vivo. In preferred embodiments of the present disclosure, the composition herein comprises a non-viral delivery vector such as an LNP or a liposome comprising a modified mRNA as the polynucleotide cargo, wherein the mRNA is modified with at least 50% m5C and 50% ψ or m1ψ, preferably at least 75% m5C and 75% ψ or m1ψ, and even more preferably 90% m5C and 90% ψ or m1ψ, or even more preferably 100% modification using m5C and ψ or m1ψ.

Such modified mRNAs preferably code for fusion proteins comprising (i) an EV polypeptide such as a tetraspanin (for instance CD63, CD81, CD9), PTGFRN, or Lamp2, (ii) a self-cleaving polypeptide domain, for instance a cis-cleaving domain, such as an intein, and (iii) a protein of interest (POI) in the form of an enzyme which is deficient in a disease selected from the inborn errors of metabolism, e.g. PAH, ASL, ASS, GAA, GLA, etc. In another preferred embodiment, the components (i), (ii) and (iii) can be further combined with (iv) a targeting entity expressed on the external surface of the engineered EV, thereby directing delivery to a preferred target cell and/or tissue, and (v) a polypeptide domain which binds to serum albumin, to further extend the already long half-life of the engineered EV comprising the POI.

As used herein, “polynucleotide” includes, for instance, cDNA, RNA, DNA/RNA hybrid, antisense RNA, siRNA, mRNA, shRNA, saRNA, doggy-bond DNA, ribozyme, genomic DNA, synthetic forms, and mixed polymers, both sense and antisense strands, and may be chemically or biochemically modified to contain non-natural or derivatised, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

Cargo may include nucleic acids such as siRNAs which target oncogenes known to be involved with the development of cancer. The genes targeted by the nucleic acids according to the present disclosure may be ABL, AF4/HRX, AKT-2, ALK, ALK/NPM, AML1, AML1/MTG8, AXL, BCL-2, 3, 6, BCR/ABL, c-MYC, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HER2/neu, HOX11, HST, IL-3, INT-2, JUN, KIT, KS3, K-SAM, LBC, LCK, LMO1, LMO2, L-MYC, LYL-1, LYT-10, LYT-10/Cα1, MAS, MDM-2, MLL, MOS, MTG8/AML1, MYB, MYH11/CBFB, NEU, N-MYC, OST, PAX-5, PBX1/E2A, PIM-1, PRAD-1, RAF, RAR/PML, RAS-H, RAS-K, RAS-N, REL/NRG, RET, RHOM1, RHOM2, ROS, SKI, SIS, SET/CAN, SRC, TAL1, TAL2, TAN-1, TIAM1, TSC2, TRK.

### Viral Cargos, Such as:

Exemplary viral cargos include: a viral vector which is an adeno-associated viral (AAV) vector or a lentiviral vector.

In some embodiments, the viral vector is an AAV vector. In some embodiments, the AAV vector comprises a capsid from human AAV serotype AAV1, AAV2, AAV3, AAV4, AAV5, AAV6, AAV7, AAV8, AAV9, AAV 10, AAV11 or AAV12. In some embodiments, the AAV vector comprises an AAV viral genome comprising inverted terminal repeat (ITR) sequences from human AAV serotype AAV1, AAV2, AAV3, AAV4, AAV5, AAV6, AAV7, AAV8, AAV9, r AAV10. In some embodiments, the AAV capsid and the AAV ITR are from the same serotype or from different serotypes.

In some embodiments of the above aspects and embodiments, the viral vector is a lentiviral vector. In some embodiments, the lentiviral vector is derived from HIV, a simian immunodeficiency virus or a feline immunodeficiency virus. In some embodiments, the lentiviral vector is non-replicating. In some embodiments, the lentiviral vector is non-integrating.

In some embodiments the viral vector comprises a viral capsid and a viral genome, the viral genome comprising one or more heterologous transgenes. In preferred embodiments, the heterologous transgene encodes a polypeptide or protein. The protein encoded with in the viral genome may be any one of the cargos according to the disclosure allowing the viral cargo to act as a gene replacement therapy.

In some embodiments the cargo loaded may additionally comprise one or more molecules that provide immune effector functions. Immune effector molecules are particularly useful in the case of EVs loaded with a viral (e.g., AVV or lentiviral) cargo but may equally be used where the EV is loaded with any cargo according to the disclosure. The immune effector may act to reduce immunogenicity of the EV. In some embodiments, the immune effector functions to stimulate immune inhibitors. In other embodiments, the immune effector functions to inhibit immune stimulating molecules. In some embodiments, EV comprises molecules that stimulate immune inhibitors and molecules that inhibit immune stimulating molecules.

Exemplary immune effector molecules include, but are not limited to, one or more of CTLA4, B7-1, B7-2, PD-I, PD-L1, PD-L2, CD28, or VISTA. In some embodiments, the envelope comprises CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-LI and VISTA.

The immune effector molecule may form part of the protein construct where the protein construct is optionally an EV protein or alternatively an endosomal escape enhancer (as EV protein substitute in addition to the endosomal escape function), the cargo loading construct, the targeting construct or may form part of an entirely separate fusion protein construct comprising an immune effector molecule fused to any EV protein according to the present disclosure.

### Specific Small Molecule Cargos, Such as:

anticancer agents such as doxorubicin, methotrexate, 5-fluorouracil or other nucleoside analogues such as cytosine arabinoside, proteasome inhibitors such as bortezomib, or kinase inhibitors such as imatinib or seliciclib, or NSAIDs such as naproxen, aspirin, or celecoxib, antibiotics such as heracillin, or antihypertensives such as ACE inhibitors such as enalapril, ARBs such as candesartan, etc. The present disclosure is naturally applicable also to other small molecules without departing from the gist of the disclosure.

In certain embodiments the cargo may be present on the inside of the EV, on the outside of the EV or in the membrane of the EV. The desired location of the cargo will depend on the nature of the cargo and its mechanism of action, for instance a membrane protein will preferably be located in the membrane of the EV, a decoy receptor will preferably be present on the surface of the EV but a cargo designed to be delivered into the cytosol or nucleus of the recipient cell, such as a silencing RNA will preferably be located inside the lumen of the EV.

The cargo may be loaded passively into the EVs by the cargo being present in the cytosol of the EV producing cells. Such passive loading applies, for instance, to nucleic acids, small molecules, viruses, soluble proteins or membrane proteins that are naturally loaded into the EVs. This is described in further detail below.

In certain embodiments the cargo is actively loaded into the EVs of the present disclosure, e.g., by EV protein or by endosomal escape enhancer. One form of active loading of cargos involves exogenous active loading which involves cargo being loaded using any known exogenous loading method including: electroporation, transfection with transfection reagents such a cationic transfection agents, Lipofectamine®, conjugation of the cargo to a membrane anchoring moiety such as a lipid or cholesterol tail or loading by means of a CPP, either in the form of a CPP-cargo conjugate or in the form of a CPP-cargo non-covalent complex. Again, this type of active loading may result in the cargo being located on the inside of the EV, on the outside of the EV or located within the membrane of the EV.

In certain embodiments the cargo is actively loaded into the EVs of the present disclosure by the use of fusion proteins, with an EV protein or alternatively an endosomal escape enhancer. In this case the cargo carried by the EV may form part of the endosomal escape enhancer-protein construct (preferably as a fusion protein), or alternatively the cargo carried by the EV forms part of an additional protein construct (preferably as a fusion protein) with an EV-protein separate to the with the endosomal escape enhancer. In either case the cargo protein may be fused into the fusion protein such that it is located on the inside of the EV, on the outside of the EV or located within the membrane of the EV. The presence of the EV protein in the fusion protein actively loads the therapeutic protein into the EV. The cargo protein may be engineered to be fused to a single or multi-pass transmembrane protein at either the C or N terminus to display the therapeutic protein on the surface of the EV or protect the cargo within the EV. Any EV protein, as defined above, may be employed as a fusion partner for loading cargos. EV proteins which may be employed to load cargo into EVs may be transmembrane but need not be.

The inventors have surprisingly found that the present disclosure is particularly useful in the delivery of bioactive cargo and other cargo that would benefit from being loaded into the lumen of the EV and the protective shell that it provides.

One or more of either the endosomal escape enhancer or the cargo may independently form part of a fusion protein with an EV protein and/or a release system, preferably a self-cleaving protein such as intein.

Optionally the EV is an exosome and the EV protein is an exosomal protein.

In a second aspect of the disclosure there is provided at least two plasmids, namely a first plasmid and a second plasmid. Each plasmid comprises at least one polynucleotide construct. One polynucleotide construct (a.k.a., the first) of the first plasmid encodes a protein construct that comprises an endosomal escape enhancer (e.g., VSV-G). The other polynucleotide construct (a.k.a. the second) of the second plasmid encodes a protein construct comprising a cargo, an EV protein and optionally a release system, preferably a self-cleaving protein such as intein. The protein construct comprising a cargo, an EV protein and optionally a release system may be as described for the first aspect of the disclosure.

The inventors have surprisingly found that when more than one plasmid may be needed the resulting protein constructs (VSV-G and EV-Cargo-[optional release system], preferably as a fusion protein) co-localise on the same EV (or exosome), which would not normally be expected.

Furthermore, the inventors have surprisingly found that in a population of EVs, each EV in the population comprise both the VSV-G and EV-cargo, and where a release system is used the release system, preferably as a fusion protein, in the EV membrane of the same EV.

It will be appreciated that a population of EVs where one EV in the population has an EV protein that is loaded with cargo and another EV in the population has the endosomal escape enhancer would not impart the benefits afforded by the combination of features (endosomal escape enhancer and cargo) as taught herein. In this scenario, a more likely output would be a mix of EVs containing the cargo but failing to escape the endo-lysosomal system and EVs that escape the endo-lysosomal system but contain no cargo and thus cannot deliver any cargo to the cytosol of a cell.

Simply adding two polynucleotides to the same cell will not guarantee that an EV will have both protein constructs, encoded by said polynucleotide constructs, within the same EV that leads to the effect, without further engineering.

Alternatively, there is provided one plasmid comprising at least one polynucleotide construct that encodes a protein construct that may comprise an endosomal escape enhancer such as VSV-G, optionally a multimerization domain (such as Foldon) and optionally a release system (preferably a self-cleaving protein such as intein). Optionally the protein construct is a fusion protein. The protein construct comprising an endosomal escape enhancer, optionally a multimerization domain and optionally a release system may be as described for alternative embodiments of the first aspect of the disclosure.

One advantage is that only one plasmid is required such that the potential risks of protein misfolding, toxic protein aggregation, translational error increases, imperfect association/dissociation between the proteins are avoided.

In a third aspect of the disclosure there is provided a cell comprising at least the plasmid(s) according to the second aspect or alternative thereto.

Generally, EVs may be derived from essentially any cell source, be it a primary cell source or an immortalized cell line. The EV source cells may be any embryonic, fetal, and adult somatic stem cell types, including induced pluripotent stem cells (iPSCs) and other stem cells derived by any method, as well as any adult cell source. The source cells for producing EVs of the present invention may be selected from a wide range of cells and cell lines, for instance mesenchymal stem or stromal cells (obtainable from e.g. bone marrow, adipose tissue, Wharton's jelly, perinatal tissue, chorion, placenta, tooth buds, umbilical cord blood, skin tissue, etc.), fibroblasts, amnion cells and more specifically amnion epithelial cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, erythrocytes, erythroid progenitors, chondrocytes, mesenchymal stromal cells (MSCs) of different origin, amnion cells, amnion epithelial (AE) cells, CEVEC's CAP® cells, any cells obtained through amniocentesis or from the placenta, airway or alveolar epithelial cells, fibroblasts, endothelial cells, etc. Also, immune cells such as B cells, T cells, NK cells, macrophages, monocytes, dendritic cells (DCs) are also within the scope of the present invention, and essentially any type of cell which is capable of producing EVs is also encompassed herein. The source cell may be either allogeneic, autologous, or even xenogeneic in nature to the patient to be treated, i.e. the cells may be from the patient himself or from an unrelated, matched or unmatched donor.

Advantages include, but are not limited to the following:
The single polynucleotide construct is smaller in size meaning greater stability of the polynucleotide construct in a cell and/or EV and lower chances of the polynucleotide construct from resulting in protein misfolding, toxic protein aggregation, translational error increases, imperfect association/dissociation between the proteins are avoided.
Multiple desirable functions are still achievable, e.g., the resultant protein construct will be capable of both endosomal escape and delivery of a free and unconjugated form of cargo.

Where two plasmids as herein disclosed are transfected into a host cell, the polynucleotide constructs are stably integrated into the host cell genome and a double-stable cell is achievable. The inventor's postulate that it is due to this stable transfection that may lead to the co-localisation of the corresponding protein constructs in the same EV that is observed.

Advantages include, but are not limited to the following:
polynucleotide construct is smaller in size meaning greater stability of the polynucleotide construct in a cell and lower chances of the protein construct from dissociation from the membrane.

Another advantage may include increased expression levels. Especially where you must use IRES or 2a poly peptides for expressing two proteins from one plasmid.

In a fourth aspect of the disclosure there is provided a protein construct, for use in a nanoparticle (such as an EV, preferably an exosome). The protein construct comprises an endosomal escape enhancer such as VSV-G, a multimerization domain (such as Foldon) and optionally a release system (preferably a self-cleaving protein such as intein). Optionally the protein construct is a fusion protein.

One advantage is that the singular protein construct having both an endosomal escape enhancer and the cargo and optional release system in the same protein construct enables the engineered EV to be simplified (i.e., by not having multiple protein constructs in the same EV) while still retaining the desired characteristics of endosomal escape and cytosolic delivery of a free unconjugated, and thus functionally active, cargo.

It will be appreciated that, in this embodiment, the producer cells are also simplified in that only a singular polynucleotide construct is required to be transfected into the host cell. Typically, such a method, using one vector (e.g., plasmid) is considered more stable than a method that uses two vectors (e.g., plasmids) and associated downstream risks of protein misfolding, toxic protein aggregation, and translational errors, imperfect association and dissociation between the proteins, may be avoidable.

Another advantage is the improved stability of the EVs (or exosomes), as there is less disturbance of the EV membrane (or exosomal membrane) and the other components contained therein (for example the [natural] protein and lipid profile).

Use of a single protein construct, preferably as a fusion protein, as herein disclosed, particularly VSVG-Cargo (and optionally release system and multimerization domain), also frees up more space on the EV membrane surface (or outer leaflet). This enables secondary protein construct(s) with, for example, a targeting moiety or protective moiety, to be introduced into the same EV while minimising the issue of steric hinderance between each protein construct.

It will be appreciated that, in this embodiment, the use of a singular polynucleotide construct encoding the abovementioned protein construct (VSVG-Cargo, and optionally release system and multimerization domain) means that a further polynucleotide construct may be transfected into the host cell that encodes the secondary protein construct(s) with, for example, a targeting moiety or protective moiety.

In a fifth aspect of the disclosure there is provided a method of making an engineered EV according to the first aspect, comprising the steps of: (i) introducing into an EV-producing cell a polynucleotide construct encoding an endosomal escape enhancer, (ii) expressing the polynucleotide construct in the EV-producing cell, and (iii) loading a cargo into the EV, thereby generating an EV comprising both an endosomal escape enhancer and a cargo. The endosomal escape enhancer may be a VSV-G protein construct.

In certain embodiments, the polynucleotide construct encodes a single protein construct comprising the endosomal escape enhancer and cargo. The polynucleotide construct may encode a single protein construct comprising an endosomal escape enhancer and one or more of a multimerization domain, a release system and a cargo. Preferably the protein construct is a fusion protein.

Alternatively, two polynucleotides constructs may be introduced, one encoding a protein construct having the cargo and the other encoding a protein construct having the endosomal escape enhancer in a separate construct to the cargo.

In this alternative, a double stable cell and/or EV is achieved. Where two polynucleotide constructs are introduced, the resulting protein construct having the cargo may also comprise an EV protein. Preferably the protein construct is a fusion protein.

Each of the endosomal escape enhancer, such as VSV-G protein, and/or the cargo may be independently displayed on the surface of the EV and/or may be loaded into the lumen of the EV. It will be appreciated that each polynucleotide construct is introduced independently of one another. Consequently, the constructs may be introduced in any order and/or may be introduced in parallel to one another (e.g., at the same time).

In a particular embodiment, there is provided a method of making an EV according to the first aspect, comprising:
(i) introducing into an EV-producing cell a first polynucleotide construct encoding an EV protein, optionally a release system, and a cargo, preferably wherein the first polynucleotide construct encodes a fusion protein comprising the EV protein, the optional release system, and the cargo;
(ii) introducing into the EV-producing cell a second polynucleotide construct encoding an endosomal escape enhancer (e.g., VSV-G), and
(iii) culturing the EV-producing cell under conditions to generate an EV comprising the endosomal escape enhancer, the EV protein, the optional release system, and the cargo. In embodiments involving a fusion protein, the resultant EV comprises the fusion protein and the endosomal escape enhancer, as expressed by the first and the second polynucleotide constructs.

In an alternative embodiment, there is provided a method of making an EV according to the first aspect, comprising:
(i) introducing into an EV-producing cell a polynucleotide construct encoding an endosomal escape enhancer (e.g., VSV-G), optionally a multimerization domain (e.g., Foldon), optionally a release system (e.g., intein), and a cargo; preferably as a fusion protein; and
(ii) culturing the EV-producing cell under conditions to generate an EV comprising the endosomal escape enhancer, the optional multimerization domain, the optional release system, and the cargo. In embodiments involving a fusion protein, the resultant EV comprises the fusion protein.

Particularly advantageous is when the cargo is loaded into the lumen of the cell and the endosomal escape enhancer is displayed on the surface of the EV (or exosome).

One advantage is that steric hindrance is avoided, and better loading is provided. In this embodiment the release system, preferably self-cleaving protein such as intein is particularly helpful.

The inventors have surprisingly found a release system to be especially useful in combination with the cargo and an endosomal escape enhancer. The inventors have postulated that release of the cargo, in unconjugated form, into the lumen of the EV (or exosome) means that the cargo does not compete with the endosomal escape enhancer displayed on the surface of the EV. The lack of steric hindrance meaning that both the cargo and the endosomal escape components are more effective in their roles.

The method for producing the EVs comprises a step of loading the EV with at least one cargo. Said cargo loading step as hereinabove may be by endogenous loading. Where the cargo is loaded by endogenous loading, the cargo is either loaded by the same fusion construct as the endosomal escape enhancer or the cargo is loaded by a second polynucleotide construct that encodes a Cargo-EV protein construct, preferably a fusion protein.

The method for producing the EVs may further comprise a step of loading the EV with one or more additional functional components, the functional component(s) being selected from a further cargo, a targeting moiety and/or a protective moiety.

Where the loading step is an exogenous loading step, the loading step may comprise loading of the functional component by any exogenous loading method including electroporation, microfluidics, transfection with transfection reagents such a cationic transfection agents, Lipofectamine®, conjugation of the cargo to a membrane anchoring moiety such as a lipid or cholesterol tail or loading by means of a CPP, either in the form of a CPP-cargo conjugate or in the form of a CPP-cargo non-covalent complex. Where the loading step is endogenous, the endogenous loading step may comprise introducing the functional component into the same polynucleotide construct as the cargo or endosomal escape enhancer or introducing into the EV-producing cell a further polynucleotide construct encoding the functional component.

It is to be appreciated that a single nucleic construct might encode separately both the protein constructs as per the present disclosure as well as the functional component using a bidirectional plasmid. Said functional component construct may simply be expressed by the EV-producing cell and passively loaded into the EVs or the functional component may be comprised as a separate protein construct so that the functional component, when translated, is endogenously and actively loaded into the EVs produced by the EV-producing cell.

Without wishing to be bound by theory, the benefit of generating the cells as double or multiple stable cells, as hereinabove, is that a large library of producer cell lines can be generated quickly and easily by swapping out the cargo and/or targeting construct. Additionally, each separate construct can be placed under the control of a different promoter and thus the expression levels of the albumin, cargo and/or targeting moiety can be carefully and individually controlled. Alternatively, when the therapeutic cargo and/or the targeting moiety forms part of the albumin-EV protein fusion construct, the advantage of a single albumin-EV protein-cargo/targeting-moiety construct is that it only requires the cells to be made single stable, which results in the generation of simple and robust cell lines. The choice of a single, double, or multiple stable cell line will depend upon the cargo and targeting moieties desired, their size and desired location on the EV.

In certain preferred embodiments of the above method the endosomal escape enhancer includes, but is not limited to, VSV-G and the release system includes, but is not limited to, intein.

It will be appreciated that the EV as herein described may form part of a population of EVs as herein defined. For example, one population may comprise several EVs, each EV having two constructs, for example, the EV protein-release system-cargo construct+endosomal escape enhancer construct in common. A collection of EVs may comprise EVs belonging to the same or different populations as well as EVs that may not fall into any particular population having the desired protein constructs as herein disclosed.

The EVs of the present invention, in some embodiments, are isolated EVs. Accordingly, the present invention provides isolated EVs according to the present disclosure.

In a sixth aspect of the disclosure there is provided an in vitro method for assaying engineered EVs according to the first aspect of the disclosure. The method comprises the steps of: (i) co-culturing reporter cells and EV-producing cells capable of producing the engineered EVs, and (ii) measuring signal-positive cells. Signal-positive cells can be measured using FACS.

The reporter cells may comprise nucleic acid sequences encoding fluorescent proteins, wherein the fluorescent proteins act as reporters. The reporter cells may be traffic light reporter cells. A suitable fluorescent protein to act as reporter is GFP.

The cell type of the reporter cells may include Hela, T47D, Hepg2, B16F10 and/or any other cell-line modified to function as a traffic light reporter cell. Preferably the traffic light reporter cells are B16F10.

The reporter cells may comprise an above average receptor level expression, preferably VSV-G (LDL-R) receptor level expression. Preferably, the above average receptor level expression is from at least about 51%, more preferably at least about 55%, more preferably at least about 65%, 70%, 75%, 80%, 85%, 90% and most preferably at least about 95%, of the overall expression profile.

The EV-producing cells, preferably exosome-producing cells, may include HEK293T cells.

The method may have a delivery saturation between a cell-to-cell ratio of from about 1:5 to about 1:1 EV-producing cells to reporter cells.

The method may have a detection limitation at a cell-to-cell ratio of at least about 30:1 EV-producing cells to reporter cells, and preferably from about 30:1 to about 50:1 EV-producing cells to reporter cells, more preferably when the reporter cell is selected from Hela and/or T47D. Most preferably the method has a detection limitation between a cell-to-cell ratio of from at least about 50:1 when the reporter cells are B16F10.

In a seventh aspect of the disclosure there is provided a composition comprising an EV according to the first aspect and an excipient, diluent, vehicle, solvent and/or carrier.

Exemplary excipients, diluents, vehicles, solvents and/or carriers, which may be used in the context of the present disclosure include, but are not limited to the following non-limiting examples: degradation or loss of activity stabiliser excipients such as proteins such as HSA, polyols such as glycerol, sorbitol and erythritol, amino acids such as arginine, aspartic acid, glutamic acid, lysine, proline, glycine, histidine and methionine, polymers such as polyvinylpyrrolidone and hydroxypropyl cellulose, surfactants such as polysorbate 80, polysorbate 20 and pluronicF68, antioxidants such as ascorbic acid and alpha-tocopherol (vitamin E), buffers such as acetate, succinate, citrate, phosphate, histidine, tris(hydroxymethyl)aminomethane (TRIS), metal ion/chelators such as Ca²⁺, Zn²⁺ and EDTA, Cyclodextrin based such as hydroxypropyl β-cyclodextrin and others such as polyanions and salts, stabilisers or bulking agents such as lactose, trehalose, dextrose, sucrose, sorbitol, glycerol, albumin, gelatin, mannitol and dextran, or preservatives such as benzyl alcohol, m-cresol, phenol, 2-phenoxyethanol or any other excipient, diluent, vehicle, solvent and/or carrier known at the time of the present disclosure.

The composition may be a pharmaceutical composition in which the excipient, diluent, vehicle, solvent or carrier is pharmaceutically acceptable.

Exemplary pharmaceutically acceptable excipients, diluents, vehicles, solvents and/or carriers, which may be used in the context of the present disclosure include, but are not limited to, the abovementioned non-limiting examples, wherein said excipients, diluents, vehicles, solvents and/or carriers are considered pharmaceutically acceptable, or any other pharmaceutically acceptable excipients, diluents, vehicles, solvents and/or carriers known at the time of the present disclosure.

The composition may be suitably formulated for administration human or animal subject via various different administration routes including, but are not limited to the following non-liming examples: auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the EVs, the cargo molecule in question, or the EV population as such.

The composition may comprise a population of EVs according to the first aspect.

One advantage of the compositions and formulations as hereinabove described are that the EVs are packaged in a suitable format for effective delivery to a subject, preferably a mammal, more preferably a human.

Typically, EVs (e.g., exosomes) will be administered as a population of EVs (or exosomes).

In this embodiment of the disclosure the pharmaceutical composition and/or dosage comprises a population of EVs according to the disclosure in which the average number of cargo molecules per EV is above or below one cargo molecule per EV. In another embodiment, in the population of EVs according to the disclosure, at least 5%, at least 10%, at least 20%, at least 50%, at least 70%, at least 75%, at least 80%, at least 85%, at least 90%, and/or at least 95% of all EVs comprise at least one endosomal escape enhancer and at least one cargo.

It will be appreciated that a population of EVs which may comprise thousands, millions, billions or even trillions of EVs. EVs may be present in concentrations such as 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume or per unit of weight (for instance per ml or per L or per kg of body weight), or any other number larger, smaller, or anywhere in between. In the same vein, the term “population”, which may e.g., relate to an EV comprising a certain cargo shall be understood to encompass a plurality of entities constituting such a population.

In an eighth aspect of the disclosure there is provided either an EV according to the first aspect, or a composition according to the seventh aspect for use as a medicament, such as in the treatment of cancer and/or brain-related conditions, disorders and/or diseases. Preferably, a condition/disorder/disease in which gene-editing and/or gene silencing would be beneficial. In a related aspect there is provided a method of treatment wherein either an EV according to the first aspect, or a composition according to the seventh aspect is administered to a subject, such as in the treatment of cancer and/or brain-related conditions, disorders and/or diseases.

In a ninth aspect of the disclosure there is provided use of either an EV according to the first aspect, or a composition according to the seventh aspect for gene editing/gene silencing. In a related aspect of the disclosure there is provided an in vitro method for editing and/or silencing a target gene in a cell and/or cell line using an EV according to the first aspect, or a composition according to the seventh aspect. It will be appreciated that said in vitro method may have diagnostic applications.

The medical use or method of treatment may be by delivery of any kind of cargo according to the present disclosure. For instance, the medical use or treatment may be by delivery of functional proteins as protein replacement therapy, delivery of mRNA encoding functional proteins to also act as a protein replacement therapy. Such a protein replacement therapy may, for instance, be ERT for diseases caused by inborn errors in metabolism, such as phenylketonuria (PKU), urea cycle disorders, or lysosomal storage disorders. The medical use or treatment may be by delivery of gene silencing RNAs, splice switching RNAs, or CRISPR-Cas9 for gene editing.

The medical use or treatment may be gene therapy by delivery of plasmid DNA, mini-circles or viral gene therapies such as AAVs or lentiviruses. The medical use or treatment may be by presentation of an antigen or neoantigen for immunotherapy, in effect acting as a vaccine to induce an immune response. For instance, the EV may act by delivery and/or presentation of a tumour antigen for cancer immunotherapy, or viral, bacterial or fungal antigens for immunization against pathogens. The medical use or treatment may be by delivery of small molecules, biologics, antibodies or antibody-drug conjugates capable of mediating a therapeutic effect once delivered into a cell or the extracellular matrix. In one embodiment, the medical use or treatment may be affected by the EVs comprising more than one type of bioactive cargo, i.e., the bioactive cargo may be a mixture of protein, nucleic acid, virus, viral genome, antigen, small molecule and/or biologic.

Importantly, the present invention relates to use of the EVs or pharmaceutical composition described herein in the prophylaxis and/or treatment and/or alleviation of a variety of diseases, typically via the delivery of essentially any type of bioactive cargo, such as drug cargo. For instance: a nucleic acid such as an RNA molecule, a DNA molecule or a mixmer, mRNA, antisense or splice-switching oligonucleotides, siRNA, shRNA, miRNA, pDNA, supercoiled or unsupercoiled plasmids, mini-circles, peptides or proteins including transporters, enzymes, receptors such as decoy receptors, membrane proteins, cytokines, antigens and neoantigens, ribonuclear proteins, nucleic acid binding proteins, antibodies, nanobodies, antibody fragments, antibody-drug conjugates, small molecule drugs, gene editing technology such as CRISPR-Cas9, TALENs, meganucleases, or vesicle-based cargos such as viruses (e.g. AAVs, lentiviruses, etc.). The cargo may be a mixture of protein, nucleic acid, virus, viral genome, antigen, small molecule and/or biologic.

Non-limiting examples of diseases and conditions that are suitable targets for treatment using the EVs and pharmaceutical compositions described herein include the following non-limiting examples: autoimmune diseases (such as celiac disease, Crohn's disease, diabetes mellitus type 1, Graves' disease, inflammatory bowel disease, multiple sclerosis, psoriasis, rheumatoid arthritis, systemic lupus erythematosus, ulcerative colitis, ankylosing spondylitis, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis), stroke, acute spinal cord injury, vasculitis, Guillain-Barre syndrome, acute myocardial infarction, acute respiratory distress syndrome (ARDS), sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, haemophilia type A, B or C, Duchenne muscular dystrophy (DMD) and other muscular dystrophies, in-born errors of metabolism including disorders of carbohydrate metabolism, e.g., G6PD deficiency galactosemia, hereditary fructose intolerance, fructose 1,6-diphosphatase deficiency and the glycogen storage diseases, disorders of organic acid metabolism (organic acidurias), such as alkaptonuria, 2-hydroxyglutaric acidurias, methylmalonic or propionic acidemia, multiple carboxylase deficiency, disorders of amino acid metabolism, such as PKU, maple syrup urine disease, glutaric acidemia type 1, aminoacidopathies, e.g., hereditary tyrosinemia, nonketotic hyperglycinemia, and homocystinuria, hereditary tyrosinemia, Fanconi syndrome, Primary Lactic Acidoses e.g., pyruvate dehydrogenase, pyruvate carboxylase and cytochrome oxidase deficiencies, disorders of fatty acid oxidation and mitochondrial metabolism, such as short, medium, and long-chain acyl-CoA dehydrogenase deficiencies also known as Beta-oxidation defects, Reye's syndrome, medium-chain acyl-coenzyme A dehydrogenase deficiency (MCADD), mitochondrial encephalopathy lactic acidosis and stroke-like episodes (MELAS), myoclonic epilepsy with ragged red fibers (MERRF), pyruvate dehydrogenase deficiency, disorders of porphyrin metabolism such as acute intermittent porphyria, disorders of purine or pyrimidine metabolism such as Lesch-Nyhan syndrome, disorders of steroid metabolism such as lipoid congenital adrenal hyperplasia, congenital adrenal hyperplasia, disorders of mitochondrial function such as Kearns-Sayre syndrome, disorders of peroxisomal function such as Zellweger syndrome and neonatal adrenoleukodystrophy, congenital adrenal hyperplasia or Smith-Lemli-Opitz, Menkes syndrome, neonatal hemochromatosis, urea cycle disorders such as N-acetylglutamate synthase deficiency, carbamoyl phosphate synthetase deficiency, OTC deficiency, citrullinemia (deficiency of argininosuccinic acid synthase), argininosuccinic aciduria (ASA; deficiency of argininosuccinic acid lyase), argininemia hyperornithinemia, hyperammonemia, (deficiency of arginase), homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1), orotic aciduria (deficiency in the enzyme, UMPS), all of the lysosomal storage diseases, for instance alpha-mannosidosis, beta-mannosidosis, aspartylglucosaminuria, cholesteryl ester storage disease, cystinosis, Danon disease, Fabry disease, Farber disease, fucosidosis, galactosialidosis, Gaucher disease Type I, Gaucher disease Type II, Gaucher disease Type III, GM1 gangliosidosis Type I, GM1 gangliosidosis Type II, GM1 gangliosidosis Type III, GM2-Sandhoff disease, GM2—Tay-Sachs disease, GM2-gangliosidosis, AB variant, mucolipidosis II, Krabbe disease, lysosomal acid lipase deficiency, metachromatic leukodystrophy, MPS I—Hurler syndrome, MPS I—Scheie syndrome, MPS I—Hurler-Scheie syndrome, MPS II—Hunter syndrome, MPS IIIA—Sanfilippo syndrome Type A, MPS IIIB—Sanfilippo syndrome Type B, MPS IIIB—Sanfilippo syndrome Type C, MPS IIIB—Sanfilippo syndrome Type D, MPS IV Morquio Type A, MPS IV—Morquio Type B, MPS IX—hyaluronidase deficiency, MPS VI—Maroteaux-Lamy, MPS VII—Sly syndrome, Mucolipidosis I—sialidosis, mucolipidosis IIIC, mucolipidosis Type IV, mucopolysaccharidosis, multiple sulfatase deficiency, neuronal ceroid lipofuscinosis T1, neuronal ceroid lipofuscinosis T2, neuronal ceroid lipofuscinosis T3, neuronal ceroid lipofuscinosis T4, neuronal ceroid lipofuscinosis T5, neuronal ceroid lipofuscinosis T6, neuronal ceroid lipofuscinosis T7, neuronal ceroid lipofuscinosis T8, neuronal ceroid lipofuscinosis T9, neuronal ceroid lipofuscinosis T10, Niemann-Pick disease Type A, Niemann-Pick disease Type B, Niemann-Pick disease Type C (NPC), Pompe disease, pycnodysostosis, Salla disease, Schindler disease and Wolman disease, etc., cystic fibrosis, primary ciliary dyskinesia, pulmonary alveolar proteinosis, arthrogryposis-renal dysfunction-cholestasis (ARC) syndrome, Rett syndrome, neurodegenerative diseases including Alzheimer's disease, Parkinson's disease, GBA associated Parkinson's disease, Huntington's disease and other trinucleotide repeat-related diseases, prion diseases, dementia including frontotemporal lobe dementia, amyotrophic lateral sclerosis (ALS), motor neuron disease, multiple sclerosis, cancer-induced cachexia, anorexia, diabetes mellitus type 2, and various cancers.

The present invention is advantageous for the treatment of cancers; and especially advantageous, in the treatment of cancer by immunotherapy, due to accumulation in the lymph nodes.

Specifically, it is envisaged that the present invention is useful in the treatment of cancer by immunotherapy, i.e., the presentation of cancer antigens on the surface of albumin EVs so that those antigens raise an immune response against the cancer antigen. Virtually all types of cancer are relevant disease targets for the present invention, for instance, acute lymphoblastic leukemia (ALL), acute myeloid leukemia, adrenocortical carcinoma, AIDS-related cancers, AIDS-related lymphoma, anal cancer, appendix cancer, astrocytoma, cerebellar or cerebral, basal-cell carcinoma, bile duct cancer, bladder cancer, bone tumor, brainstem glioma, brain cancer, brain tumor (cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumor (childhood, gastrointestinal), carcinoma of unknown primary, central nervous system lymphoma, cerebellar astrocytoma/malignant glioma, cervical cancer, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, extracranial germ cell tumor, extragonadal germ cell tumor, extrahepatic bile duct cancer, eye Cancer (intraocular melanoma, retinoblastoma), gallbladder cancer, gastric (stomach) cancer, gastrointestinal carcinoid tumor, gastrointestinal stromal tumor (GIST), germ cell tumor (extracranial, extragonadal, or ovarian), gestational trophoblastic tumor, glioma (glioma of the brain stem, cerebral astrocytoma, visual pathway and hypothalamic glioma), gastric carcinoid, hairy cell leukemia, head and neck cancer, heart cancer, hepatocellular (liver) cancer, Hodgkin lymphoma, hypopharyngeal cancer, intraocular melanoma, islet cell carcinoma (endocrine pancreas), Kaposi sarcoma, kidney cancer (renal cell cancer), laryngeal cancer, leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia)), lip and oral cavity cancer, liposarcoma, liver cancer (primary), lung cancer (non-small cell, small cell), lymphomas, AIDS-related lymphoma, Burkitt lymphoma, cutaneous T-cell lymphoma, Hodgkin lymphoma, non-Hodgkin, medulloblastoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic/myeloproliferative diseases, yelogenous leukemia, chronic myeloid leukemia (acute, chronic), myeloma, nasal cavity and paranasal sinus cancer, nasopharyngeal carcinoma, neuroblastoma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic islet cell cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, pleuropulmonary blastoma, prostate cancer, rectal cancer, renal cell carcinoma (kidney cancer), retinoblastoma, rhabdomyosarcoma, salivary gland cancer, sarcoma (Ewing family of tumors sarcoma, Kaposi sarcoma, soft tissue sarcoma, uterine sarcoma), Sézary syndrome, skin cancer (nonmelanoma, melanoma), small intestine cancer, squamous cell, squamous neck cancer, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine cancer, uterine sarcoma, vaginal cancer, vulvar cancer, Waldenstrom macroglobulinemia, and/or Wilm's tumor.

It is envisaged that any dosage regime would be applicable to the EVs according to the present disclosure. The dosage regime chosen will depend on the cargo being delivered by the EVs of the present disclosure and the disease to be treated and any additional therapies being administered which will be determined by the skilled physician. The EV (or exosome) may be modified to display an endosomal escape enhancer (e.g., VSV-G) on the surface of the EV and the cargo in the lumen of the EV to improve release and delivery of the cargo at the desired site of action (e.g., the cytosol of a cell). Meaning that the frequency and schedule of repeat dosages may be modified and thus side-effects avoided and/or reduced.

It is envisaged that the EVs of the present disclosure will be administered multiple times, i.e., more than once, but normally more than two times or potentially for chronic, long-term treatment (i.e., administered tens to hundreds to thousands of times). Preferably, if the cargo is an antigen that is being administered as a vaccine, the immunization schedule will involve two or more administrations of the polypeptide, spread out over several weeks. Similarly, if the cargo is e.g., an RNA agent such as an siRNA or mRNA or a protein such as an antibody or an enzyme or a transporter, or a viral cargo such as an AAV or lentivirus, the EVs according to the present disclosure comprising the cargo in question will likely be administered more than once, normally multiple times as part of a chronic treatment regimen.

### Definitions

For convenience and clarity, certain terms employed herein are collected and described below. Unless otherwise defined, all technical and scientific terms used herein have the same meaning as commonly understood by one of ordinary skill in the art to which this disclosure belongs.

In the specification, the singular forms also include the plural unless the context clearly dictates otherwise; as examples, the terms “a,” “an,” and “the” are understood to be singular or plural and the term “or” is understood to be inclusive. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

“About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The term “at least one” as used herein can mean at least two, at least three, at least four, at least five, at least six, at least seven, at least eight, at least nine, at least ten or more.

The terms “extracellular vesicle” or “EV” are used interchangeably herein and can be understood to relate to any type of vesicle that is obtainable from a cell in any form. The size of EVs may vary considerably, but an EV typically comprises a volume defined by a bi-lipid membrane and having a nano-sized hydrodynamic radius, i.e., a radius below 1000 nm. The volume may comprise the vesicular secretome. The different types of EVs are defined by different morphologies, structure, messages and function. EVs can be broadly divided into two categories, (1) ectosomes and (2) exosomes. Some examples of EVs include for instance an exosome, an apoptotic body, arrestin domain containing protein 1 [ARRDC1]-mediated microvesicles (ARMMs), an ectosome, such as a microparticle or microvesicle, or a cardiosome, etc. Essentially, the terms ‘extracellular vesicle’ and/or ‘EV’ may relate to any type of lipid-based structure (with vesicular morphology or with any other type of suitable morphology) that can act as a delivery or transport vehicle or that has native therapeutic or pharmacological effects.

Furthermore, the said terms can be understood to also relate to, in some embodiments, extracellular vesicle mimics, cellular membrane vesicles obtained through membrane extrusion, sonication or other techniques, etc.

Clearly, EVs may be derived from any cell type, whether in vivo, ex vivo or in vitro (further details of suitable source or producer cells are herein described below).

Exosomes, microvesicles and ARMMs are just some examples of the different subtypes that fall under the hereinbefore broader description of EVs and represent particularly preferable EVs, but it will be appreciated that other EVs may also be advantageous in certain circumstances. Advantageously, the EV is an exosome.

The terms “apoptotic body” or “apoptotic bodies” are used interchangeably herein and can be understood to relate to any type of vesicle that is obtainable or derivable from apoptotic cells. Typically, an apoptotic body has a diameter ranging from about 1 μm to about 5 μm.

The terms “cardiosome” or “cardiosomes” are used interchangeably herein and can be understood to relate to any type of vesicle that is obtainable or derivable from cardiac cells.

The terms “ectosome” or “ectosomes” are used interchangeably herein and can be understood to relate to any type of heterogenous vesicle that is obtainable or derivable from outward budding of the plasma membrane and/or cell membrane of a cell, preferably from neutrophils and monocytes in serum.

Examples of ectosomes include, but are not limited to, microvesicles, microparticles and large vesicles. Typically, ectosomes range in size from about 50 nm to about 1 μm.

The terms “microparticle” or “microparticles” are used interchangeably herein and can be understood to relate to any type of vesicle that is obtainable or derivable from platelets.

The terms “microvesicle”, and “microvesicles” are used interchangeably herein and can be understood to relate to any type of vesicle that is obtainable or derivable or shed from the plasma membrane or cell membrane of a cell.

The term “ARMMs” can be understood to relate to any type of vesicle that is obtainable or derivable from the plasma membrane or cell membrane of a cell, from which they bud directly. Such microvesicles are mediated by the ARRDC1 and typically lack known late endosomal markers. As such, ARMMs are distinct from exosomes herein described.

The terms “exosome” or “exosomes” are used interchangeably herein and can be understood to relate to any type of vesicle that is obtainable or derivable from the endosomal, lysosomal and/or endo-lysosomal pathway and/or from inward budding of the plasma membrane and/or cell membrane. Exosomes often have a size of from about 30 to about 300 nm, typically in the range of from about 40 to about 250 nm, and sometimes from about 40 to about 160 nm, which is a highly suitable size range.

The term “modified” can mean that the vesicle has been modified either using genetic or chemical approaches, for instance via genetic engineering of the EV-producing cell, preferably an exosome-producing cell or via e.g., chemical conjugation, for instance to attach moieties to the EV, preferably the exosome surface.

The terms “genetically modified” and “genetically engineered” are used interchangeably herein and can mean that the EV, preferably an exosome, is derived from a genetically modified/engineered cell or is otherwise genetically engineered to express and/or modify the expression of proteins in the lumen, extravesicular membrane and/or displayed on the surface of the EV (e.g., exosome), which is typically incorporated into the EVs, preferably exosomes, produced by those cells.

Furthermore, the said terms shall be understood to also relate to, in some embodiments, EV mimics, cellular membrane vesicles obtained through membrane extrusion, sonication or other techniques, etc.

The terms “EV protein” “EV polypeptide”, “EV carrier protein” are used interchangeably herein and shall be understood to relate to any suitable protein naturally derived and/or expressed and enriched in an EV as herein defined. Certain proteins may be found in both exosomes and other EVs (e.g., ectosomes and apoptotic bodies) but are enriched in one set over another. The term shall be understood as comprising any polypeptide that enables transporting, trafficking, or shuttling of a fusion protein construct to a vesicular structure, such as an EV. Where embodiments relate to EVs it will understood that the corresponding EV proteins will apply. An EV protein as described herein can therefore be engineered to form one part of a fusion protein capable of transporting another part of the same fusion protein (here, an endosomal escape enhancer, optionally with a cargo and/or release system and/or targeting moiety) to the extravesicular membrane of an EV. Examples of EV proteins include transmembrane proteins, preferably multi-pass transmembrane protein and other hallmark EV membrane associated proteins, such as, but not limited to MMP2 and CK18.

The terms “exosome protein”, “exosomal protein”, “exosomal polypeptide”, “exosomal carrier protein” are used interchangeably herein and shall be understood to relate to any suitable protein naturally derived and/or expressed and enriched in an exosome compared to other vesicles/organelles/parent cell. The terms shall be understood as comprising any polypeptide that enables transporting, trafficking or shuttling of a fusion protein construct to an exosome. Where preferred embodiments relate to exosomes it will understood that the corresponding exosomal proteins will apply. An exosomal protein as described herein can therefore be engineered to form one part of a fusion protein capable of transporting another part of the same fusion protein (here, an endosomal escape enhancer, optionally with a cargo and/or release system and/or targeting moiety) to the extravesicular membrane of an exosome.

Examples of exosomal proteins include transmembrane proteins, preferably multi-pass transmembrane proteins and other hallmark exosomal membrane associated proteins, such as, but not limited to CD81, CD9 and CD63.

The terms “N-terminus”, “N terminal”, “N-terminal domain” and “NTD” are used interchangeably herein and shall be understood to be accorded the conventional meaning in the field unless otherwise indicated.

The terms “C-terminus”, C-terminal “, C-terminal domain” and “CTD” are used interchangeably herein and shall be understood to be accorded the conventional meaning in the field unless otherwise indicated.

An “endosomal escape enhancer” is a generally understood term in the art and may include any protein, (poly) peptide, or fragment, including, but not limited to, virally- and/or bacterially-derived components that enable, facilitate and/or enhance the release of an EV (e.g., exosome) and/or lysosome from the endo-lysosomal pathway and/or architecture and in doing so greatly increase the cytoplasmic delivery in vitro and in vivo. The endosomal escape enhancer may not exist naturally in an EV. Rather, the EV is engineered so as to contain the endosomal escape enhancer.

The term “self-cleaving protein” can mean a naturally occurring protein that excises itself from a native host protein through self-cleavage. A suitable example of a self-cleaving protein is an intein. It is to be appreciated that certain modifications are desirable to provide a protein that has self-cleaving capability only (i.e., no self-splicing). As suitable example of a protein capable of only self-cleavage (and no splicing) is ΔI-CM.

The term “self-splicing protein” can mean a naturally occurring protein that excises itself from a native host protein through self-splicing and ligation of their flanking peptide bonds. A suitable example of a self-splicing protein is an intein.

The terms “mini-intein” or “delta-intein” are used interchangeably herein and can be understood to relate to a modified intein, preferably from parent RecA, and lacking the endonuclease domain.

The term “Fast-cleaving intein” can be understood to relate to an intein or mini-intein that has been modified at the +C-extein position and/or —N-extein position so that the cleavage rate of said intein is quicker/faster than the original RecA, intein, or mini-intein.

The term “Slow-cleaving intein” can be understood to relate to an intein or mini-intein that has been modified at the +C-extein position and/or —N-extein position so that the cleavage rate of said intein is slower than the original RecA, intein, or mini-intein. An example of a slow-cleaving intein is ΔI-CM.

A “nucleic acid” can refer to a polynucleotide and includes polyribonucleotides and poly-deoxyribonucleotides. Nucleic acids according to the present disclosure may include any polymer or oligomer of pyrimidine and purine bases, e.g., cytosine (C), thymine (T) and uracil (U), and adenine (A) and guanine (G), respectively (see Albert L. Lehninger, Principles of Biochemistry, at 793-800 (Worth Pub. 1982) and G. Michael Blackburn, Michael J. Gait, David Loakes and David M. Williams, Nucleic Acids in Chemistry and Biology 3rd edition, (RSC publishing 2006), which are herein incorporated in their entirety for all purposes). Indeed, the present disclosure contemplates any deoxyribonucleotide or ribonucleotide component, and any chemical variants thereof. The polymers or oligomers may be heterogeneous or homogeneous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” can mean a nucleic acid ranging from at least 2, at least 8, at least 15 or at least 25 nucleotides in length, but may be up to 50, 100, 1000, 5000, 10000, 15000, or 20000 nucleotides long or a compound that specifically hybridises to a polynucleotide. Polynucleotides include sequences of DNA or RNA or mimetics thereof, which may be isolated from natural sources, recombinantly produced or artificially synthesised. A further example of a polynucleotide as employed in the present disclosure may be a peptide nucleic acid (PNA; see U.S. Pat. No. 6,156,501, which is hereby incorporated by reference in its entirety.) The disclosure also encompasses situations in which there is a non-traditional base pairing, such as Hoogsteen base pairing, which has been identified in certain tRNA molecules and postulated to exist in a triple helix. “Polynucleotide” and “oligonucleotide” are used interchangeably herein. It will be understood that when a nucleotide sequence is represented herein by a DNA sequence (e.g., A, T, G, and C), this also includes the corresponding RNA sequence (e.g., A, U, G, C) in which “U” replaces “T”.

The term “polynucleotide” includes, for instance, cDNA, RNA, DNA/RNA hybrid, antisense RNA, siRNA, mRNA, ribozyme, genomic DNA, synthetic forms, and mixed polymers, both sense and antisense strands, and may be chemically or biochemically modified to contain non-natural or derivatised, synthetic, or semi-synthetic nucleotide bases. Also contemplated are alterations of a wild-type or synthetic gene, including, but not limited to, deletion, insertion, substitution of one or more nucleotides, or fusion to other polynucleotide sequences.

The term “Pumilio and FBF proteins” or “PUF proteins” encompasses all related proteins and domains of such proteins (which may also be termed “PUM proteins”), for instance, human Pumilio homolog 1 (PUM1), PUMx2 or PUFx2, which are duplicates of PUM1, etc., or any NA-binding domains obtainable from any PUF (PUM) proteins.

The term “cargo” can mean any molecule that is physically and chemically able to be carried by an EV. It is suitably a molecule that does not naturally occur in an EV, i.e., the EV is engineered so as to contain the cargo. The term “cargo” shall be understood to relate to either a diagnostic cargo or a therapeutic cargo or any combination thereof. Diagnostic cargo and therapeutic cargo are hereinafter defined. The cargo is suitably bioactive cargo, also hereinafter defined.

The term “diagnostic cargo” shall be understood to relate to any chemical or biological molecule that might suitably be employed in a diagnostic application of the EVs according to the present disclosure. Examples of suitable diagnostic cargo include but are not limited to radiolabels, fluorescent labels, (bio) luminescent labels and reporters.

The term “therapeutic cargo” and “drug cargo” are used interchangeably herein and shall be understood to relate to any large chemical or biological molecule or small chemical or biological molecule designed for the treatment and/or prophylaxis of a condition, disease and/or disorder. Large molecules and small molecules are hereinafter defined.

The term “bioactive” can mean having a biological effect. Thus, with respect to diagnostic cargo, the term “bioactive” can mean any molecule that enables the identification of one or more properties of living matter. With respect to therapeutic cargo, the term “bioactive” can mean any molecule that has a therapeutic effect on living matter, including both treatment and prophylaxis. Living matter includes living beings, organs, tissues and cells. The terms “cargo” and “bioactive cargo” are used interchangeably throughout the specification and either a cargo or a bioactive cargo can be employed in any and all embodiments of the disclosure.

The term “therapeutic protein” shall be understood to relate to cargo that is specifically of protein or peptide origin and designed for the treatment and/or prophylaxis of a condition, disease and/or disorder. In addition to including large molecules and small molecules as hereinafter defined, therapeutic proteins may also include proteins and peptides that are not directly therapeutic per se but are endogenously active in the subject, such that they impart an indirect therapeutic effect. Endogenous activity is hereinafter defined.

The term “meganuclease” refers to an endonuclease having a double-stranded DNA target sequence of 12 to 45 bp. A meganuclease is either a dimeric enzyme, wherein each domain is on a monomer or a monomeric enzyme comprising the two domains on a single polypeptide.

The term “meganuclease domain” is intended the region which interacts with one half of the DNA target of a meganuclease and is able to associate with the other domain of the same meganuclease which interacts with the other half of the DNA target to form a functional meganuclease able to cleave said DNA target.

The term “single-chain meganuclease” is intended a meganuclease comprising two LAGLIDADG homing endonuclease domains linked by a peptidic spacer. The single-chain meganuclease is able to cleave a chimeric DNA target sequence comprising one different half of the two parent meganucleases target sequences. The single-chain meganuclease is also named single-chain derivative, single-chain meganuclease, single-chain meganuclease derivative or chimeric meganuclease.

The terms “large molecule”, “large molecule cargo”, “large molecule drug” or “biologic” and “biologics” are used interchangeably herein and shall be understood to relate to any large molecular agent (as can be defined by MW), nucleic acid, protein, polypeptide, or polysaccharide, which may be used for the treatment, prophylaxis and/or diagnosis of a condition, disease and/or disorder. For the purposes of this disclosure, a large molecule is typically larger than about 900 g/mol, for instance larger than about 1500 g/mol, larger than about 3000 g/mol, or occasionally even larger and possibly up to about 150.00 Da. Typically, the traditional route of administration to a subject for such large molecules is by injection.

The terms, “small molecule”, “small molecule cargo”, “small molecule drug” and “small molecule therapeutic” are used interchangeably herein and shall be understood to relate to any chemical or small molecular agent (as can be defined by MW), short peptide chain, monosaccharides, disaccharides, other small chain saccharides with a MW of less than about 900 Da, single domain antibodies (sdAbs) and antibody (Ab) fragments with a MW of less than about 900 Da, or amino acids, which may be used for the treatment, prophylaxis and/or diagnosis of a condition, disease and/or disorder. Small molecule agents are normally synthesized via chemical synthesis means, but may also be naturally derived, for instance via purification from natural sources, or may be obtained through any other suitable means or combination of techniques. A suitable definition of a “small molecule” may be any organic compound with a molecular weight of less than 900 g/mol (Dalton) that may help to regulate a biological process. The route of administration to a subject for such small molecules vary but typically include oral administration.

The terms “producer cell”, “cell source”, EV-producing cells” and “EV-producing cell source” are used interchangeably herein and shall be understood to relate to any cell from which the EVs of the present disclosure may be obtainable or derivable. Generally, EVs may be derived from essentially any cell source, be it a primary cell source or an immortalized cell line. The EV source cells may thus be any embryonic, fetal, or adult somatic stem cell types, including induced pluripotent stem cells (iPSCs) and other stem cells derived by any method, as well as any adult cell source. The source cells per the present disclosure may be selected from a wide range of cells and cell lines, for including but not limited to mesenchymal stem or stromal cells (obtainable from e.g., bone marrow, adipose tissue, Wharton's jelly, perinatal tissue, chorion, placenta, tooth buds, umbilical cord blood, skin tissue, etc.), fibroblasts, amnion cells and more specifically amnion epithelial (AE) cells optionally expressing various early markers, myeloid suppressor cells, M2 polarized macrophages, adipocytes, endothelial cells, fibroblasts, etc.

The term “endogenously loaded” and “endogenous loading” are used interchangeably herein and shall be understood to relate to any means of loading a desirable cargo, and/or fusion construct into an EV (e.g., exosome) by utilising existing internal mechanisms of a biological system, such as a producer cell, to produce EVs comprising the cargo and/or fusion protein of interest inside the cell. It will be appreciated that nucleic acid constructs might be exogenously loaded into a producer cell, but the resulting polypeptide derived from the engineered nucleic acid construct is made utilising the existing internal mechanisms of said biological system (e.g., cell) using material naturally available within the cell to generate the engineered fusion protein by natural means.

The term “exogenously loaded” and “exogenous loading” are used interchangeably herein and shall be understood to relate to any means of loading a desirable cargo and/or construct into an EV utilising a means that is external to the EV (e.g., exosome). Examples of exogenous loading include, but are not limited to, passive co-incubation, electroporation and transfection.

The terms “population of EVs”, and “EV population” are used interchangeably and shall be understood to encompass a homogenous set of individual EVs, wherein each EV in the population of EVs are the same. A population of EVs may therefore share any one or more of, for example, the same endosomal escape enhancer profile, the same cargo and/or the same EV protein. In other words, individual EVs, when present in a plurality and having the same shared characteristic or set of characteristics in common, constitute an EV population.

The terms “population of exosomes”, “exosomal population” and “exosome population” are used interchangeably and shall be understood to encompass a homogenous set of individual exosomes, wherein each exosome in the population of exosomes are the same. A population of exosomes may therefore share, for example, the same endosomal escape enhancer profile, the same cargo and/or the same exosome protein. In other words, individual exosomes, when present in a plurality and having a shared characteristic or set of characteristics in common, constitute an exosome population.

The term “subject” refers to any animal to which the EVs according to the present disclosure are administered. Typically, the subject is afflicted with or susceptible to be afflicted with a condition, disease and/or disorder, the treatment/prophylaxis or diagnosis of which would benefit from an EV according to the present disclosure. Preferably the subject is a mammal, more preferably human.

The terms “endogenously active” and “endogenous activity” are used interchangeably herein and shall be understood to relate to the therapeutic activity of the EVs and/or their cargo, specifically protein cargo, according to the present disclosure in a subject wherein the EVs cause the tissues and/or cells of a subject as hereinbefore described to generate their own means for the treatment or prophylaxis of a condition disease and/or disorder utilising the internal mechanisms of the tissues and/or cells of the subject to produce said means.

It will be appreciated that said means could be a drug cargo, large molecule or small molecule based therapeutic, or protein cargo as herein described.

Additionally, it should be noted that embodiments and features described in connection with one of the aspects and/or embodiments of the present disclosure also apply mutatis mutandis to all the other aspects and/or embodiments of the disclosure. For example, the endosomal escape enhancer, fragments and domains thereof and fusion proteins/polypeptides comprising said endosomal escape enhancer, described herein in connection with the EVs (e.g., exosomes) are to be understood to be disclosed, relevant and compatible with all other aspects, teachings and embodiments herein, for instance aspects and/or embodiments relating to the methods for producing or purifying the EVs or relating to the corresponding polynucleotide constructs described herein or the engineered EV-producing cells from which the EVs derive. Furthermore, certain embodiments described in connection with certain aspects, for instance the administration routes of the EVs comprising the therapeutic cargo molecule and optionally the fusion polypeptides, as described in relation to aspects pertaining to treating certain medical indications, may naturally also be relevant in connection with other aspects and/or embodiments, such as those pertaining to the pharmaceutical compositions comprising such EVs. Furthermore, all polypeptides and proteins identified herein can be freely combined in fusion proteins using conventional strategies for fusing polypeptides. Additional EV proteins, optionally combined with all other polypeptide domains, regions, sequences, peptides, groups herein, e.g., any multimerization domains, linker sequences, release domains, cargo molecules, endosomal escape domains protective moieties (e.g., albumin and/or ABD) and/or targeting moieties may also be included. Moreover, any and all features can be freely combined with any and all other features, e.g., any endosomal escape enhancer and/or cargo may be combined with any EV protein. Furthermore, when teachings herein refer to EVs in singular and/or to EVs as discrete natural nanoparticle-like vesicles, it shall be understood that all such teachings are equally relevant for and applicable to a plurality of EVs and populations of EVs. As a general remark, endosomal escape enhancers, the EV proteins, the additional domains and moieties, the cargo molecules, the targeting moieties and all other aspects, embodiments, and alternatives in accordance with the present disclosure may be freely combined in any and all possible combinations without deviating from the scope and the gist of the disclosure. Furthermore, any polypeptide or polynucleotide or any polypeptide or polynucleotide sequences (amino acid sequences or nucleotide sequences, respectively) of or relating to the present disclosure may deviate considerably from the original polypeptides, polynucleotides, and sequences, provided any given molecule retains the ability to carry out the desired technical effect associated therewith. Provided their biological properties are maintained, the polypeptide and/or polynucleotide sequences according to the present disclosure may deviate by typically as much as 50% and in some instances as much as 30% (calculated using, for instance, BLAST or ClustalW) as compared to the native sequence, although a sequence identity or similarity that is as high as possible is preferable (for instance at least 60%, at least 70%, at least 80%, or at least 90% or higher). Standard methods in the art may be used to determine sequence identity or homology. For example, PILEUP and BLAST algorithms can be used to calculate homology or line up sequences. The combination (fusion) of e.g., several polypeptides implies that certain segments of the respective polypeptides may be replaced and/or modified and/or that the sequences may be interrupted by insertion of other amino acid stretches, meaning that the deviation from the native sequence may be considerable provided the key properties (e.g., ability to extend half-life, ability to traffic a fusion construct to an EV, targeting capabilities, etc.) are conserved. Similar reasoning thus naturally applies to the polynucleotide sequences encoding such polypeptides. Any SEQ ID NOs mentioned herein in connection with peptides, polypeptides and proteins shall only be seen as examples and for information only, and all peptides, polypeptides and proteins shall be given their ordinary meaning as the skilled person would understand them. Thus, as above-mentioned, the skilled person will also understand that the present disclosure encompasses not merely the specific SEQ ID NOs and/or accession numbers referred to herein, but also variants and derivatives thereof. All proteins, polypeptides, peptides, nucleotides, and polynucleotides mentioned herein are to be construed according to their conventional meaning as understood by a skilled person, unless otherwise defined.

### EXAMPLES

The materials, methods, and examples described hereinbelow detail embodiments according to the present disclosure and support the understanding thereof. In addition, the materials, methods, and examples are illustrative only and are not intended to be limiting in any way.

Although alternative methods and materials similar or equivalent to those described herein can be used in the practice or testing of the present disclosure, suitable methods and materials are described below.

### Example 1: Protein Constructs

The inventors' aim was to develop novel engineered EVs with greater efficiency in delivering functional proteins into recipient cells both in vitro and in vivo. Constructs were prepared to prove the inventors' theory and the delivery of an exemplary cargo, such as Cre. The inventors postulated that two platforms could be useful in this regard. Said platforms were VSV-G Endosomal Domain (an EV protein, preferably exosomal protein) Intein Cargo (VEDIC) and VSV-G Foldon Intein Cargo (VFIC), respectively. The nomenclature derives from the preferred components used.

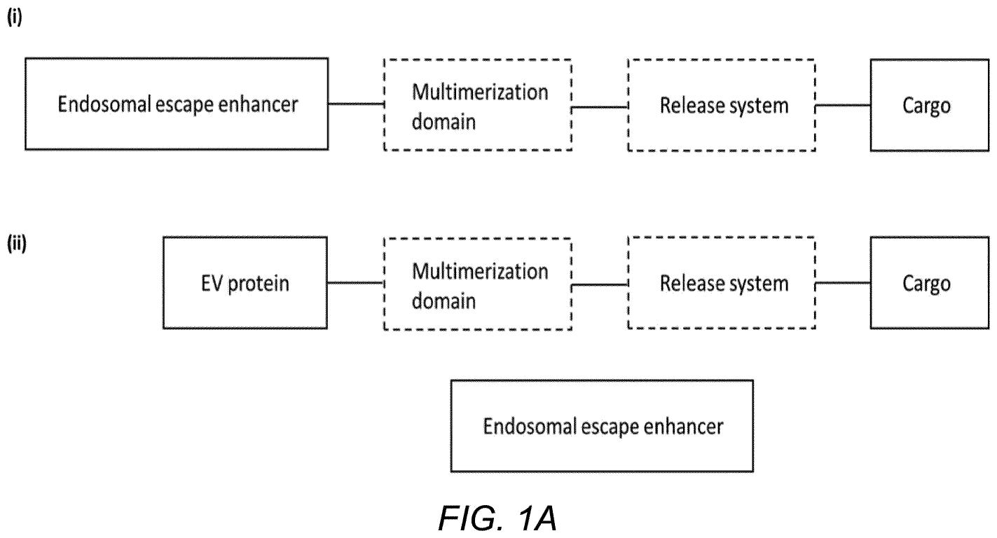

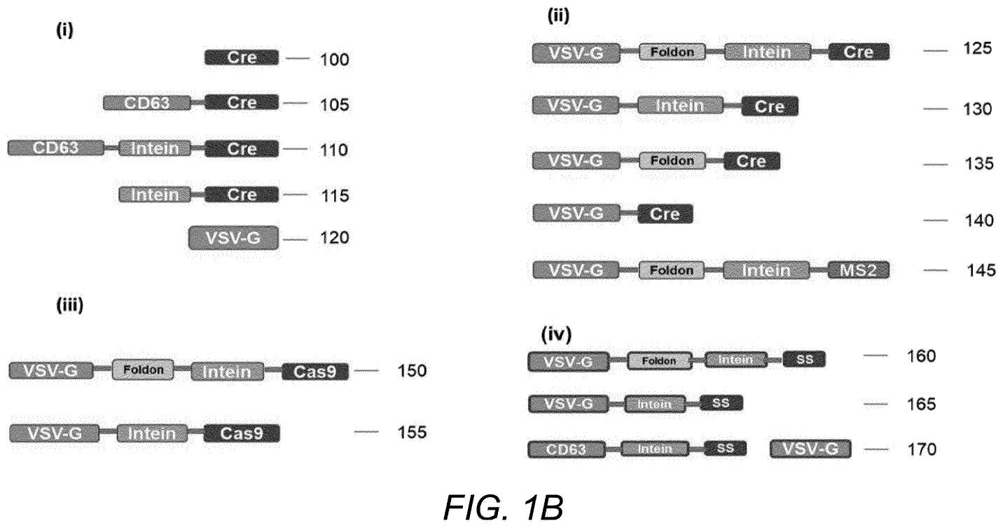

Referring to FIG. 1A there is provided an overview of the component parts (or domains) of the protein constructs and how they relate to one another. There are two alternatives. Optional features are shown by a dotted line.

Referring specifically to FIG. 1A (i) there is provided one protein construct having at least an endosomal escape enhancer and a cargo. Optionally there is provided a multimerization domain and/or a release system. Each of the multimerization domain and release system are, independently from one another, positioned between the endosomal escape enhancer and the cargo. When both the multimerization domain and release system are included, the multimerization domain is positioned between the endosomal escape enhancer and the release system, and the release system is positioned between the multimerization domain and the cargo.

Referring specifically to FIG. 1A (ii) there is provided two protein constructs. A first protein construct having at least an EV protein and a cargo and a second, separate, protein construct having the endosomal escape enhancer. Optionally, in the first construct, there is provided a multimerization domain and/or a release system. Each of the multimerization domain and release system are, independently from one another, positioned between the EV protein and the cargo. When both the multimerization domain and release system are included, the multimerization domain is positioned between the EV protein and the release system, and the release system is positioned between the multimerization domain and the cargo.

It will be appreciated that, while the above refers to the protein construct, the same structure and arrangement of domains may also equally apply to the corresponding polynucleotide construct.

Referring now to FIG. 1B there is provided an overview of several more specific protein constructs that were prepared, and which fall under the more general protein construct design outlined hereinbefore in FIG. 1A. FIG. 1B also shows the component parts of those constructs as follows:

Referring specifically to FIG. 1B(i) there is provided a platform comprising an endosomal escape enhancer, an EV protein, a release system and a cargo (referred to as VEDIC), the VEDIC platform comprising:
VSVG-EV protein-Intein-Cargo

The construct Cre 100 has a protein of interest (Pol), wherein the Pol is a cargo (e.g., Cre) but without a release system (e.g., intein) and without an EV protein (e.g., CD63). This construct may comprise either the DNA sequence SEQ ID No. 10 or amino acid sequence SEQ ID No. 22.

The construct CD63-Cre 105 has an EV protein (e.g., CD63) and a cargo (e.g., Cre) but without a release system (e.g., intein). This construct may comprise either the DNA sequences SEQ ID No. 4 and SEQ ID No. 10 or amino acid sequences SEQ ID No. 16 and SEQ ID No. 22.

The construct CD63-Intein-Cre 110 has an EV protein (e.g., CD63) and a cargo (Cre) and a release system (e.g., intein). This construct may comprise either the DNA sequences SEQ ID No. 4, SEQ ID No. 10 and SEQ ID No. 8 or amino acid sequences SEQ ID No. 16, SEQ ID No. 22 and SEQ ID No. 20.

The construct Intein-Cre 115 has a cargo (e.g., Cre) and release system (e.g., intein) but without an EV protein. This construct may comprise either the DNA sequences SEQ ID No. 10 and SEQ ID No. 8 or amino acid sequences SEQ ID No. 22 and SEQ ID No. 20.

The construct VSV-G: 120 is a separate construct comprising an endosomal escape enhancer only. This construct may comprise either the DNA sequence SEQ ID No. 1 or amino acid sequence SEQ ID No. 13.

The VEDIC platform requires multiple plasmids for transfection to produce EVs.

Referring specifically to FIG. 1B (ii) there is provided a platform comprising an endosomal escape enhancer, a multimerization domain, a release system and cargo (referred to as VFIC), the VFIC platform comprising:
VSVG-Foldon-Intein-Cargo

The construct VSV-G-Foldon-Intein-Cre 125 has an endosomal escape enhancer (e.g., VSV-G), a multimerization domain (e.g., foldon), a self-cleaving protein (intein) and a cargo (e.g., Cre). This construct may comprise either the DNA sequences SEQ ID No. 1, SEQ ID No. 9 and SEQ ID No. 8 or amino acid sequences SEQ ID No. 13, SEQ ID No. 21 and SEQ ID No. 20.

The construct VSV-G-Intein-Cre 130 has an endosomal escape enhancer (e.g., VSV-G), a self-cleaving protein (intein). and a cargo (e.g., Cre). This construct may comprise either the DNA sequences SEQ ID No. 1, SEQ ID No. 8 and SEQ ID No. 10 or amino acid sequences SEQ ID No. 13, SEQ ID No. 22 and SEQ ID No. 20.

The construct VSV-G-Foldon-Cre 135 has an endosomal escape enhancer (e.g., VSV-G), a multimerization domain (e.g., foldon) and a cargo (e.g., Cre).

This construct may comprise either the DNA sequences SEQ ID No. 1, SEQ ID No. 9 and SEQ ID No. 10 or amino acid sequences SEQ ID No. 13, SEQ ID No. 21 and SEQ ID No. 22.

The construct VSV-G-Cre 140 has an endosomal escape enhancer (e.g., VSV-G) and a cargo (e.g., Cre). This construct may comprise either the DNA sequences SEQ ID No. 1 and SEQ ID No. 10 or amino acid sequences SEQ ID No. 13 and SEQ ID No. 22.

The construct VSV-G-Foldon-Intein-MS2 145 has an endosomal escape enhancer (e.g., VSV-G) a multimerization domain (e.g., foldon) a self-cleaving protein (e.g., intein) and an aptamer capable of binding to certain RNA sequences (e.g., MS2). This construct may comprise either the DNA sequences SEQ ID No. 1, SEQ ID No. 9 and SEQ ID No. 8 or amino acid sequences SEQ ID No. 13, SEQ ID No. 21 and SEQ ID No. 20. Said construct serves as a negative control.

The VFIC platform provides the benefit of having all components in one plasmid meaning that only one plasmid is needed for the transfection to produce EVs. Another advantage is that the resulting EVs have an equivalent efficiency to the alternative VEDIC platform. It is to be appreciated that the VSV-G has a membrane anchoring domain that secures the VSV-G into the EV membrane (or exosomal membrane). Presence of a multimerization domain (e.g., Foldon) supports the stable formation (i.e., reduced degradation) of a multimer protein, which is capable of enhanced binding with other protein and/or ligand subunits in the multimer, as described in the present disclosure. As such, it makes it possible to have a stable fusion protein comprising VSV-G and a self-cleaving protein and/or cargo.

Referring specifically to FIG. 1B (iii) there is provided the VFIC platform 150 wherein the cargo is a CRISPR protein (e.g., Cas9). The construct VSVG-Intein-Cas9 155 comprises an endosomal escape enhancer (e.g., VSV-G), a release system (e.g., intein) and a cargo (e.g., Cas9) and serves as a control.

This construct may comprise either the DNA sequences SEQ ID No. 1, SEQ ID No. 8 and SEQ ID No. 12 or amino acid sequences SEQ ID No. 13, SEQ ID No. 20 and SEQ ID No. 24.

Referring specifically to FIG. 1B (iv) there is provided the VFIC platform 160 wherein the cargo is a super suppressor of NFkB (e.g., IKBα). While not shown, it is to be appreciated that preferably a mutated form of IKBα is employed, which does not easily disassociate from NFkB. The construct VSVG-Intein-SS 165 comprises an endosomal escape enhancer (e.g., VSV-G) a release system (e.g., intein) and a cargo (e.g., IkBα). The construct CD63-Intein-SS wherein the endosomal escape enhancer (VSV-G) forms part of a separate construct 170 comprises an EV protein (e.g., CD63), a release system (e.g., intein) and a cargo (e.g., IkBα). This construct may comprise either the DNA sequences SEQ ID No. 1, SEQ ID No. 8 and SEQ ID No. 11 or amino acid sequences SEQ ID No. 13, SEQ ID No. 20 and SEQ ID No. 23. The construct comprising CD63-Intein-SS without VSV-G serves as a control.

It will be appreciated that the above platforms, while exemplary and support the inventive concept, they are not exhaustive. As such, they should not be read to limit the scope of the disclosure in any way.

For example:
VSV-G could be swapped out for another endosomal escape enhancer such as CVG and PFV Envelope.
The EV protein can be selected from any one of a non-limiting membrane-associated EV protein (e.g., CD2, CD138, CD40L, DLL4, JAG1, MFGE8, FLOT1, FLOT2 and SLIT2), a single-pass transmembrane EV protein (e.g., PTGFRN, Lamp2B), a multi-pass transmembrane EV protein (e.g., CD47, CD133, CD151, CD184) and more preferably a tetraspanin (e.g., CD63, CD9, and CD81).
The intein may be full length wild-type, or any derivative, domain, variant, mutant, or regions thereof provided that the desired properties (e.g., self-cleaving) are conserved, such as delta intein and delta-intein-CM. The intein may even be another release mechanism other than intein provided the same desired effect (e.g., release of a functional [unconjugated] cargo) is achieved.
The foldon may be swapped for another multimerization domain provided the same effect is conserved.
The cargo may be Cre, CRISPR associated proteins (e.g., Cas9) and/or RNA binding proteins (RBPs) capable of trafficking RNAs (e.g., gRNA) into an EV or exosome.

### Constructs Generation

All the transgenes (VSV-G, VSVG-Foldon-Intein-Cre, VSVG-Foldon-Intein-M1 PCSK9, VSVG-Foldon-Intein-SS (super suppressor), CD63-Intein-Cre, CD81-Intein-Cre, CD9-Intein-Cre, PTGFRN-Intein-Cre, CD63-Cre, Intein-Cre, Cre, PFV-Intein-Cre, CVG-Intein-Cre and VSVG K47Q) were obtained from IDT (Integrated DNA Technologies, USA). The transgenes were first cloned into pLEX vector by using EcoRI and XhoI sites. After cutting with Kpn21 and performing self-ligation, VSVG-Intein-Cre was obtained from VSVG-Foldon-Intein-Cre. Similarly, after cutting with MluI, VSVG-Foldon-Cre was obtained. To get VSVG-Cre, MluI was used to cut VSVG-Intein-Cre and then self-ligation was performed. The construct CD63-Intein-SS was obtained by cutting VSVG-Foldon-Intein-SS and CD63-Intein-Cre with BamHI and XhoI, and then inserted SS to CD63-Intein.

### Example 2: EV Preparation for Analysis of Delivery

HEK-293T cells were used to produce the functional EVs.

### Cell Culture

The HEK-293T cells were kept in DMEM medium (high glucose) supplemented with 10% fetal bovine serum (FBS, Gibco, USA) and 1% Anti-anti (Gibco, USA). Cells were cultured at 37° C. in a humidified air atmosphere containing 5% CO2. The reporter cell lines (Hela-TL, T47D-TL and B16F10-TL) were cultured with the same medium and the same conditions as HEK-293T cells.

### EV Production

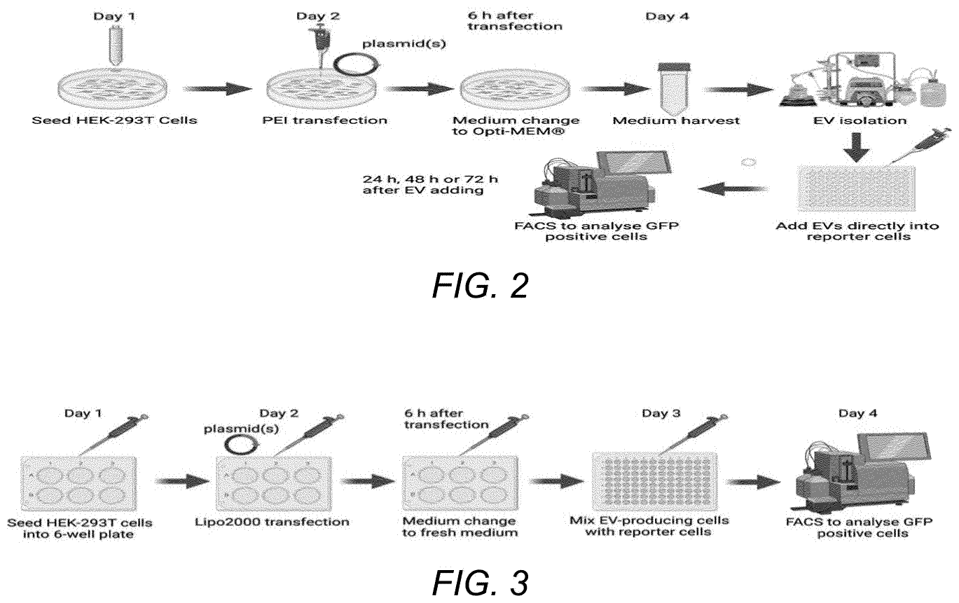

The EVs were produced by transient transfection of the transgenes by using polyethylenimine (PEI). As illustrated in FIG. 2 cells were seeded out then cultured for one day employing conventional methods. For example, the HEK-293T cells were seeded into 15-cm dishes with 10 million cells/dish with full DMEM medium. The day after, the cells were transfected by the transgenes and the medium was changed to Opti-MEM medium (Gibco, USA) with 1% Anti-anti 6 h after transfection. After 48 h of transfection, the conditional medium (CM) was collected and went through centrifugation (700 g, 5 min followed by 2000 g, 10 min). The supernatant was then filtered by 0.22 μm filter system.

It will be appreciated that the conventional methods used are well known and other producer cells/cell sources may equally be employed. Inventors envisage that said methods can be routinely adapted to any suitable producer cell type used by routine modification of, for example, the media composition and the conditions under which the cells are grown.

On day two. FIG. 2 shows the cells were transfected with a plasmid comprising either VEDIC or VFIC or alternatively another construct within the scope of the present disclosure.

Six hours post transfection, as depicted in FIG. 2, the medium was changed to opti-MEM® or opti-MEM® without FBS. Opti-MEM® without FBS is a stressor to the cells, causing enhancement of EV (e.g., exosome) production. OptiMEM® is used for easier purification and/or isolation of the EVs after harvest. At day four the cells were suspended in a harvest medium from which the EVs were isolated as shown in FIG. 2.

### EV Isolation

EV isolation was performed by conventional means such as that described below.

Tangential flow filtration (TFF, MicroKross, 20 cm2, Spectrum labs) was used to isolate EVs from the filtered CM. The cutoff of TFF was 300 kDa and particles bigger than 300 kDa remained in the system and were concentrated. And then the concentrated particles were further concentrated by Amicon Ultra-15 100 kDa (Millipore) spin filter, centrifuged at 4000×g for 30 min to several hours at 4° C. depending on the amount of EVs in the samples. Finally, the concentrated EVs were collected in maxirecovery 1.5 ml Eppendorf tubes (Axygene, USA) and the concentrations were detected/confirmed by Nanoparticle Tracking Analysis (NTA).

### Nanoparticle Tracking Analysis (NTA)

The EV samples were diluted with freshly prepared 0.22 μm-filtered PBS before checking the particle sizes and concentrations of the samples with a NanoSight NS500 instrument. 5 videos with time longer than 30 seconds were taken at the cameral level of 15 in light scatter mode. The obtained data were analysed by the equipped NTA 2.3 software, and all the samples were analysed with the same constant setting.

Traffic Light reporter cells-Hela, T47D and B16F10-were employed for analysis of functional delivery of the desired protein (e.g., Cre). Upon the right impetus, such as cre, recombination occurred causing GFP to be expressed. The GFP expression caused a visual change in the reporter cells from red to green.

The isolated EVs were added directly to said reporter cells by transient transfection. For less sensitive reporter cells (e.g., Hela-TL cells) EVs were added at 1E10, 1E9 and 1E8 doses. For more sensitive reporter cells (e.g., T47D-TL and B16F10-TL cells) EVs were added at 1E9, 1E8 and 1E7 doses. B16F10-TL cells were found to be the most sensitive.9

FACS analysis was performed to detect GFP positive cells at 24 hr, 48 hr and 72 hr timepoints after adding the EVs to the reporter cells.

### EV Uptake in Reporter Cells

The reporter cells were seeded into 96-well plates one day before adding the engineered EVs. The EVs were removed from the wells by discarding the conditional medium at different time points (5 min, 10 min, 15 min, 20 min, 30 min, 60 min, 2 h, 4 h, 6 h, 8 h, 12 h, 16 h, 24 h, 36 h and 48 h) and then washed once by PBS before adding fresh medium. Finally, the percentage of GFP positive cells was measured by MACSQuant.

### MACSQuant Flow Cytometry

After the different traffic-light reporter cells were added into EVs at different time points, they were checked for GFP expression by MACSQuant flow cytometry (Miltenyi biotec, Germany).

In brief, the cells in 96-well plates were washed by PBS once after discarding supernatant and then the cells were trypsinized for 5 min at 37° C. The cells were then resuspended in cell medium supplemented with 10% FBS. After adding DAPI to check the cell viability, the cells were sampled by MACSQuant with the same setting for all the measurements of one specific reporter cell line. Finally, the data were analysed using Flowjo™ software to calculate the percentage of GFP positive cells.

### Example 3: Co-Culture System for Analysis of Delivery

The inventors found existing systems to lack sensitivity to effectively study the functional delivery of proteins into recipient cells. A co-culture system was thus developed and was found to be very sensitive to check the functional delivery.

As illustrated in FIG. 3 cells were seeded out then cultured for one day employing conventional methods as described in Example 2 above.

Particularly, HEK-293T cells were seeded into a 6-well plate with 0.5 million cells per well.

On day two, as shown in FIG. 3, the cells were lipo-transfected with a plasmid comprising either VEDIC or VFIC or another construct in accordance with the present disclosure.

Particularly, when the cells reached 60-70% confluence, corresponding constructs were transfected into the wells by using lipofectamine2000 (Invitrogen, USA) according to the protocols provided by the manufacturer.

FIG. 3 shows that after six hours post transfection the medium was exchanged for fresh full medium.

In more detail at 6 h post transfection, the medium was changed to fresh medium (DMEM+10% FBS+1% Anti-anti) to reduce the toxicity of lipofectamine2000 used. At 24 h post plasmid transfection, the cells were trypsinized and counted and mixed with the corresponding reporter cells with the ratios 1:1 or 1:5 (ratio=EV-producing cells: reporter cells) in a 96-well plate.

Traffic Light reporter cells—Hela, T47D and B16F10—were employed for analysis of functional delivery of the desired protein. Reporter cells functioned as described in Example 2.

The EV-producing cells (e.g., HEK293T) were mixed with reporter cells and co-cultured for a day as is seen in FIG. 3. The EV-producing cells and reporter cells were mixed at a ratio of from about 1:1 to about 1:5, wherein as the ratio changed, there were more reporter cells present compared to EV producing cells (e.g., a ratio of 1 EV producing cell to 5 reporter cells).

After co-culturing for 24 h, the cells were trypsinized and measured by MACSQuant to check the percentage of GFP positive cells as described in Example 1 above.

FACS analysis was performed at day four to analyse GFP positive cells at 24 hr, 48 hr and 72 hr timepoints after EV-producing cells were mixed and co-cultured with reporter cells.

The lack of a sensitive system to study the functional delivery of proteins into recipient cells poses a challenge.

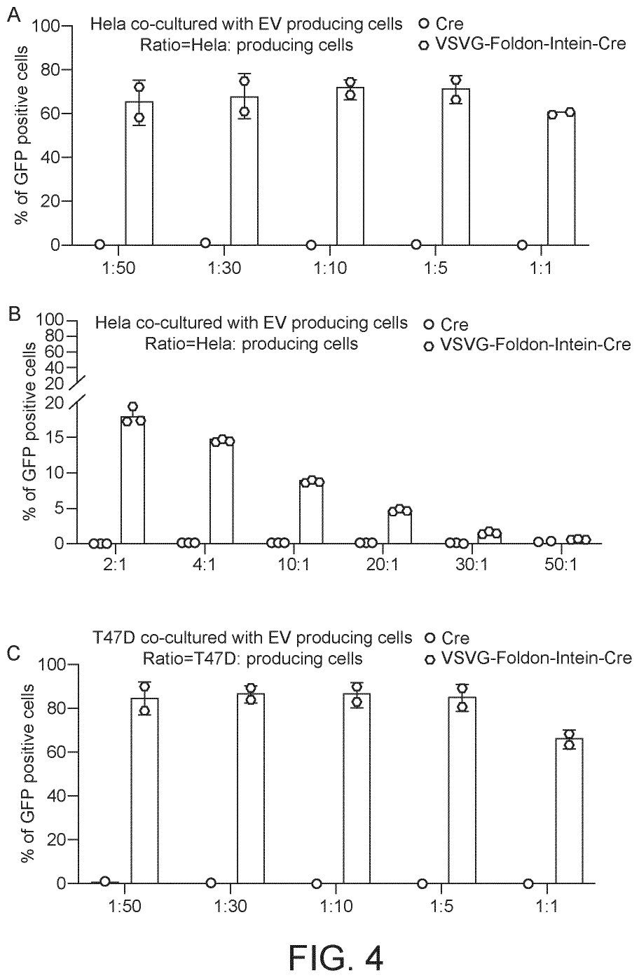

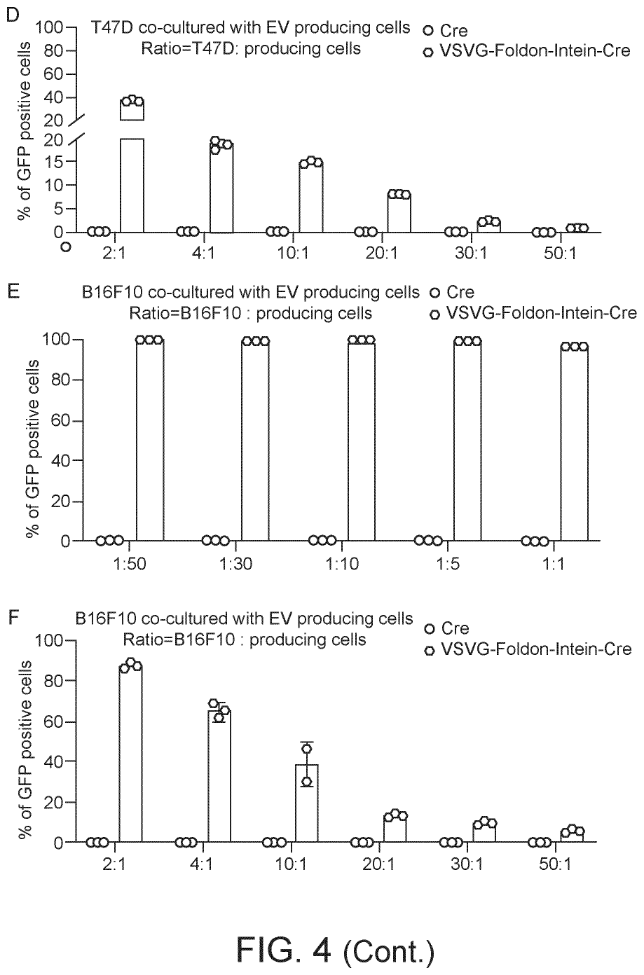

Referring to FIG. 4 the percentage (%) GFP positive cells (Y-axis) against cell-to-cell ratio dependent functional communication between EV producing cells and reporter cells (X-axis) is shown. GFP readout depends on the presence of freely available Cre in the cytoplasm of a cell. On the left-hand column (i.e., A, C and E) delivery saturation is shown. On the right-hand column (i.e., B, D and F) detection limitation is shown.

Referring specifically to FIG. 4A which shows the delivery saturation of EVs comprising either Cre only (blue circle; left-hand side) or VSVG-foldon-intein-cre (red circle; right-hand side). Said EVs were produced by the EV-producing cells. In this assay the reporter cells used were Hela cells.

Delivery saturation was shown to be between 1:5 and 1:1 in Hela cells.

Referring specifically to FIG. 4B, which shows detection limitation of the system wherein the system comprises EVs having Cre only (blue circle; left-hand side) or VSVG-foldon-intein-cre (red circle; right-hand side). In this assay the reporter cells used were Hela cells.

Referring specifically to FIG. 4C which shows the delivery saturation of EVs comprising either Cre only (blue circle; left-hand side) or VSVG-foldon-intein-cre (red circle; right-hand side). Said EVs were produced by the EV-producing cells. In this assay the reporter cells used were T47D cells.

Delivery saturation was shown to be between 1:5 and 1:1 in T47D cells.

Referring specifically to FIG. 4D, which shows detection limitation of the system wherein the system comprises EVs having Cre only (blue circle; left-hand side) or VSVG-foldon-intein-cre (red circle; right-hand side). In this assay the reporter cells used were T47D cells.

Referring specifically to FIG. 4E which shows the delivery saturation of EVs comprising either Cre only (blue circle; left-hand side) or VSVG-foldon-intein-cre (red circle; right-hand side). Said EVs were produced by the EV-producing cells. In this assay the reporter cells used were B16F10 cells.

Delivery saturation was shown to be about 1:1 in B16F10 cells.

Referring specifically to FIG. 4F, which shows detection limitation of the system wherein the system comprises EVs having Cre only (blue circle; left-hand side) or VSVG-foldon-intein-cre (red circle; right-hand side). In this assay the reporter cells used were B16F10 cells.

Delivery saturation was between 1:5 and 1:1 for Hela, T47D and B16F10 cells. B16F10 cells, which are a newly created reporter cell system, were confirmed to be comparable to commercially available reporter cells (e.g., Hela and T47D). Even where the system comprised 50 times of the reporter cells versus the EV-producing cells, GFP could still be detected by FACS. As shown by comparison of FIGS. 4B, 4D and 4F detection sensitivity of a co-culture system comprising B16F10 reporter cells, as created by the inventors, outperformed the detection sensitivity of other commercially available reporter cells (e.g., Hela and T47D), as evidenced by the elevated % GFP positive cells in B16F10 cells at a ratio of 50:1 (reporter cell: EV-producing cell).

The Inventors have surprisingly shown a particularly sensitive co-culture system for use in checking the functional delivery when B16F10 reporter cells are used.

### Example 4: Confirming Functional Delivery of Cre by EVs Having Two Constructs

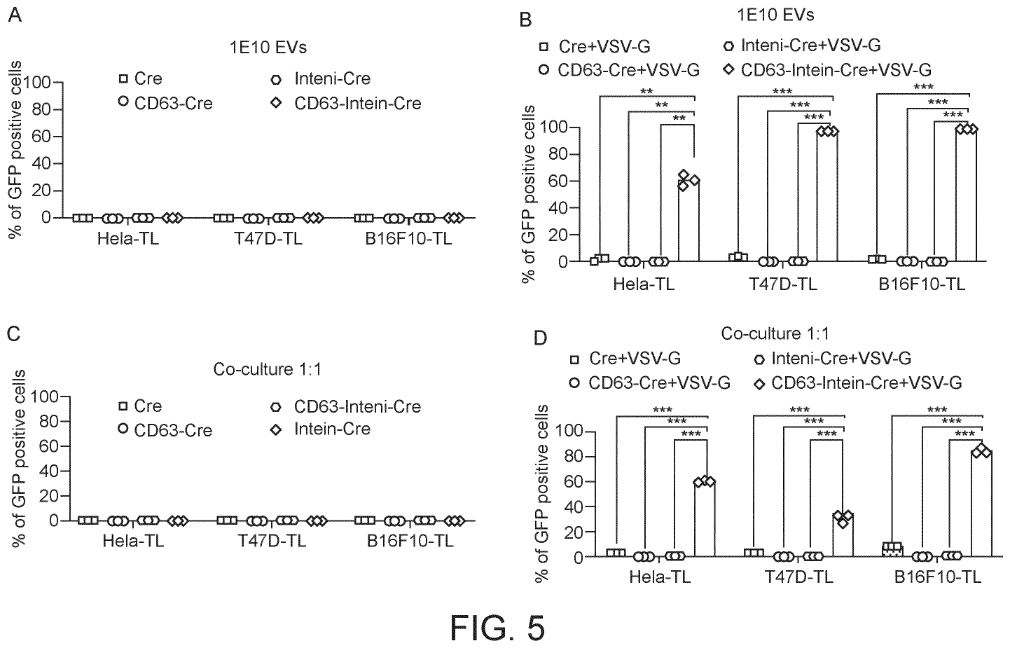

Referring to FIG. 5 the percentage (%) GFP positive cells (Y-axis) for various EVs comprising the protein constructs of interest in each of the reporter cells Hela-TL, T47D-TL and B16F10-TL (X-axis) is shown. On the left-hand column is shown constructs without VSV-G. On the right-hand column is shown constructs plus VSV-G as a separate construct. On the top row is shown results from the EV transfection assay. On the bottom row there is shown results from the co-culture assay.

It will be appreciated that FIG. 5 demonstrates double stable EVs and/or double stable cell lines, as two separate constructs are shown. One construct with cargo (and optionally an EV protein and/or self-cleaving protein) and one construct with an endosomal escape enhancer.

FIG. 5A shows the lack of delivery of functional Cre from EVs engineered to comprise the following constructs: Cre only (black circle; far left-hand side); CD63-Cre (blue circle; mid left-hand side); intein-Cre (red circle; mid right-hand side) and CD63-intein-cre (pink circle; far right-hand side) at an EV concentration of 1E10. Despite the expectation that inclusion of an intein and

EV protein would result in release of a functional cargo from/into and EV (or exosome) lack of GFP positive cells indicated that no functional Cre was observed. The inventor's postulate that this was due to the EV becoming trapped in the endo-lysosomal system.

FIG. 5B shows the lack of delivery of functional Cre from EVs engineered to comprise the following constructs: Cre+VSVG (black circle; far left-hand side); CD63-Cre+VSVG (blue circle; mid left-hand side); intein-Cre+VSVG (red circle; mid right-hand side). Also shown is delivery of functional Cre from EVs engineered to comprise the protein constructs CD63-intein-cre+VSVG (pink circle; far right-hand side) at an EV concentration of 1E10. Despite the expectation that two constructs would not result in an effective EV for reasons hereinbefore discussed, the inventors were pleasantly surprised to find that a double-stable cell and/or EV was in fact achieved, as demonstrated by GFP positive cells across several cell models and thus presence of functional Cre.

FIG. 5C shows the lack of delivery of functional Cre from EVs engineered to comprise the following constructs: Cre only (black circle; far left-hand side); CD63-Cre (blue circle; mid left-hand side); intein-Cre (red circle; mid right-hand side) and CD63-intein-cre (pink circle; far right-hand side) when EV producing cells and reporter cells were co-cultured 1:1.

FIG. 5D shows the lack of delivery of functional Cre from Evs engineered to comprise the following constructs: Cre+VSVG (black circle; far left-hand side); CD63-Cre+VSVG (blue circle; mid left-hand side); intein-Cre+VSVG (red circle; mid right-hand side). Also shown is delivery of functional Cre from Evs engineered to comprise the protein constructs CD63-intein-cre+VSVG (pink circle; far right-hand side) when EV producing cells and reporter cells were co-cultured 1:1.

The inventors surprisingly found that an EV sorting domain (a.k.a. EV protein, preferably exosomal protein), a release system, particularly self-cleaving protein and endosomal escape enhancer may be important for functional delivery of Cre protein.

The inventors have also surprisingly found that the combination of the EV protein, release system, particularly a self-cleaving protein and endosomal escape enhancer may be important for the functional delivery of the Cre protein regardless of whether EVs are added directly (immediate delivery) to the reporter cells or whether EV-producing cells are co-cultured with (sustained or prolonged effect) the reporter cells.

Functional delivery of Cre from Evs and from EV-producing cells was confirmed.

While the component parts of the engineered EVs might be known and could be employed, for example EV proteins for trafficking a Pol to the EV membrane, intein for release of the cargo from the EV and endosomal escape enhancers, there is no guarantee that all these features would work as expected in combination. Not least because engineering of protein constructs (particularly as fusion proteins) can negatively affect the desired activity of the proteins contained therein, for example:
the EV protein may no longer load/traffic the Pol to the EV membrane; and/or,
the intein no longer releases the cargo in a timely manner such that the cargo is loaded into the EV; and/or,
the intein no longer releases the cargo in a substantially free form, such that the cargo is still capable of carrying out the desired function/activity; and/or,
the endosomal escape enhancer is no longer able to free the EV from the endolysosomal system and/or enable delivery of the cargo into the cytoplasm of the cell.

As such, the inventors have surprisingly found that functional Cre is still delivered despite the abovementioned and similar possible issues arising due to engineering.

Other issues in EV engineering include achieving a double-stable cell and/or EV comprising two separate constructs contained therein for reasons hereinbefore discussed. As demonstrated by FIG. 5, the inventors have most surprisingly found that a double stable cell and/or EV is actually achieved. The inventor's postulate that this unusual double-stable characteristic is due to the release of the cargo into the lumen of the EV while displaying the endosomal escape enhancer on the surface of the EV (or exosome).

Furthermore, the inventors have postulated that the double stability results from reduced steric hinderance of the cargo with the endosomal escape enhancer.

### Example 5: Diversity of EVs Having Two Constructs Useful in the Delivery of Cre

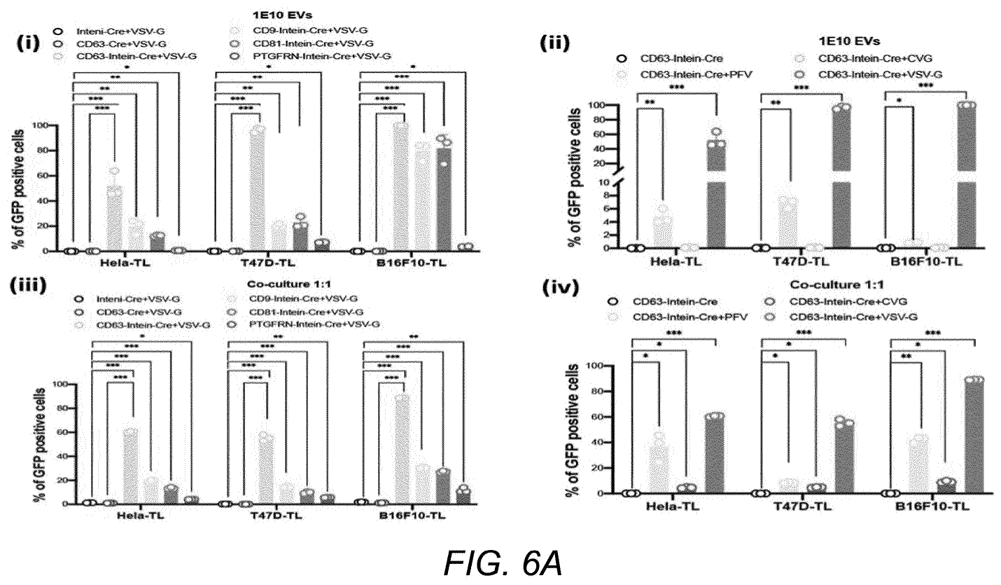

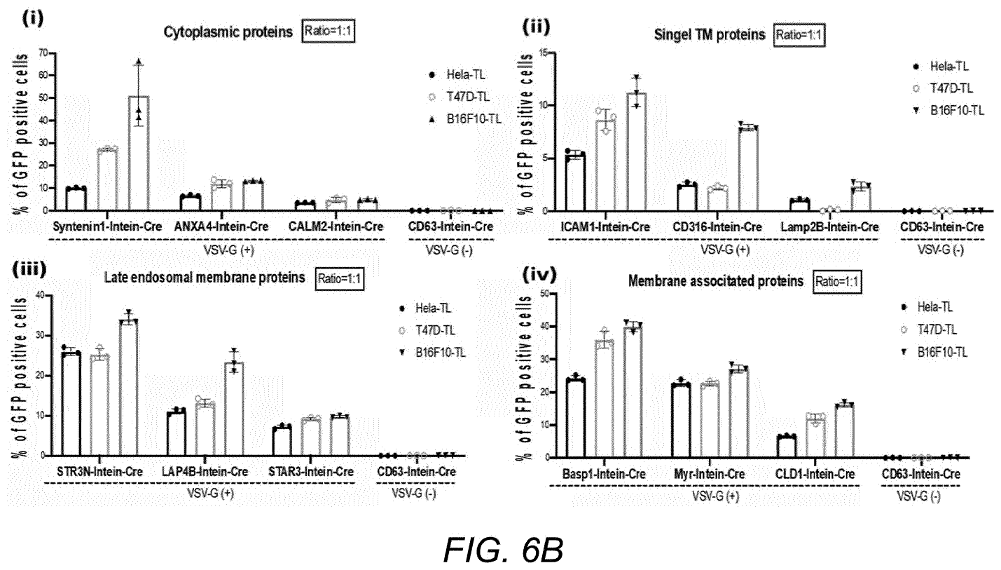

Referring to FIG. 6A there is shown the percentage (%) GFP positive cells (Y-axis) for various EVs comprising the protein constructs of interest in each of the reporter cells Hela-TL, T47D-TL and B16F10-TL (X-axis). On the left-hand set of graphs varying EV proteins were used. On the right-hand set of graphs is shown combinations of constructs wherein the endosomal escape enhancer was varied. On the top row is shown results from the EV transfection assay. On the bottom row there is shown results from the co-culture assay.

It will be appreciated that FIG. 6A demonstrates that the EV protein and/or the endosomal escape enhancer may be varied while still achieving functional delivery of cargo (such as Cre). It is postulated that the release system, preferably self-cleaving protein, may also be varied provided that key desired capabilities are retained (for example, desirable cleavage rate and/or release of the cargo in a substantially free form such that the cargo is functional/active); however, this is not shown in FIG. 6A.

Referring specifically to FIG. 6A(i) shows the percentage (%) GFP positive cells (Y-axis) for the following constructs: intein-Cre+VSVG (black circle; far left-hand side); CD63-Cre+VSVG (blue circle; mid left-hand side); CD63-intein-Cre+VSVG (green circle; inner left); CD9-intein-cre+VSVG (light blue circle; inner right); CD81-intein-cre+VSVG (purple circle; mid right); and PTGFRN-intein-Cre+VSVG (red circle; far right) at an EV concentration of 1E10.

Referring specifically to FIG. 6A (ii) shows the percentage (%) GFP positive cells (Y-axis) for the following constructs: CD63-intein-Cre+VSVG (black circle; far left-hand side); CD63-intein-Cre+PFV (light blue circle; mid left-hand side); CD63-intein-Cre+CVG (green circle; mid right); CD9-intein-cre+VSVG (purple circle; far right) at an EV concentration of 1E10.

Referring specifically to FIG. 6A (iii) shows the percentage (%) GFP positive cells (Y-axis) for the following constructs: intein-Cre+VSVG (black circle; far left-hand side); CD63-Cre+VSVG (blue circle; mid left-hand side); CD63-intein-Cre+VSVG (green circle; inner left); CD9-intein-cre+VSVG (light blue circle; inner right); CD81-intein-cre+VSVG (purple circle; mid right); and PTGFRN-intein-Cre+VSVG (red circle; far right) when EV producing cells and reporter cells were co-cultured 1:1.

Referring specifically to FIG. 6A (iv) shows the percentage (%) GFP positive cells (Y-axis) for the following constructs: CD63-intein-Cre (black circle; far left-hand side); CD63-intein-Cre+PFV (blue circle; mid left-hand side); CD63-intein-Cre+CVG (red circle; mid right); CD63-intein-cre+VSVG (purple circle; far right) when EV producing cells and reporter cells were co-cultured 1:1.

The inventors have found that other EV sorting domains and endosomal escape enhancers also worked equally well to deliver functional Cre. The inventors confirmed the diversity of the system in that other combinations of EV proteins (or exosomal proteins) and endosomal escape enhancers will also function as found by the inventors, at least when the release system is maintained unchanged (e.g., intein). For similar reasons discussed under Example 4 the inventors have surprisingly found that double-stable cells and/or EVs comprising various constructs comprising one construct having an EV protein-intein-cargo+an endosomal escape enhancer (as a separate construct).

Referring now to FIG. 6B, which shows the percentage (%) GFP positive cells (Y-axis) for various EVs comprising the protein constructs of interest in each of the reporter cells Hela-TL (^{•}), T47D-TL (^{∘}) and B16F10-TL (^{▴}) (X-axis) in the presence of the second construct having VSV-G (+) and when EV producing cells and reporter cells are co-cultured 1:1. The construct CD63-intein-cre minus (−) VSV-G construct (far right) was used as control.

The inventors have found that other types of EV protein were also explored under co-culture.

Referring specifically to FIG. 6B (i), which shows the percentage (%) GFP positive cells (Y-axis) for the following constructs from left to right: syntenin 1-intein-Cre (far left-hand side); ANXA4-intein-Cre (mid left-hand side); CALM2-intein-Cre (mid right).

Cytoplasmic proteins, which are luminal proteins, such as syntenin 1, ANXA 4 and CALM2 were also found to suitably work.

Referring now to FIG. 6B (ii), which shows the percentage (%) GFP positive cells (Y-axis) for the following constructs from left to right: ICAM 1-intein-Cre (far left-hand side); CD316-intein-Cre (mid left-hand side); Lamp2B-intein-Cre (mid right).

Single transmembrane proteins, which have one TM domain, such as ICAM1, CD316 and Lamp2B were also found to suitably work.

Referring now to FIG. 6B (iii), which shows the percentage (%) GFP positive cells (Y-axis) for the following constructs from left to right: ICAM 1-intein-Cre (far left-hand side); CD316-intein-Cre (mid left-hand side); Lamp2B-intein-Cre (mid right).

Late endosomal membrane proteins are a group of multi-pass membrane proteins that determine the fate of cargos inside late endosomes, either for forming ILVs or for lysosomal degradation. Examples of late endosomal membrane proteins include, but are not limited to, STR3N, LAP4B and STAR3 and were also found to suitably work.

Referring now to FIG. 6B (iv), which shows the percentage (%) GFP positive cells (Y-axis) for the following constructs from left to right: Basp 1-intein-Cre (far left-hand side); Myr-intein-Cre (mid left-hand side); CLD1-intein-Cre (mid right).

Membrane associated proteins bind to the inner surface (or inner leaflet) of the EV membrane (or exosomal membrane) and serve as EV-Sorting domains.

Examples of membrane associated proteins include, but are not limited to, Basp1, Myr and CLD1 and were also found to suitably work.

As hereinbefore discussed, it is postulated that the release system, preferably self-cleaving protein, may also be varied provided that key desired capabilities are retained (for example, desirable cleavage rate and/or release of the cargo in a substantially free form such that the cargo is functional/active); however, this is not shown in FIG. 6.

### Example 6: Confirmation of Endosomal Escape

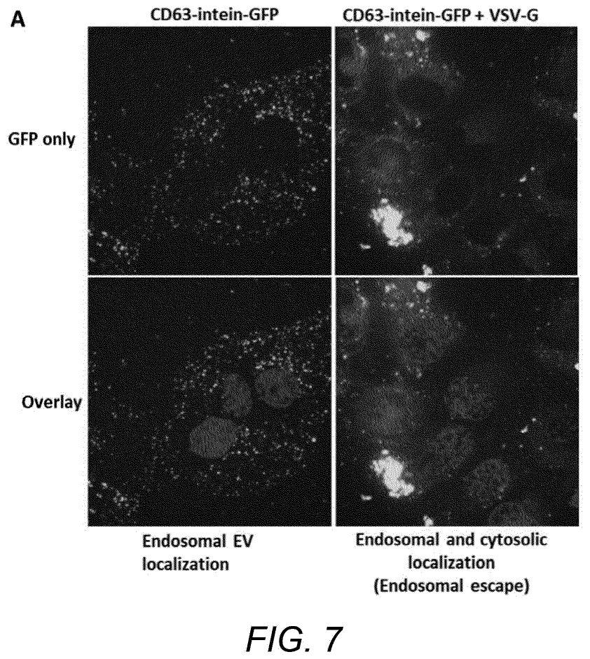

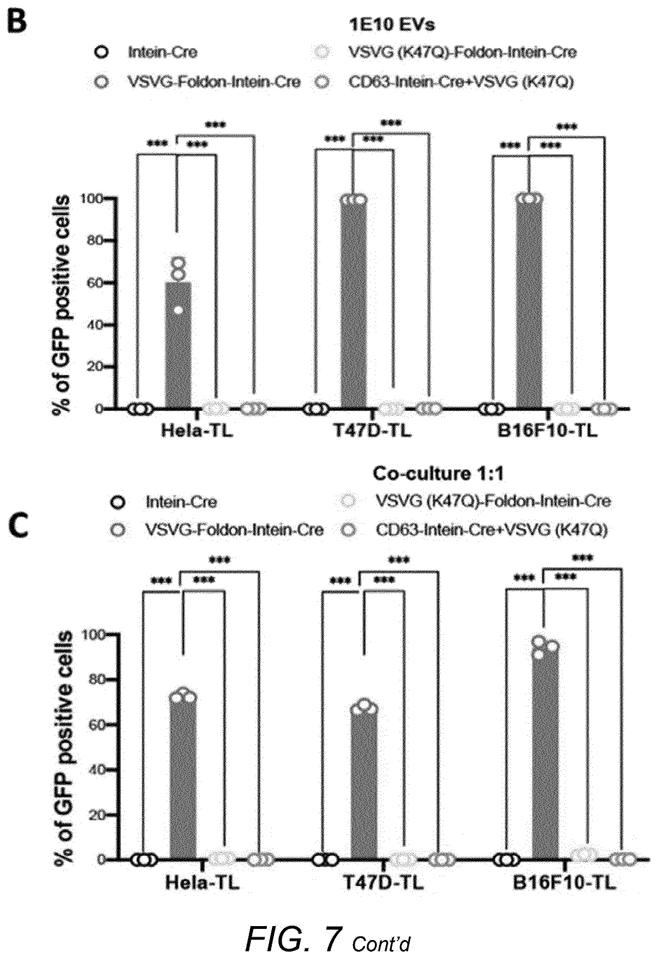

Referring to FIG. 7 an endosomal escape enhancer was shown to enable endosomal escape to deliver cargo to the cytoplasm.

Referring specifically to FIG. 7A, which shows a set of confocal microscopy images. The top left quadrant (quadrant 1) shows GFP (green) staining of CD63-intein present in the cytosol. The top right quadrant (quadrant 2) shows GFP (green) staining of CD63-intein when combined with VSVG (as a separate construct) present in the cytosol. The bottom left quadrant (quadrant 3) shows an overlay of the endosomal EV localization (blue). The bottom right quadrant (quadrant 4) shows an overlay of the endosomal EV localization (blue) when combined with VSVG (as a separate construct). The bright (green) patch in the bottom left corner of quadrants 2 and 4 show that CD63-intein-GFP escapes the endo-lysosomal system and is delivered to the cytosol of the cell when VSVG (an example of an endo-lysosomal escape enhancer) is also present.

The inventors found that only in the presence of VSV-G was an obvious cytosolic GFP observed, indicating the importance of an endosomal enhancer such as VSV-G. It is to be appreciated that two separate constructs (e.g., one comprising CD63 and intein; and one comprising VSVG) would not readily be anticipated by the field to function effectively together in an EV for reasons as hereinbefore described. The inventors have surprisingly found that a double-stable cell and/or EV is achieved, perhaps in part due to the addition of the intein meaning that with the cargo being released into the EV, steric hindrance is avoided with the portion of VSVG displayed on the outside of the EV as is important for endosomal escape. In this arrangement that enables a double-stable cell and/or EV to practically be achieved, the dual effect of endosomal escape and release of the cargo in a functional/active form into the cytoplasm of a cell becomes reality (as opposed to a theoretical concept). This effect of the double-stable construct is confirmed by knockout of VSVG action by use of the K47Q mutant (see FIG. 7B (pink circle; far right)).

Referring specifically to FIG. 7B which shows the percentage (%) GFP positive cells across report cell types (Hela-TL, T47D-TL and B16F10-TL) of the following constructs: intein-Cre (black circle; far left); VSVG-foldon-intein-Cre (red circle; mid left); VSVG (K47Q)-Foldon-Intein-Cre (light blue circle; mid right) and CD63-intein-Cre+VSVG (K47Q) (pink circle; far right).

The mutant form of VSV-G (K47Q) was used to demonstrate that the observed effect was indeed reliant on functional VSVG. The ability to bind to the receptors on the surface of cells, was lost and the construct comprising said K47Q thus did not deliver Cre proteins, indicating that receptor mediated endocytosis is important for the delivery of cargos by EVs. This effect was observed in both the double-stable construct as well as the single construct (VSVG-foldon-intein-cre).

While certain endosomal escape enhancers are exemplified, the inventors anticipate any viral fusogenic protein can most likely work similarly to those tested. This includes the heVLPs like the human endogenous retroviral envelope proteins synctyin 1 and synctyin 2.

### Example 7: Confirmation of Presence and Location of Co-Localisation

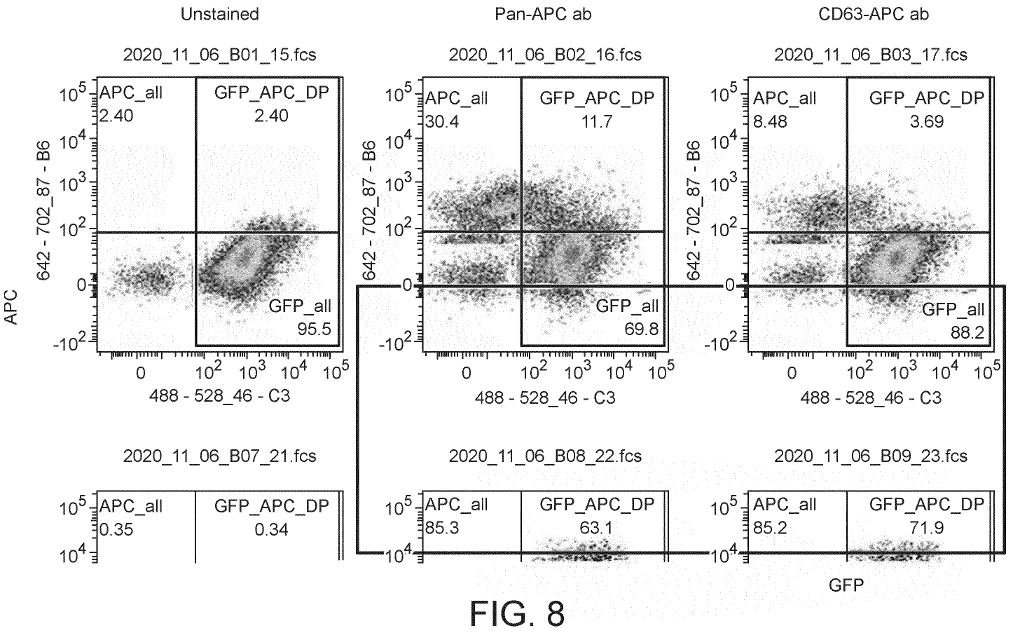

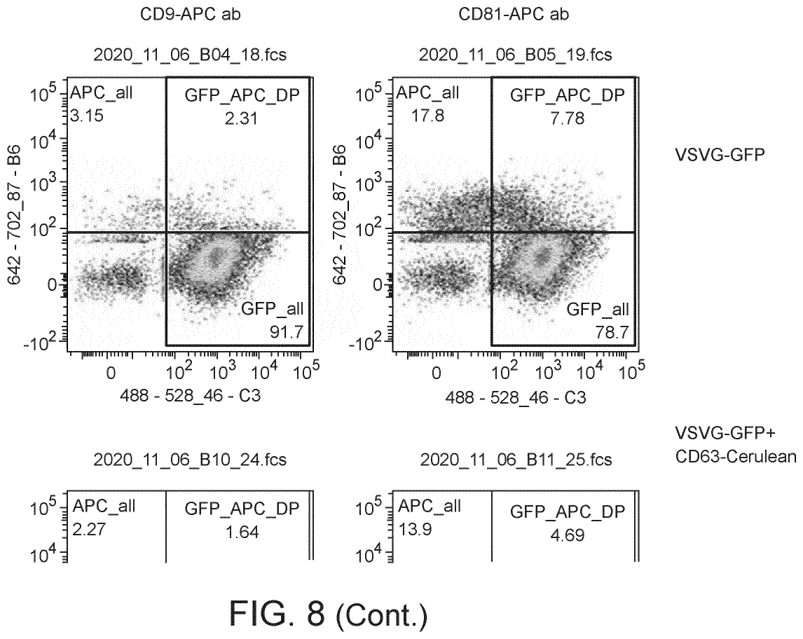

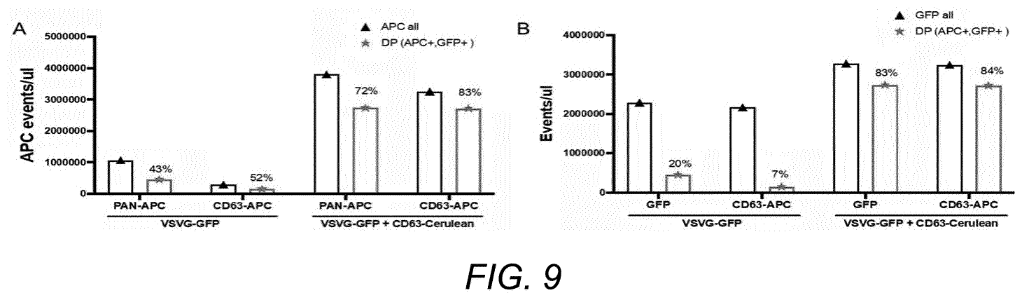

Referring to FIG. 8 which shows a FACs imaging plot of Adenomatous polyposis coli (APC) protein against GFP. Each column from left to right provides for unstained, Pan-APC Ab, CD63-APC Ab, CD9-APC Ab, CD81-APC Ab. The top row shows VSVG-GFP and the bottom row shows VSVG-GFP and CD63-Cerulean.

Two groups were tested. Group 1 comprised pLEX-VSVG-GFP transfection of HEK-293T cells for 48 h. (top row).

Group 2 comprised pLEX-VSVG-GFP and pLEX-CD63-Cerulean co-transfection of HEK-293T cells for 48 h (bottom row).

When VSVG was co-transfected with CD63, each showed clear co-localization with the other by cellstream detection as highlighted by the section surrounded by a (red) box.

When there is only VSVG-GFP transfection, on calculating the double positive events out of APC positive events, only around half of both signals overlap.

In the case of co-transfection, on calculating the double positive events out of APC positive events, around 80% of both signals overlap.

Referring to FIG. 9, which graphically plots that observed by the FACs image of FIG. 8, with events per μl (Y-axis) against VSVG-GFP alone and VSVG-GFP+CD63-cerulean (X-axis).

Referring specifically to FIG. 9A APC events were of particular interest with all APC (▴; left) is shown compared with DP APC+, GFP+ (★; right) for PAN-APC and CD63-APC.

Referring specifically to FIG. 9B events were of particular interest with all GFP (▴; left) is shown compared with DP APC+, GFP+ (★; right) for GFP and CD63-APC.

When there is only VSVG-GFP transfection, on calculating the double positive events out of GFP positive events, then around 7-20% overlap of both signals was observed. This is because the majority of the EVs were GFP positive while only a small part of the EVs were CD63 positive.

In case of co-transfection, upon calculating the double positive events out of GFP positive events, more than 80% overlap of both signals was observed.

The inventors have surprisingly found that VSVG co-localizes with CD63 when co-transfected. As such, both constructs (i.e., one comprising VSVG and the other comprising EV protein (preferably exosomal protein plus optionally intein and cargo)) can be found on the same particle, wherein the particle may be a cell and/or EV depending on the parameters selected.

The inventors have found that co-localisation is key for functional delivery of cargo. More than 83% co-localization of CD63 and VSV-G was achievable using the constructs herein disclosed, which explains why the system worked so well, even at relatively low doses. This is most surprising as existing approaches are believed to only achieve about 70% co-localisation (of an endosomal escape enhancer and an EV protein) and require much higher doses to effectively deliver cargo.

### Example 8: Confirming Functional Delivery of Cre by EVs Having One Construct

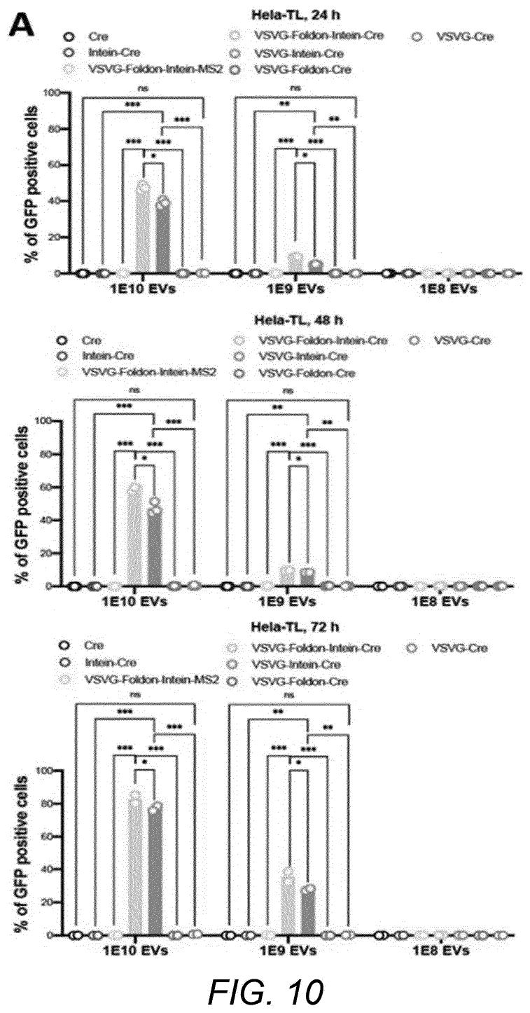

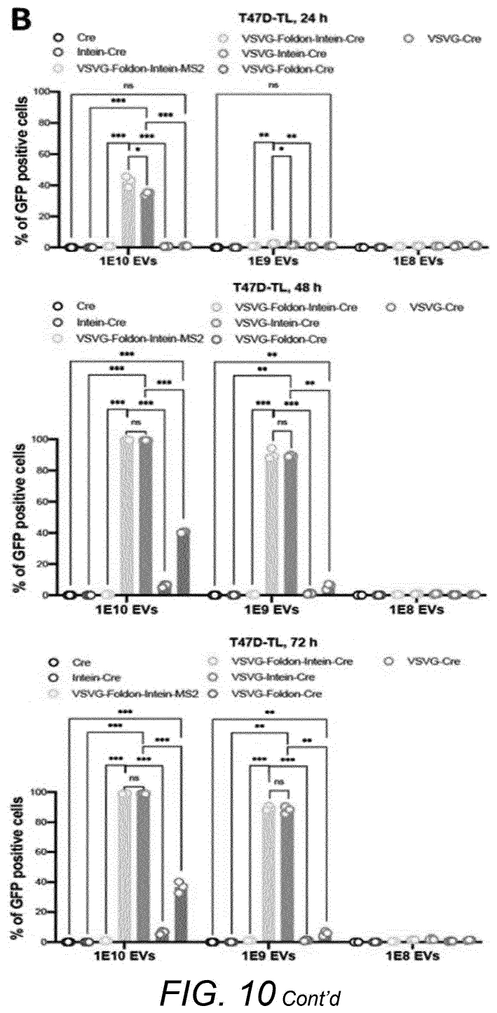

Referring to FIG. 10 plots show percentage (%) GFP positive cells (Y-axis) against EV concentrations 1E10, 1E9, 1E8 (X-axis) comprising each of the following constructs: cre (black circle; far left); intein-cre (dark blue circle; mid left); VSVG-foldon-intein-MS2 (light blue circle; inner left); VSVG-Foldon-Intein-Cre (green circle; innermost); VSVG-Intein-Cre (purple circle; inner right); VSVG-Foldon-Cre (red circle; mid right); VSVG-Cre (brown circle; far right).

Referring specifically to FIG. 10A a series of graphs showing the effect over three timepoints (24 h, 48 h and 72 h) in Hela-TL reporter cells.

Referring specifically to FIG. 10B a series of graphs showing the effect over three timepoints (24 h, 48 h and 72 h) in T47D-TL reporter cells.

Here the prepared EVs were added by transient transfection directly to reporter cells. The inventors found the EVs showed dose and time dependent recombination in Hela and T47D traffic-light cells.

The inventors surprisingly found that a VSVG construct, wherein the VSVG has an EV membrane locating domain (or exosomal membrane locating domain) may suitably replace the EV protein (or exosomal protein) component, meaning that one construct could achieve the same end as the double-stable construct as herein also described. The inventors postulate here that as only one construct is needed the issues associated with a double-stable construct as herein discussed, are avoided. A slight elevation in the % of GFP positive cells was observed when Foldon was included in the construct.

The inventor's postulate that, while Foldon is not strictly necessary for the single construct (VSVG-intein-Cre) to function, inclusion of a multimerization domain like foldon would improve the protein folding, leading to a more stable protein construct and they attribute the elevated GFP positive cells to this phenomenon.

The inventors further surmise that the foldon may be helped VSV-G to form a good conformation and so function better. Western Blot data (not shown) indicated that the foldon increased both VSV-G and cargo expression in the isolated EVs.

As hereinbefore discussed, including, for example foldon, into the construct has no guarantee of success in practice due to engineering issues that may arise (particularly when included as part of a fusion protein)—and the protein of interest (here the foldon) may lose the desired activity.

As such a single construct comprising VSVG-intein-Cre and alternatively VSVG-foldon-intein-Cre has surprisingly been found to function in practice.

As a result of a single construct comprising VSVG-intein-Cre, and optionally foldon, has the advantage of avoiding the risk of protein misfolding, toxic protein aggregation, translational errors, imperfect association and dissociation between the proteins.

It will be appreciated that while Cre is a reporter protein it is representative of what can realistically be expected of delivery of a therapeutic protein.

### Example 10: Diversity of EVs Having One Construct Useful in the Delivery of Cre

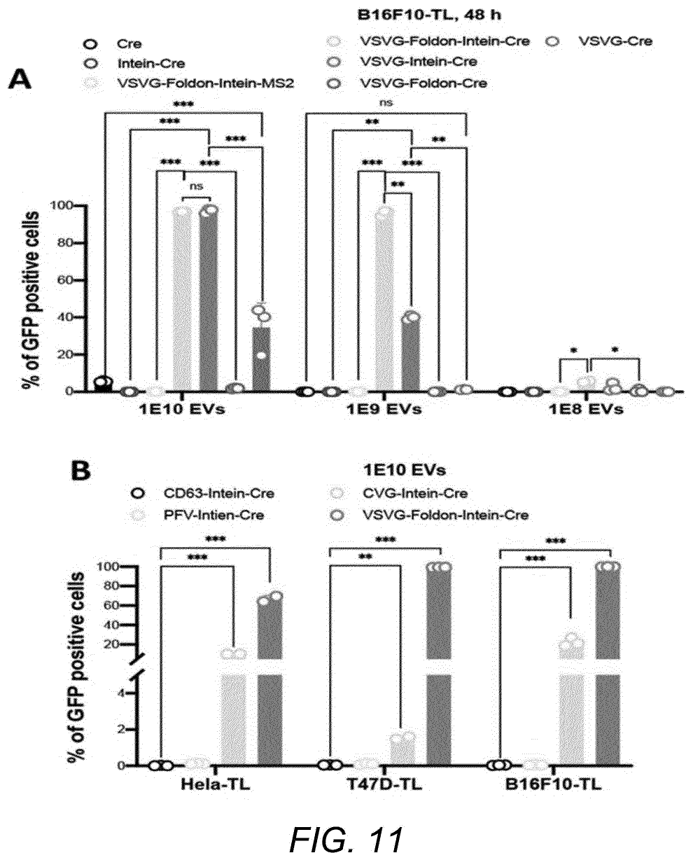

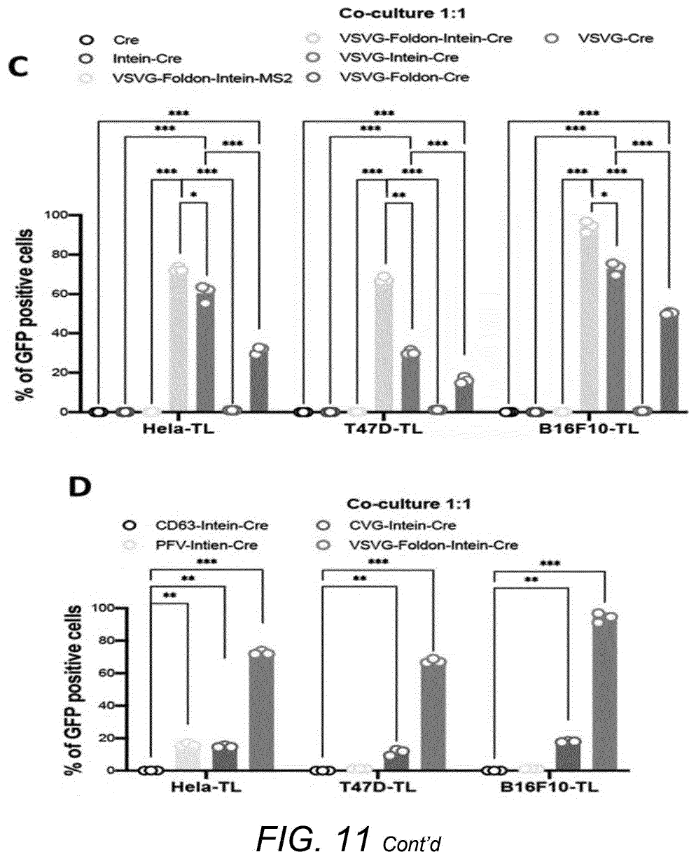

Referring to FIG. 11, which shows percentage (%) GFP positive cells (Y-axis) for various protein constructs of interest across certain reporter cell types.

Referring specifically to FIG. 11A which shows the percentage (%) GFP positive cells (Y-axis) for the following constructs: cre (black circle; far left); intein-Cre (dark blue circle; mid left-hand side); VSVG-foldon-intein-MS2 (light blue circle; inner left-hand side); VSVG-foldon-intein-Cre (green circle; innermost); VSVG-intein-Cre (purple circle; inner right); VSVG-foldon-Cre (red circle; mid right); and VSVG-Cre (brown circle; far right) at an EV concentration of 1E10, 1E9 and 1E8 (X-axis) in reporter cells B16F10.

Referring specifically to FIG. 11B which shows the percentage (%) GFP positive cells (Y-axis) for the following constructs: CD63-intein-cre (black circle; far left); PFV-intein-Cre (light blue circle; mid left-hand side); CVG-intein-Cre (green circle; inner right-hand side); VSVG-foldon-intein-Cre (purple circle; far right) at an EV concentration of 1E10 across different reporter cells Hela-TL, T47D-TL and B16F10-TL (X-axis).

Referring specifically to FIG. 11C which shows the percentage (%) GFP positive cells (Y-axis) for the following constructs: cre (black circle; far left); intein-Cre (dark blue circle; mid left-hand side); VSVG-foldon-intein-MS2 (light circle; inner right-hand side); VSVG-foldon-intein-Cre (green circle; innermost); VSVG-intein-Cre (purple circle; inner right); VSVG-Foldon-Cre (red circle; mid right); VSVG-Cre (brown circle; far right) wherein the EV-producing cells were co-cultured with one of the reporter cells Hela-TL, T47D-TL and B16F10-TL (X-axis) at a cell-to-cell ratio of 1:1.

Referring specifically to FIG. 11D which shows the percentage (%) GFP positive cells (Y-axis) for the following constructs: CD63-intein-cre (black circle; far left); PFV-intein-Cre (light blue circle; mid left-hand side); CVG-intein-Cre (green circle; inner right-hand side); VSVG-foldon-intein-Cre (purple circle; far right) wherein the EV-producing cells were co-cultured with one of the reporter cells Hela-TL, T47D-TL and B16F10-TL (X-axis) at a cell-to-cell ratio of 1:1.

### Example 11: EV Endocytosis Profile

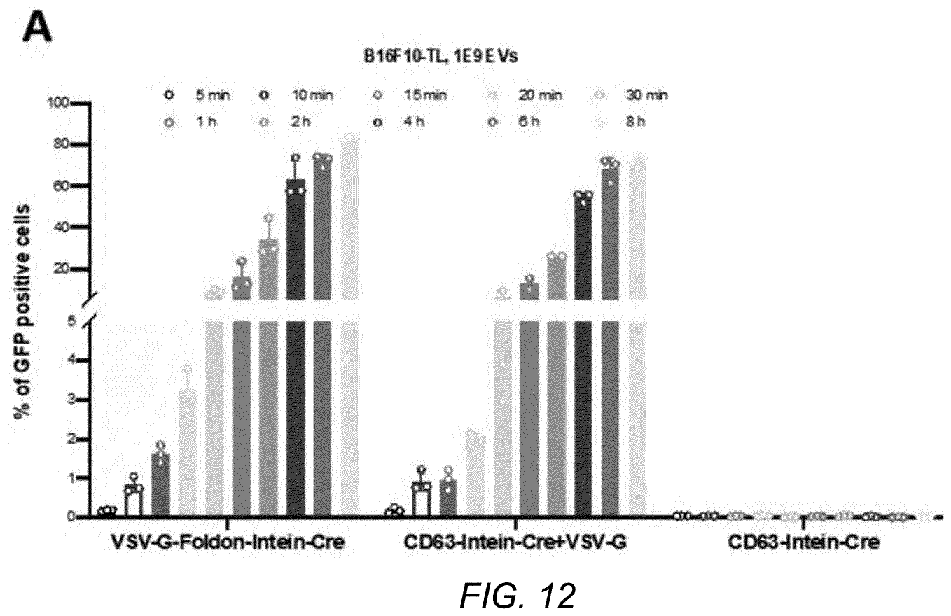

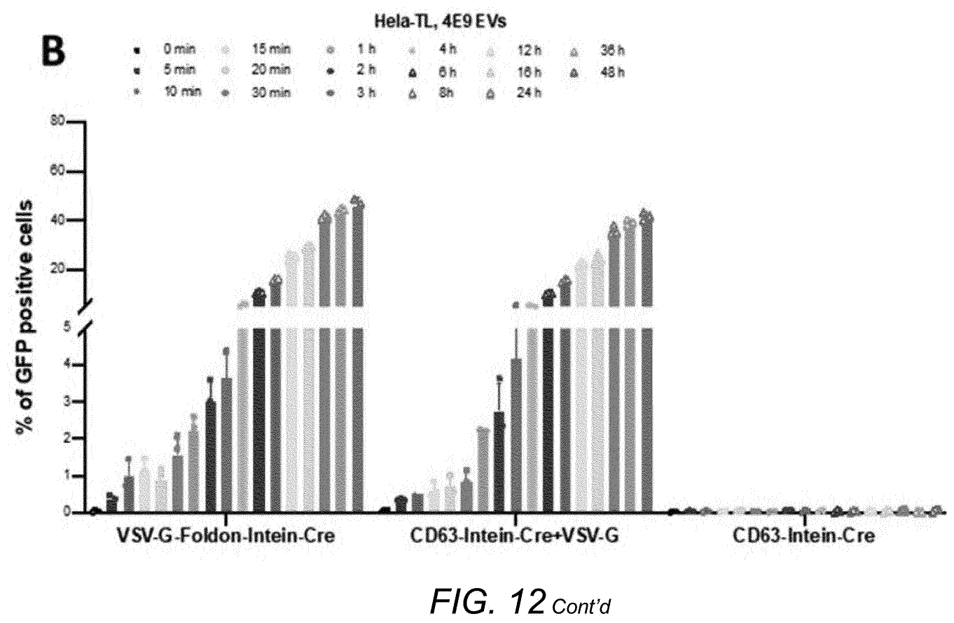

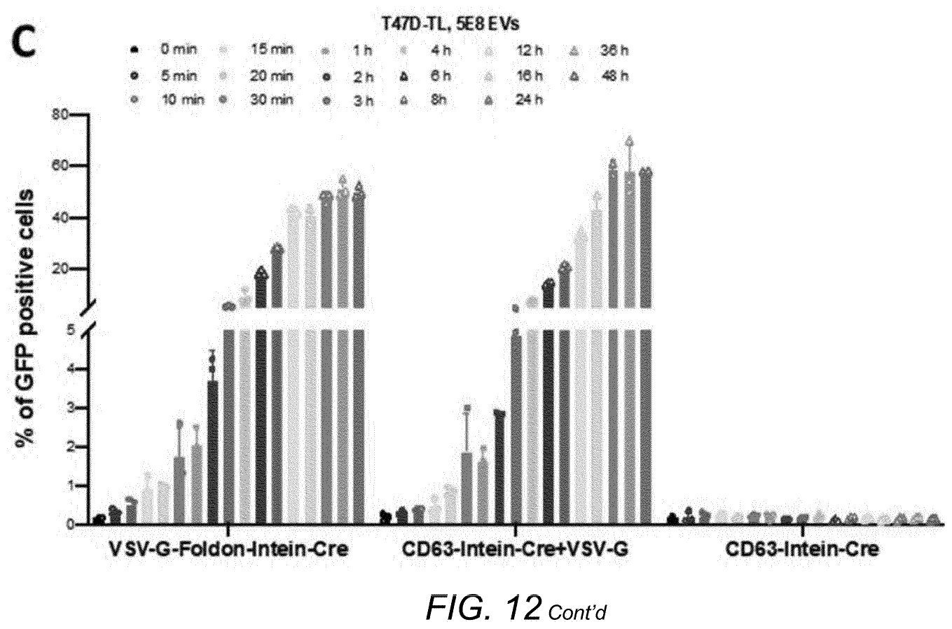

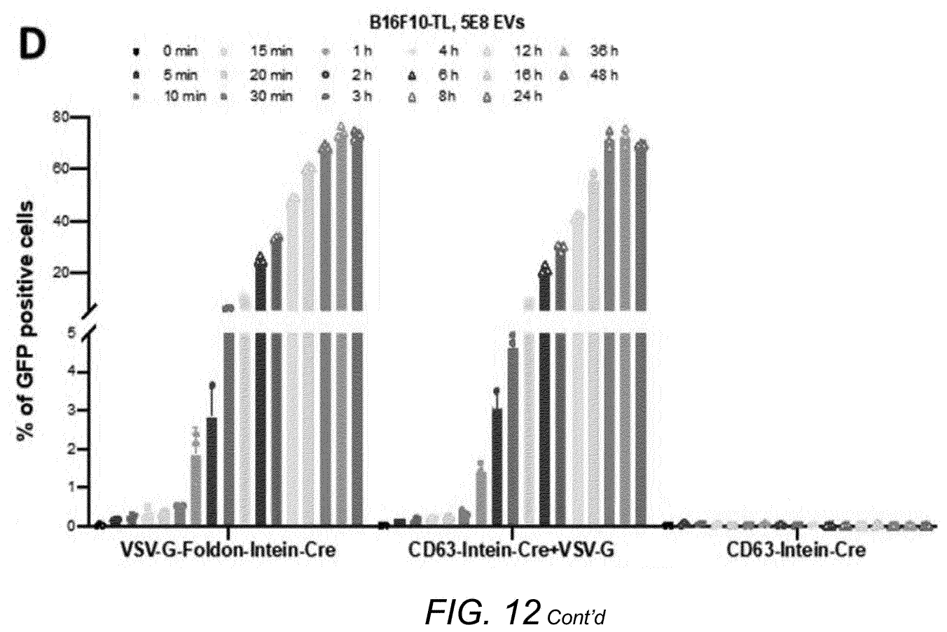

Referring to FIG. 12 which shows the percentage (%) GFP positive cells (Y-axis) for various protein constructs of interest (X-axis) in different reporter cell types at the following timepoints: 0 min, 5 min, 10 min, 15 min, 20 mins, 30 min, 1 hr, 2 hr, 4 hr, 6 hr, 8 hr, 12 hr, 16 hr, 24 hr, 36 hr, 48 hr

Referring specifically to FIG. 12A which shows the percentage (%) GFP positive cells (Y-axis) for the protein constructs VSVG-foldon-intein-Cre, CD63-intein-Cre+VSVG and CD63-intein-Cre (X-axis) in B16F10 reporter cells at a concentration of 1E9.

Referring specifically to FIG. 12B which shows the percentage (%) GFP positive cells (Y-axis) for the protein constructs VSVG-foldon-intein-Cre, CD63-intein-Cre+VSVG and CD63-intein-Cre (X-axis) in B16F10 reporter cells at a concentration of 5E8.

Referring specifically to FIG. 12C which shows the percentage (%) GFP positive cells (Y-axis) for the protein constructs VSVG-foldon-intein-Cre, CD63-intein-Cre+VSVG and CD63-intein-Cre (X-axis) in Hela-TL reporter cells at a concentration of 4E9.

Referring specifically to FIG. 12D which shows the percentage (%) GFP positive cells (Y-axis) for the protein constructs VSVG-foldon-intein-Cre, CD63-intein-Cre+VSVG and CD63-intein-Cre (X-axis) in T47D-TL reporter cells at a concentration of 5E8.

The EV endocytosis may happen immediately after adding to the cells. Because even removing the EVs after adding for 5 min, some cells turned to GFP positive and could be picked up under the microscope. The signals could be detected as early as 10 min and continuously increased up to 24 h or 36 h, indicating the process of EV uptake and endocytosis lasted at least 24 h to 36 h after adding EVs. For B16F10 cells, if 2 times of EVs, 1E9 instead of 5E8, were added, the uptake at indicated time points increased accordingly, indicating again the dose dependent EV uptake and subsequent functional protein delivery.

### Example 12: Delivery of Alternative Cargo, Cas9

In this experiment the stop-light cells were transfected with guide RNA (gRNA) or were not transfected with guide RNA (i.e., No gRNA) for 24 h and then the corresponding EVs were added into the cells. After adding EVs for 72 h, FACS was carried out to check the GFP positive cells.

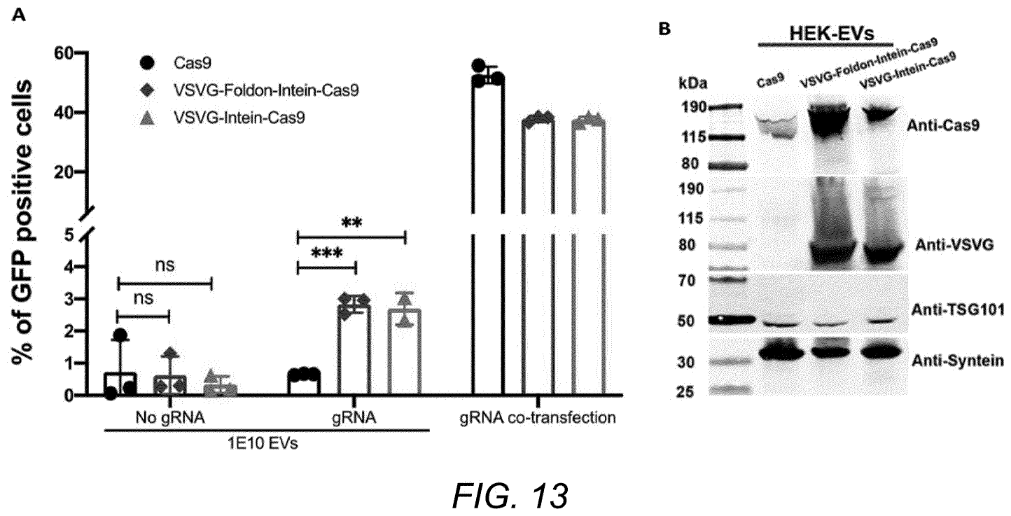

Referring to FIG. 13, this shows the delivery of alternative cargo from certain protein constructs graphically by plotting percentage (%) GFP positive cells and as a western blot.

Referring specifically to FIG. 13A, this shows percentage (%) GFP positive cells (Y-axis) for the following protein constructs: Cas9 (^{•}), VSVG-Foldon-Intein-Cas9 (^{♦}), VSVG-Intein-Cas9 (^{▴}) with and without gRNA at a concentration of 1E10 and when co-transfected with gRNA. Co-transfection with gRNA meant co-transfection of the corresponding plasmids (see above) was with the gRNA respectively, serving as the positive control of this experiment.

Referring specifically to FIG. 13B, this shows a western blot (WB) indicating a ladder to the left-hand side of the WB. The columns from left to right are Cas9, VSVG-Foldon-Intein-Cas9, VSVG-intein-Cas9.

FIG. 13 demonstrates that alternative cargo other than Cre may also equally benefit from the protein construct as herein disclosed. In particular, the inventors have shown that it is possible to functionally deliver super-large proteins, such as Cas9, into recipient cells.

Also, CRISPR-associated endonuclease Cas9 is an example of a nucleic acid binding protein. The data of FIG. 13 show that Cas9 binds to and drags in additional RNAs along with it (e.g., gRNA).

A similar experiment has also been performed to test the CD63-Intein-Cas9 plasmid (data not shown). Similar results are proposed.

### Example 14: Delivery of Alternative Cargo, Super Suppressor of NF-κB by a Double-Stable Construct

For this experiment, HEK-Blue (NF-kB response) reporter cells were used. Without stimulation, the NF-kB will be bound by IKBA, and cannot be translocated to the nucleus to drive the expression of firefly luciferase. Once stimulated by something, such as TNFa or LPS, IKBA will be phosphorylated and degraded, dissociated from the NF-kB complex so that NF-kB can be translocated to the nucleus to drive the expression of firefly luciferase. The super suppressor (SS) of NF-kB is a mutated version of IKBA, with the combination of 2 mutations (S32A and S36A). The mutated IKBA can bind to NF-kB but cannot be phosphorylated upon stimulation, thereby decreasing the nuclear translocation of NF-kB. Reporter cells worked to respond to stimulation, such as TNFa (data not shown). The SS constructs worked to decrease NF-kB nuclear translocation under stimulation in reporter cells (data not shown). The engineered EVs functionally delivered SS to reporter cells to decrease NF-kB nuclear translocation under stimulation in reporter cells. (data not shown). The EVs were added to the reporter cells for 48 h first and then the TNFa (10 ng/ml) was added into the cells. 6 h after stimulation, firefly signals were checked by machine.

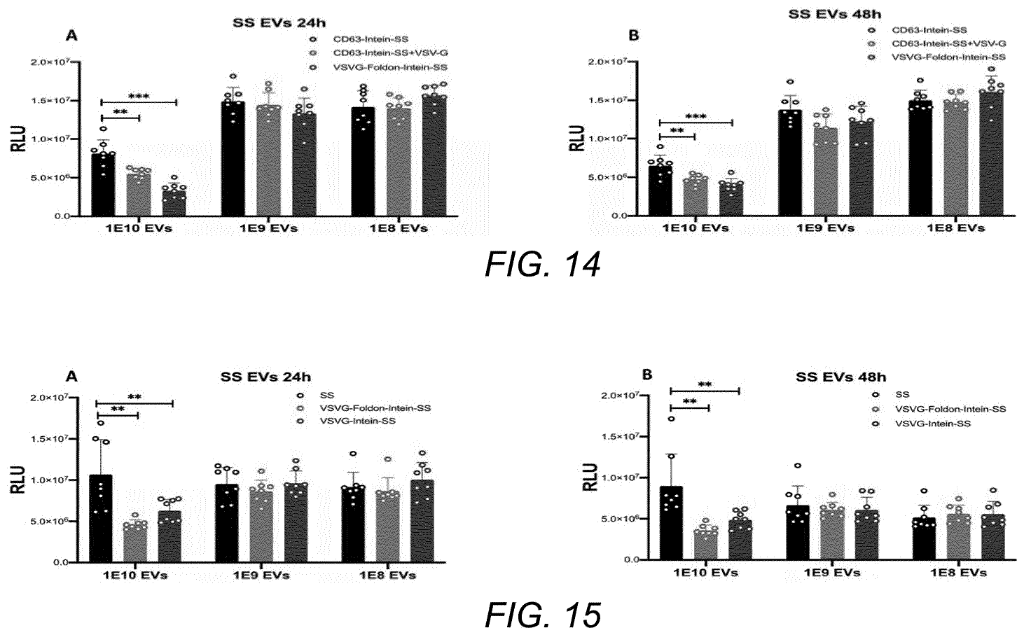

Referring to FIG. 14, this shows RLU (Y-axis) for various constructs at a concentration of 1E10, 1E9 and 1E8 (X-axis).

The corresponding EVs were added into the reporter cells for 24 h or 48 h respectively, and then TNFa (10 ng/ml) was added to stimulate the cells. After 6 h stimulation, firefly luciferase signals were measured.

Referring specifically to FIG. 14A shows the RLU (Y-axis) for the following constructs: CD63-Intein-SS (Black circle; left side); CD63-Intein-SS+VSVG (red circle; middle); VSVG-Foldon-Intein-SS (Blue circle; right) at a 24 hr timepoint.

Referring specifically to FIG. 14B shows the RLU (Y-axis) for the following constructs: CD63-Intein-SS (Black circle; left side); CD63-Intein-SS+VSVG (red circle; middle); VSVG-Foldon-Intein-SS (Blue circle; right) at a 48 hr timepoint.

Both VFIC and VEDIC EVs worked well to deliver SS proteins in vitro to inhibit NFkB nuclear translocation in reporter cells.

### Example 15: Delivery of Alternative Cargo, Super Suppressor of NF-κB by a Single Construct

Referring to FIG. 15, this shows RLU (Y-axis) for various constructs at a concentration of 1E10, 1E9 and 1E8 (X-axis).

The corresponding EVs were added into the reporter cells for 24 h or 48 h respectively, and then TNFa (10 ng/ml) was added to stimulate the cells. After 6 h stimulation, firefly luciferase signals were measured.

Referring specifically to FIG. 15A shows the RLU (Y-axis) for the following constructs: SS (Black circle; left side); VSVG-Foldon-Intein-SS (red circle; middle); VSVG-Intein-SS (Blue circle; right) at a 24 hr timepoint.

Referring specifically to FIG. 15B shows the RLU (Y-axis) for the following constructs: SS (Black circle; left side); VSVG-Foldon-Intein-SS (red circle; middle); VSVG-Intein-SS (Blue circle; right) at a 48 hr timepoint.

The VSVG-Foldon-Intein-SS and VSVG-Intein-SS EVs (1E10) started to work from 24 h after adding. And with Foldon, the suppression effect of NF-κB activation was more significant. The 1E9 and 1E8 doses did not work.

VFIC EVs worked well to deliver SS proteins in vitro to inhibit NFkB nuclear translocation in reporter cells. With the trimerization domain, Foldon, the SS proteins were better delivered.

### Example 16: Effect of EVs in LPS Induced Inflammation

An LPS induced inflammation model was established and used to assess the EVs as herein described in detail.

C57BL/6 mice were purchased at around 5-week age with 20 g body weight. After at least one-week acclimation to the new environment, the animals were injected (I.P) with the engineered EVs 4 h before injection (I.P) of LPS (Sigma, USA) at the dose of 7.5 mg/Kg. 6 h after LPS induction, the engineered EVs were injected (I.P) once more to boost the intracellular delivery of the protein cargos by EVs. The survival and body weight of the mice with LPS induction was recorded for 2 days. 48 h after LPS induction, the mice were euthanized, and blood was sampled from heart puncture. Then the mice were sacrificed, and main organs (liver, lung, spleen and kidney) were harvested to be fixed with PFA for slide making. H&E (haematoxylin and eosin) stain was performed to check the damage extent of the organs induced by LPS. The damage of the tissues was evaluated by professional pathologist and histological scores of the tissues were given accordingly.

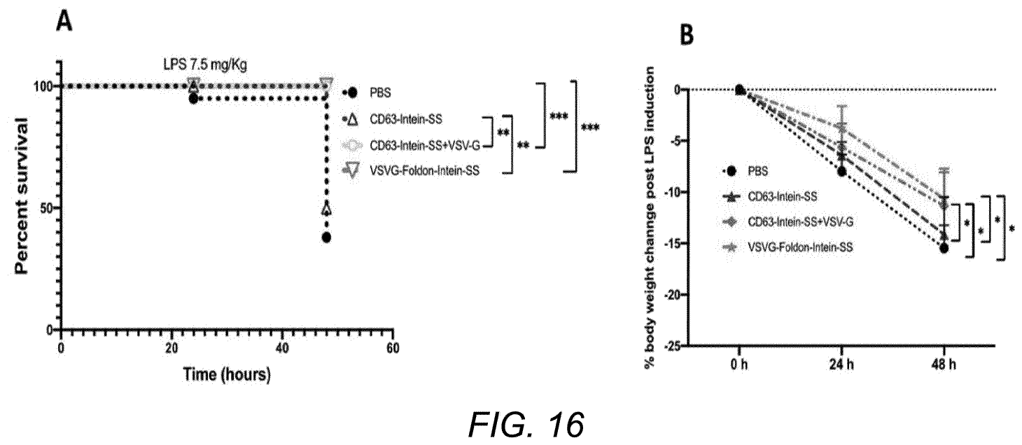

Referring to FIG. 16, this plots the effect of certain constructs over time.

Referring specifically to FIG. 16A plots the percent survival to LPS 7.5 mg/kg after administration of the following: PBS ( [symbol] ), CD63-Intein-SS (*^{Δ}), CD63-intein-SS+VSVG ( [symbol] ) VSVG-Foldon-Intein-SS ( [symbol] ) over time.

Referring specifically to FIG. 16B plots the percentage (%) body weight change post LPS induction and after administration of the following: PBS ( [symbol] ), CD63-Intein-SS ( [symbol] ), CD63-intein-SS+VSVG ( [symbol] ), VSVG-Foldon-Intein-SS ( [symbol] ) over time.

Therapeutic EVs (CD63-Intein-SS+VSV-G and VSVG-Foldon-Intein-SS) significantly prevented the mice from LPS induced death.

Therapeutic EVs (CD63-Intein-SS+VSV-G and VSVG-Foldon-Intein-SS) significantly reduced the mice body weight loss from LPS induction.

### Example 17: Effect of EVs on Tumour Progression In Vivo

First an Intra-tumour injection model (e.g., melanoma model) was established and used to assess the effect of EVs as herein disclosed on tumour progression/suppression.

C57BL/6 mice were ordered at around 5-week age with 20 g body weight. The animals were acclimated to their new surroundings at least one week before the experiment. B16F10-TL cells were harvested and resuspended in PBS and then the animals were inoculated with the cells subcutaneously at the number of 0.5 million per mouse. After 10 days of inoculation, when obvious tumours were formed, engineered EVs were injected directly into the tumours. The injected volume was 50 μL per mouse with 7.5×1010 EVs. 4 days after intra-tumour injection of EVs, the mice were sacrificed, and tumours were harvested. Half of the tumour tissues were fixed with PFA and sent for slide making and the other half were immersed in lysis buffer (0.1% TritonX-100) for DNA isolation. When the slides with tumoral tissue on were ready, IHC staining for GFP expression was performed and the tissues in lysis buffer were homogenised with tissue analyser machine. 50 μL of the tissue lysate was taken for DNA isolation by using the Maxwell® RSC tissue DNA Kit (Promega, USA).

Further to the study performed above, an intracerebroventricular (ICV) injection of the EVs comprising one or more of the constructs as hereinbefore described in Example 1 was administered to murine subjects at around 1E10 EVs per mouse: In particular, EVs comprising CD63-Intein-Cre, CD63-Intein-Cre+VSV-G and VSVG-Foldon-Intein-Cre constructs were administered for evaluation.

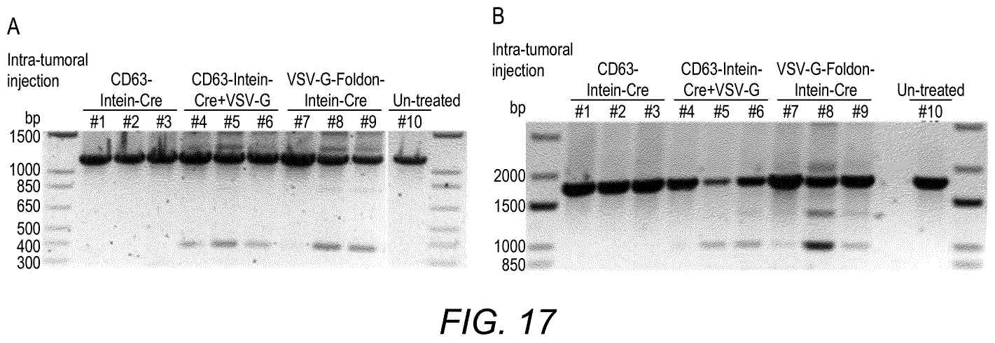

Referring to FIG. 17, this shows a western blot (WB) indicating a ladder to the left-hand side of the WB. The columns from left to right showing the various protein constructs tested and un-treated (control) on the far right.

Referring specifically to FIG. 17A there is shown a WB. The columns from left to right showing the constructs as follows: CD63-intein-Cre, CD63-intein-Cre+VSVG and VSVG-Foldon-intein-Cre. Primer 1 and Primer 2 (designs not shown) were used.

Where Cre proteins were delivered into tumour cells in vivo, a smaller band (around 370 bp) was observed by using primer 1 and primer 2.

Referring specifically to FIG. 17B there is shown a WB. The columns from left to right showing the constructs as follows: CD63-intein-Cre, CD63-intein-Cre+VSVG and VSVG-Foldon-intein-Cre. Primer 1 and Primer 3 (designs not shown) were used.

Where Cre proteins were delivered into tumour cells in vivo, a smaller band (around 1000 bp) was observed by using primer 1 and primer 3.

VEDIC and VFIC EVs were confirmed to work well to deliver Cre proteins in vivo.

### Example 18: Effect of EVs In Vivo

An intracerebroventricular (ICV) injection of PBS (control) or Extracellular vesicles (EVs) or exosomes comprising constructs according to the disclosure were injected into mice subjects. After a period (about 1 week after injection), the organs were harvested (e.g., the brain). Post-harvest, IHC staining was performed, and RFP expression checked.

Tissue samples were prepared for sectioning and analysis by perfusion with 4% PFA and left overnight. The samples were then dehydrated and embedded into paraffin wax. Once fixed, 5 μm paraffin sections were obtained.

To stain, a primary antibody: Anti-RFP (600-401-379, Rockland, 1:400) and a secondary Ab: Alexa Fluor-633 goat anti-rabbit (A-21070, Thermo Scientific, 1:400) were used.

The confocal microscope LSM780 with an apochromatic lens (LD LCI Plan-Apochromat 25×/0.8 Imm Korr DIC M27) was used to capture light corrected images showing the two stains DAPI blue (nucleus) and tdTomato (Cre) in the image plane together.

The Inventors postulate observation of RFP expression in different regions of the brain post injection. Engineered EVs (in particular, CD63-Intein-Cre+VSV-G and VSVG-Foldon-Intein-Cre), demonstrated similar activity (not shown) to that observed from the intra-tumour injection model previously described.

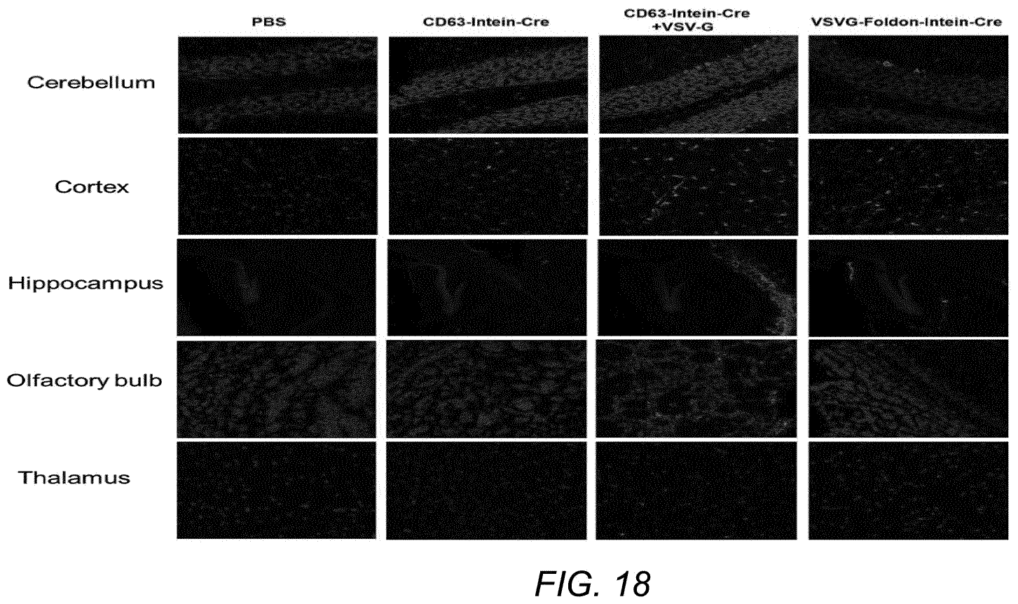

Referring to FIG. 18, this shows a series of confocal microscopy images post administration and harvest of organs as hereinbefore described. Columns from left to right show the EV containing protein constructs used: CD63-intein-Cre; CD63-intein-Cre+VSVG and VSVG-foldon-intein Cre. Far left is the control (PBS). Sections were taken (from the top down) from the following:

cerebellum (top row); cortex (second from top row); hippocampus (middle row); olfactory bulb (second from bottom row); and thalamus (bottom row). Dapi Blue depicts the cells and the tdTomato shows the presence of the cargo (delivered by the EVs having the protein constructs tested) into the cytosol of the cells.

The inventors found that EVs comprising CD63-intein-cre were effective at delivering Cre (e.g., cargo) particularly to the cerebellum and cortex of the brain (as shown by the red dots). Addition of a further protein construct into the EV for an endosomal escape enhancer (e.g., VSV-G) improved the delivery of the cargo as observed as elevated tdTomato (red dots) across all sections of the brain and was particularly effective at delivering cargo to the hippocampus.

This confirms that use of an endosomal escape enhancer is important for improved delivery of cargo from an EV to the site of action. The improved effect observed is thought to be due to the combination of a release system (intein) and the endosomal escape enhancer.

Also confirmed is that, in an embodiment where the EV has two protein constructs, it is stably and functionally formed from the double-stable cell when a release system and an endosomal escape enhancer is used.

Also, with a single construct, having an endosomal escape enhancer (e.g., VSV-G), multimerization domain (e.g., foldon), and release system (e.g., intein), the cargo is effectively and functionally released across all sections of the brain. This is comparable to an EV having two separate protein constructs where one comprises an EV protein (e.g., CD63), a release system (e.g., intein) and the cargo and the other protein construct having the endosomal escape enhancer.

Being able to combine the endosomal escape enhancer into the same protein construct as the release system and the cargo is particularly advantageous, since the protein construct is smaller in size, meaning greater stability of the construct in the EV membrane and lower chances of the protein construct from dissociation from the membrane. Also, where the cargo is lumen side and/or associated with the inner leaflet of the EV membrane, even greater stability is imparted, since the cargo and the endosomal escape enhancer are not in steric competition with each other.

### Example 19-VSV-G Boosts Endosomal Escape Following Receptor-Mediated Endocytosis of VSV-G-Engineered Evs into Recipient Cells

To ascertain the role of VSV-G in endosomal escape and endocytosis, mutant versions that have been described to lack fusogenic (VSV-G P127D) or LDL-R receptor binding capacity (VSV-G K47Q), respectively were introduced.

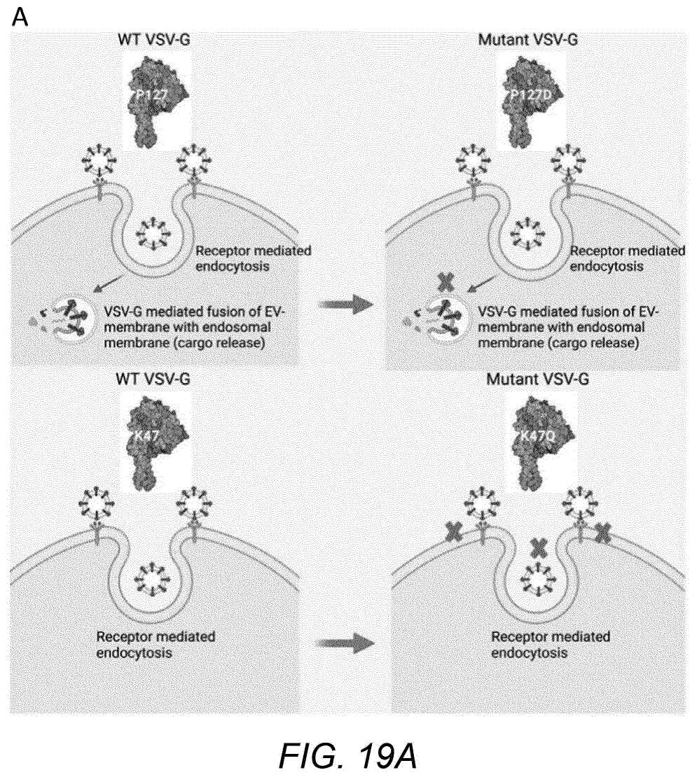

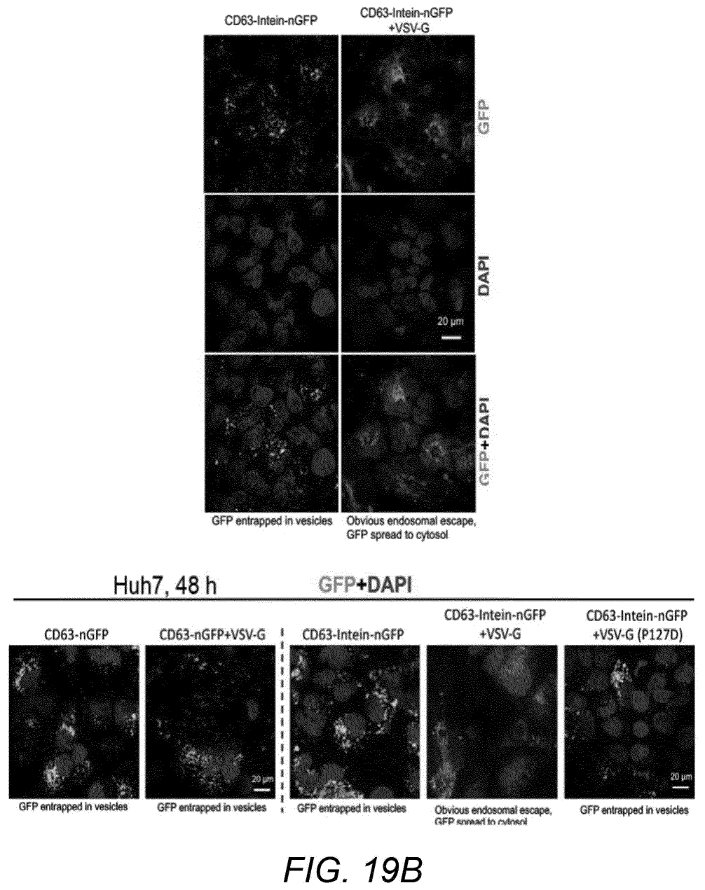

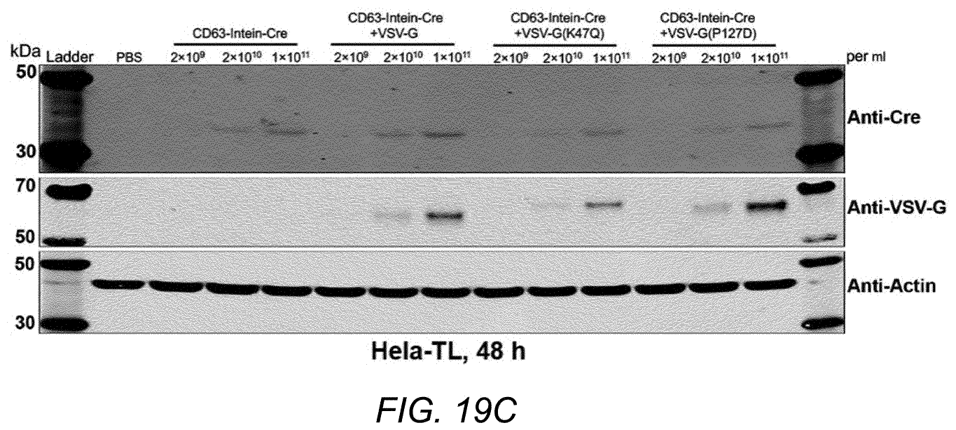

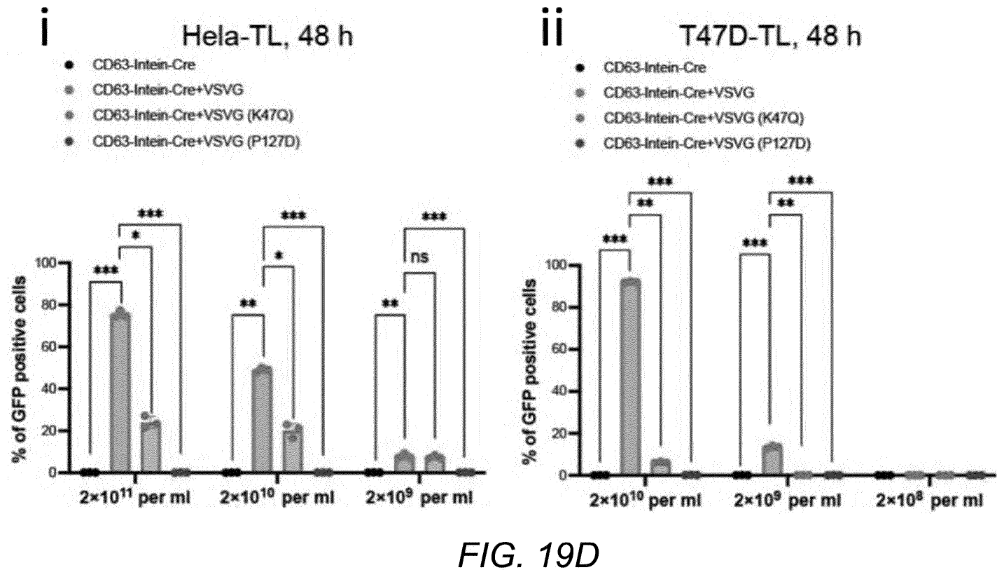

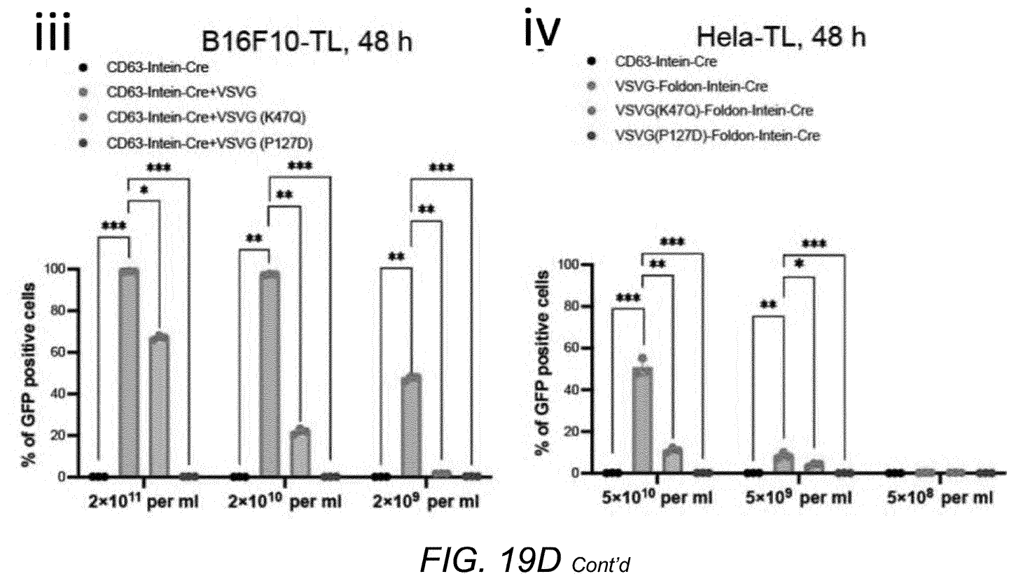

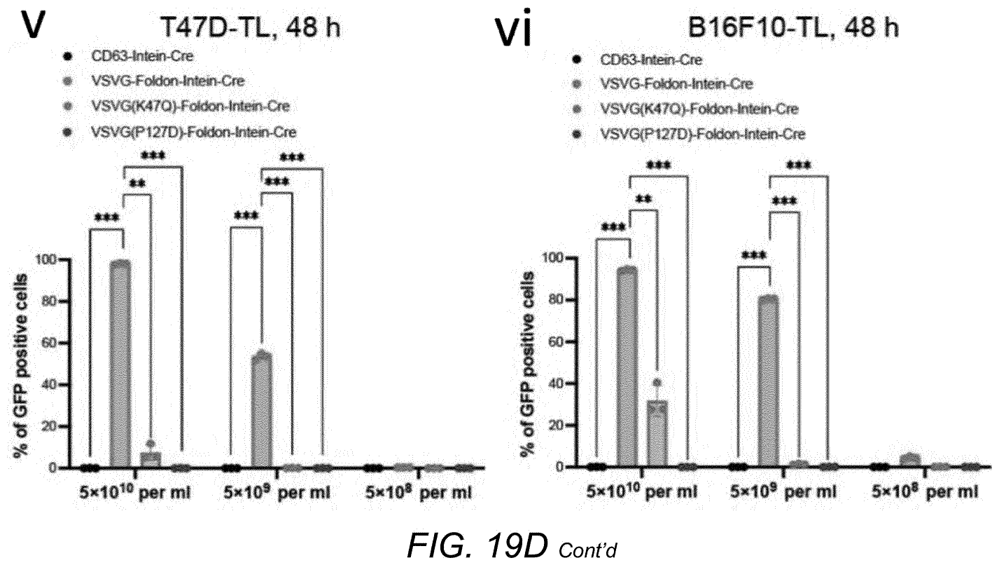

Referring to FIG. 19, VSV-G boosts endosomal escape following receptor-mediated endocytosis of VSV-G-engineered EVs into recipient cells. FIG. 19A demonstrates the properties of the two VSV-G mutants: VSV-G P127D loses the capacity to mediate fusion between the EV- and endosomal membranes and VSV-G K47Q is unable to bind to LDLR on the cell surface.

For imaging, Cre was replaced by mNeonGreen (nGFP) to generate a CD63-Intein-nGFP construct, which was co-transfected with VSV-G to produce EVs that were added to Huh7 cells. Confocal microscopy was used to evaluate the uptake of the vesicles 48 h after incubation and showed a punctate distribution of GFP in recipient cells in the absence of VSV-G, indicating endosomal entrapment.

However, as is demonstrated by FIG. 19B, when VSV-G was included, the GFP signal diffused into the cytosol, suggesting endosomal escape. FIG. 19B refers to confocal immunofluorescence and demonstrates the subcellular distribution of nGFP in the presence or absence of wild type or P127G VSV-G and intein in engineered EVs in Huh7 cells. Scale bar=20 μm. Of particular note, and depicted in FIG. 19B, when VSV-G P127D was used, GFP instead showed a punctate distribution, confirming that endosomal escape was mediated by VSV-G.

Intriguingly, as can be seen in FIG. 19B, GFP was observed in punctate form when CD63-nGFP was co-transfected with VSV-G, suggesting that this is retained in endosomes and confirming the role of intein for intracellular delivery of protein cargo by engineered EVs observed for Cre delivery.

FIG. 19C shows a western blot evaluation of protein levels of Cre and VSV-G in Hela-TL cells after adding engineered EVs with wild type, P127D or K47Q VSV-G.

FIG. 19D, i-iii shows the percentage of GFP positive Hela-TL, T47D-TL and B16F10-TL cells after adding wild type, P127D or K47Q VSV-G CD63-Intein-Cre EVs, as evaluated by flow cytometry.

FIG. 19D, iv-vi shows the percentage of GFP positive Hela-TL, T47D-TL and B16F10-TL cells after adding CD63-Intein-Cre EVs or wild type, P127D or K47Q VSV-G Foldon-Intein-Cre EVs, as evaluated by flow cytometry. Two-way ANOVA test was used for analysis and the data are shown as mean+SD, * p<0.05; ** p<0.01; *** p<0.001.

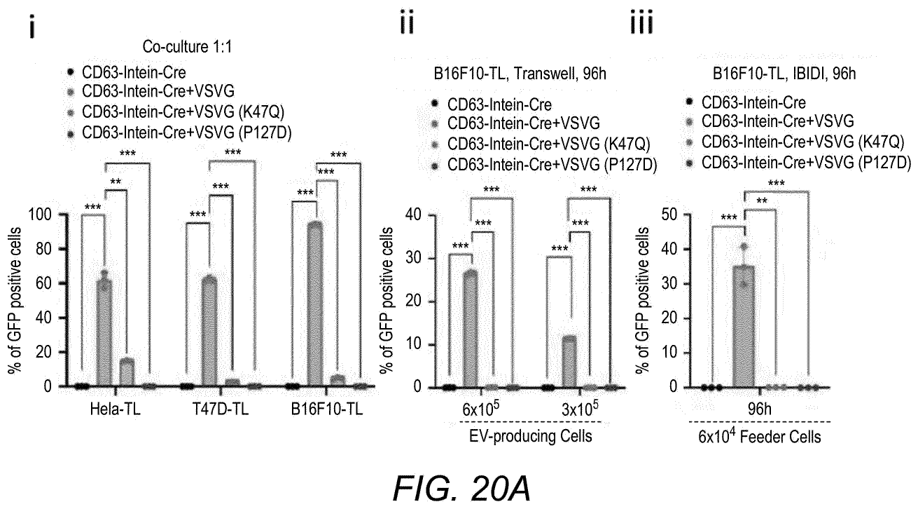

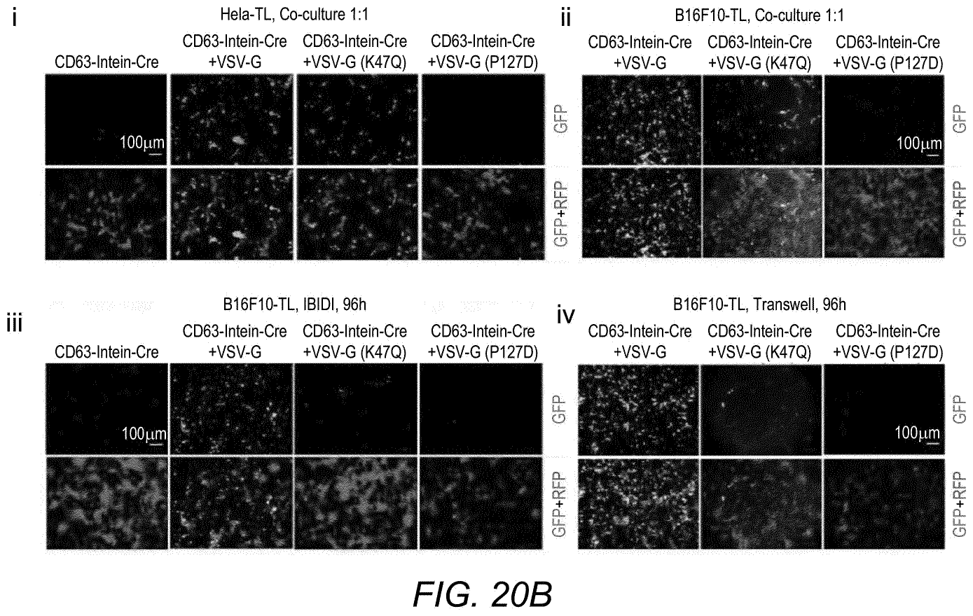

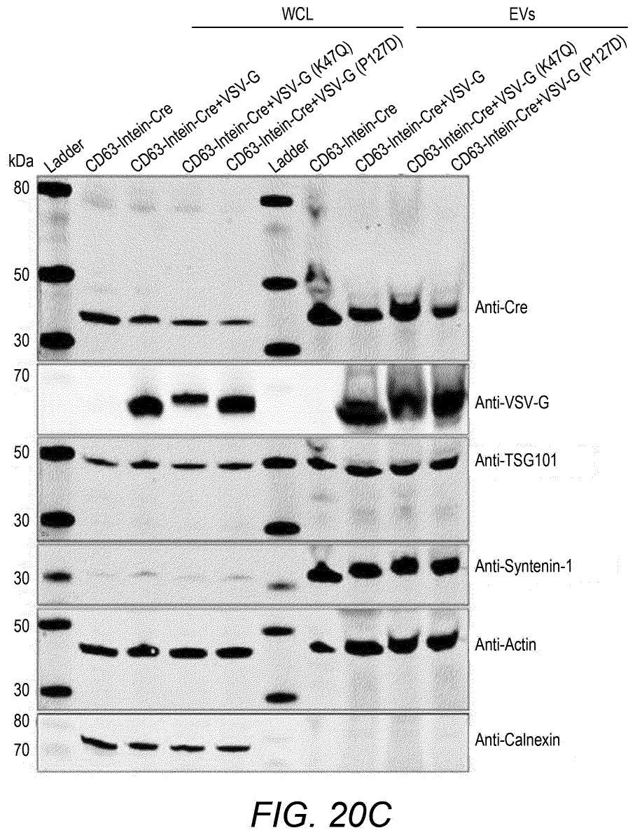

Next, the two mutant VSV-G constructs were used for Cre protein delivery, and it was found that these both decreased Cre protein levels detected in Hela-TL recipient cells, which is demonstrated in FIG. 20A.

As can be seen in FIG. 19D, i-iii and FIG. 20A, the application of the two VSV-G mutants to the VEDIC system, showed significantly decreased recombination with VSV-G K47Q and complete loss of reporter activity with VSV-G P127D in EV treatment, co-culture, IBIDI and transwell assays in different reporter cells.

FIG. 19D, iv-vi and FIGS. 20A and 20B show that these results could be reproduced using the VFIC system to deliver Cre protein. This supported the conclusion that VSV-G functions to facilitate endosomal escape and receptor-mediated endocytosis of engineered EV cargo into recipient cells.

The evaluation of the function of VSV-G to enhance endosomal escape and the receptor-mediated endocytosis pathway for the entry of VSV-G engineered EVs by using P127D and K47D mutants for VEDIC system (co-culture, IBIDI and transwell assays) is demonstrated in FIG. 20A. FIG. 20A, i depicts a co-culture assay to show the recombination efficiency of different engineered EVs in cell-cell contact dependent manner. VSV-G (K47Q) and VSV-G (P127D) mutants were included to the EV-producing cells and demonstrated decreased and abolished effects respectively in recipient cells.

FIG. 20A, ii shows the percentage of recombined GFP positive cells evaluated by transwell assay in B16F10-TL cells. VSV-G (K47Q) and VSV-G (P127D) mutants were included to the EV-producing cells and the results were similar to that got from co-culture assay. Indicated numbers of EV-producing cells and 5×104 B16F10-TL cells were used for the assay in 24-well plate.

FIG. 20A, iii also shows the percentage of GFP positive cells evaluated by IBIDI assay in B16F10-TL cells. Indicated number of EV-producing cells and 4×104 reporter cells were used for this assay.

FIG. 20B, i-ii are representative fluorescent images showing the GFP positive cells from co-culture assay in Hela-TL and B16F10-TL cells respectively. Scale bar, 100 μm.

FIG. 20B, iii-iv also shows the GFP positive cells as demonstrated by fluorescent microscopy from IBIDI and transwell assay respectively in B16F10-TL cells. Scale bar, 100 μm.

FIG. 20C depicts western blot images demonstrating the protein expression of mutated VSV-G-related constructs both in WCL of EV-producing cells and the isolated EVs evaluated by WB. Proteins from 5×105 EV-producing cells and 1×1010 engineered vesicles were used for the assay. There were 2 bands when using Cre antibody for constructs with intein, with one band of un-cleaved full-length protein and the other cleaved free protein. TSG101, syntenin-1 and β-actin were used as EV markers while Calnexin was used as cellular marker (endoplasmic reticulum) and should be absent for EV samples.

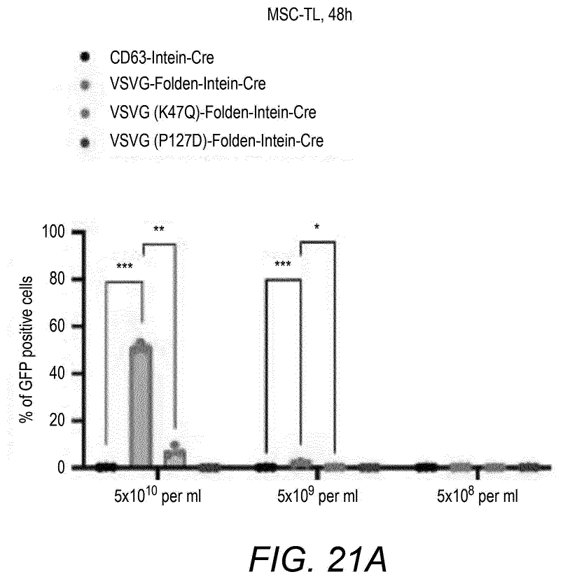

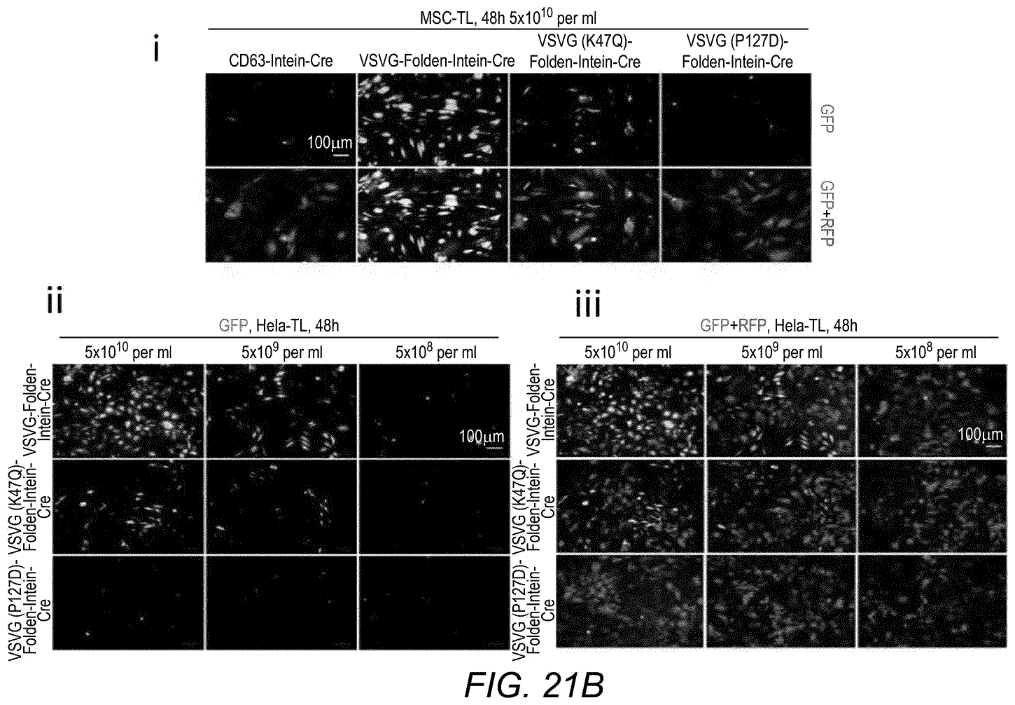

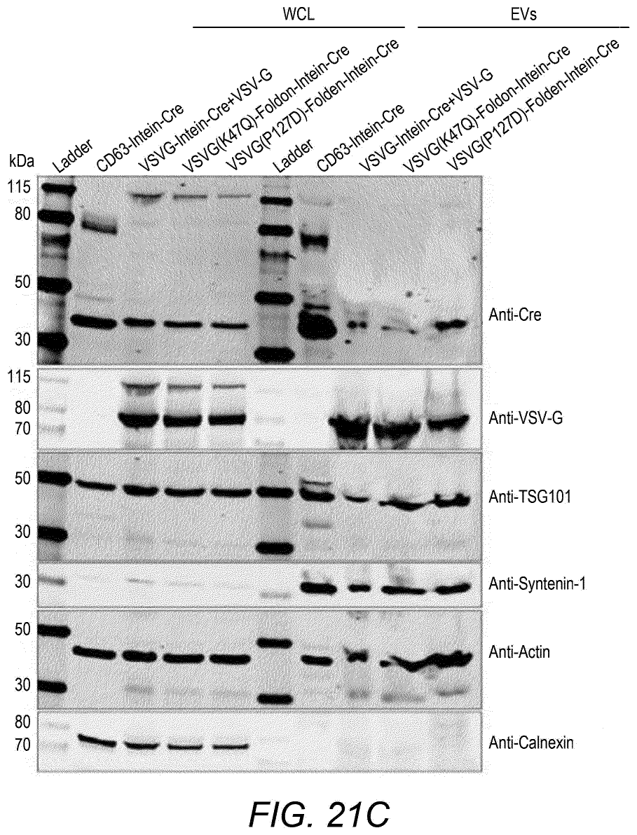

FIG. 21 is an evaluation of the function of VSV-G to enhance endosomal escape and the receptor-mediated endocytosis pathway for the entry of VSV-G engineered EVs by using P127D and K47D mutants for VFIC system (EV-adding assay).

Referring specifically to FIG. 21A, this shows the percentage of recombined GFP positive cells after adding EVs from mutated VSV-G engineered EVs for 48 hours in MSC-TL cells. VSV-G (K47Q) and VSV-G (P127D) were directly fused with Foldon-Intein-Cre and were used to produce the engineered EVs.

FIG. 21B depicts fluorescent microscopy images showing the GFP positive cells in MSC-TL cells after adding the indicated dose of EVs for 48 hours. Scale bar, 100 μm. FIG. 21B are representative confocal microscopy images demonstrating the GFP positive cells after adding the indicated doses of EVs for indicated time in Hela-TL cells. Scale bar, 100 μm. FIG. 21C is a western blot showing protein expression of directly fused mutated VSV-G-related constructs both in WCL of EV-producing cells and the isolated EVs evaluated by WB. Proteins from 5×10⁵ EV-producing cells and 1×10¹⁰ engineered vesicles were used for the assay. There were 2 bands when using Cre antibody for constructs with intein, with one band of un-cleaved full-length protein and the other cleaved free protein. There were 2 bands when using VSV-G antibody for constructs with both VSV-G and intein, with one band of un-cleaved full-length protein and the other VSV-G-Intein band. TSG101, syntenin-1 and β-actin were used as EV markers while Calnexin was used as cellular marker (endoplasmic reticulum) and should be absent for EV samples.

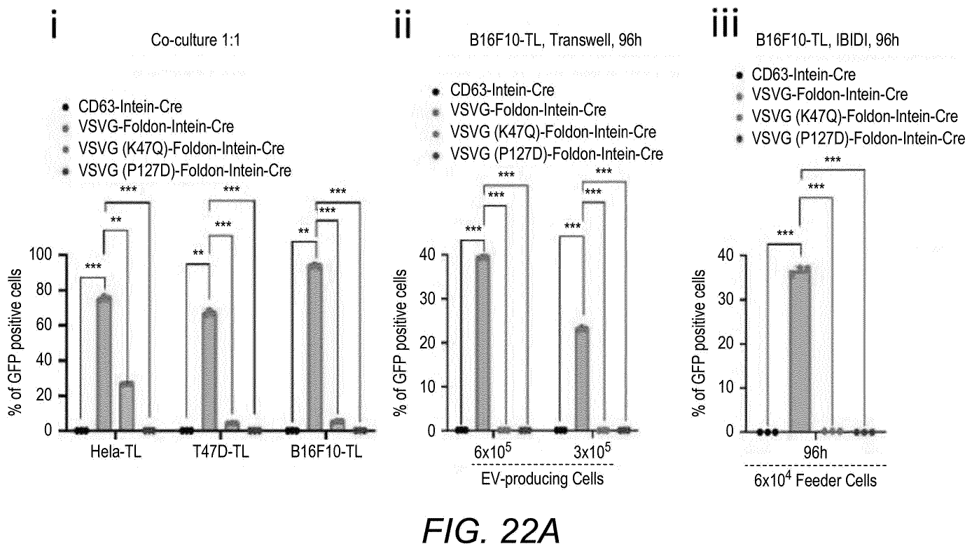

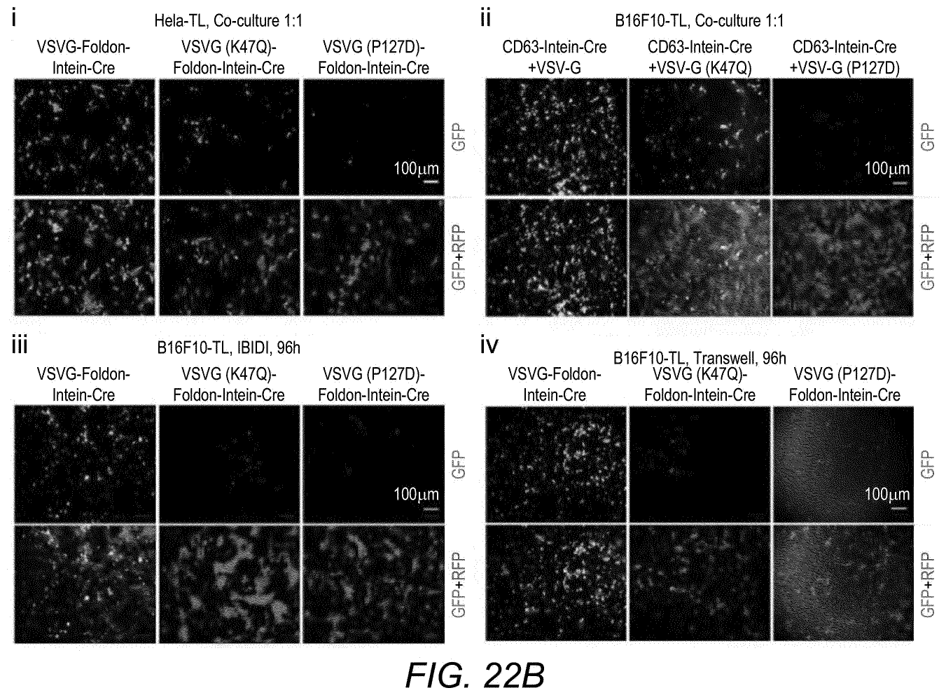

FIG. 22 is an evaluation of the function of VSV-G to enhance endosomal escape and the receptor-mediated endocytosis pathway for the entry of VSV-G engineered EVs by using P127D and K47D mutants for VFIC system (co-culture, IBIDI and transwell assays).

Referring specifically to FIG. 22A, there is shown a series of graphs depicting the results of a co-culture assay showing the percentage of GFP positive cells in Hela-TL, T47D-TL and B16F10-TL cells after 24 hours mixture of the EV-producing cells and reporter cells. P127D and K47D mutants were included to check the endosomal escape enhancer function of VSV-G and cell entry pathway of VSV-G engineered EVs respectively. Also shown is the percentage of GFP positive cells in B16F10-TL cells evaluated by transwell assay. The indicated numbers of EV-producing cells were seeded in the up chamber of the 24-well plate respectively and the reporter cells were added to the lower chamber, and the incubation time was 96 hours. Finally, FIG. 22A also shows the percentage of GFP positive cells in B16F10-TL cells evaluated by IBIDI assay. 6×10⁴ EV-producing HEK-293T cells and 4×10⁴ B16F10-TL cells were seeded into the surrounding reservoirs and central reservoir respectively and the assay was performed after 4 days' communication.

FIG. 22B shows representative images demonstrating GFP positive cells from co-culture assay in Hela-TL and B16F10-TL cells respectively. Scale bar, 100 μm.

Also shown are GFP positive cells in B16F10-TL cells as shown by fluorescent microscopy from IBIDI assay. The numbers of cells used were in line with (C). Scale bar, 100 μm.

FIG. 22B also shows GFP positive cells in B16F10-TL cells as shown by fluorescent microscopy from transwell assay.

### Example 20-Robust Gene Editing by Cas9/gRNA RNP and Meganuclease Targeting PCSK9 Using the VFIC and VEDIC Systems

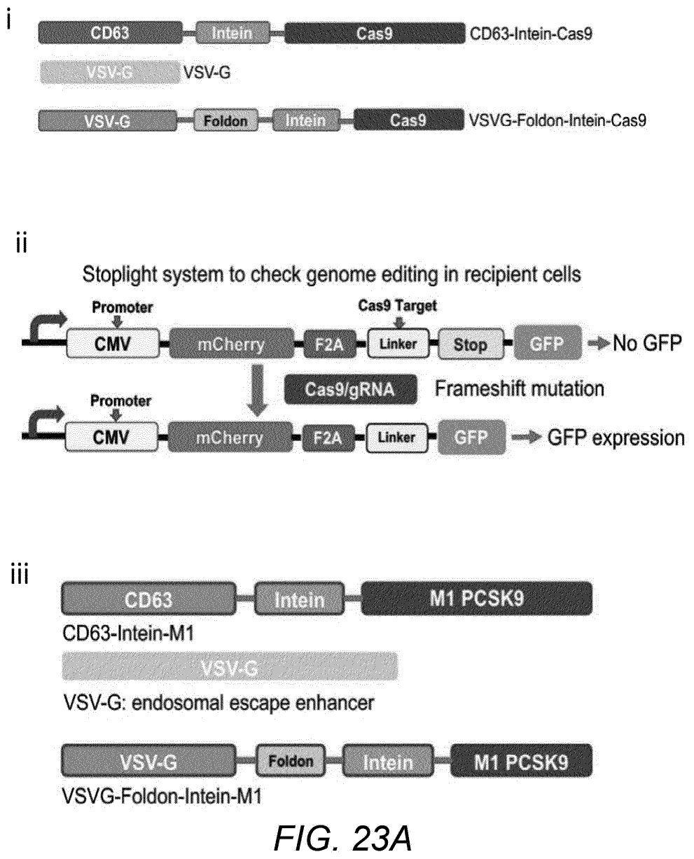

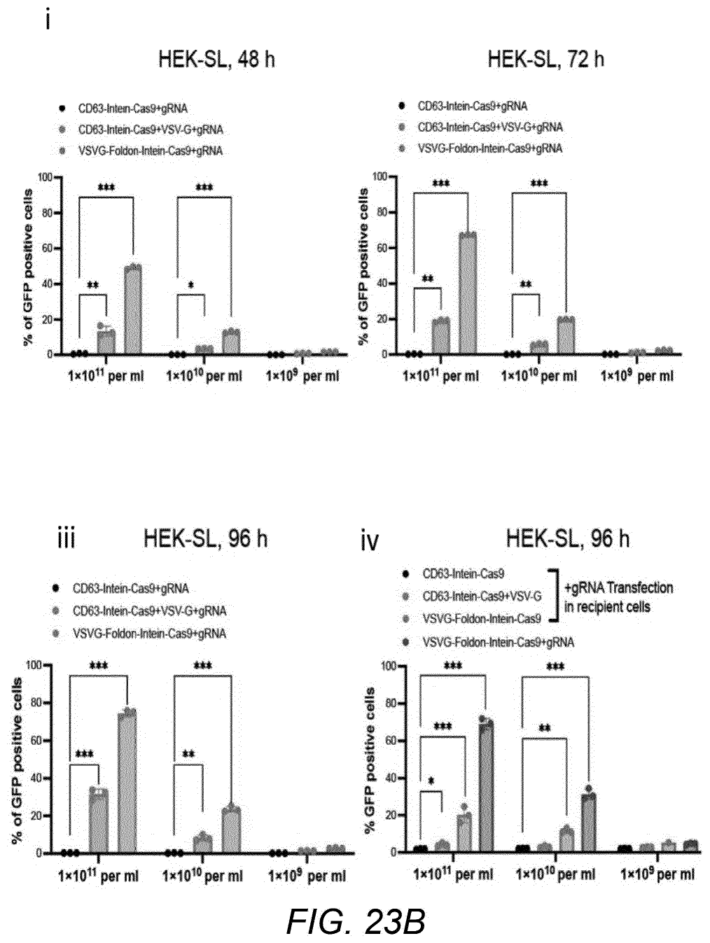

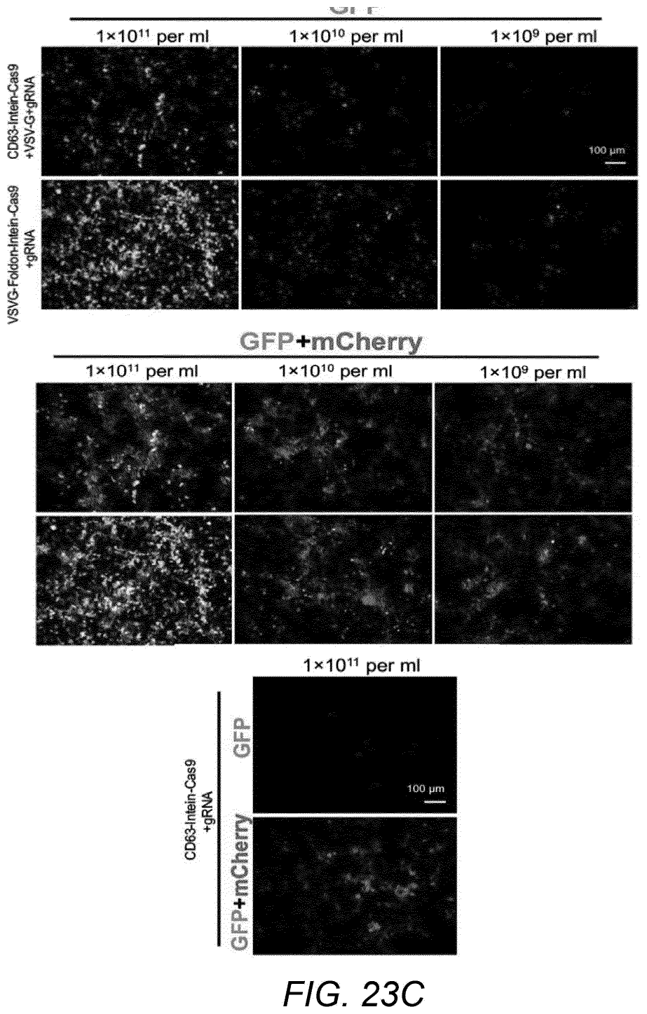

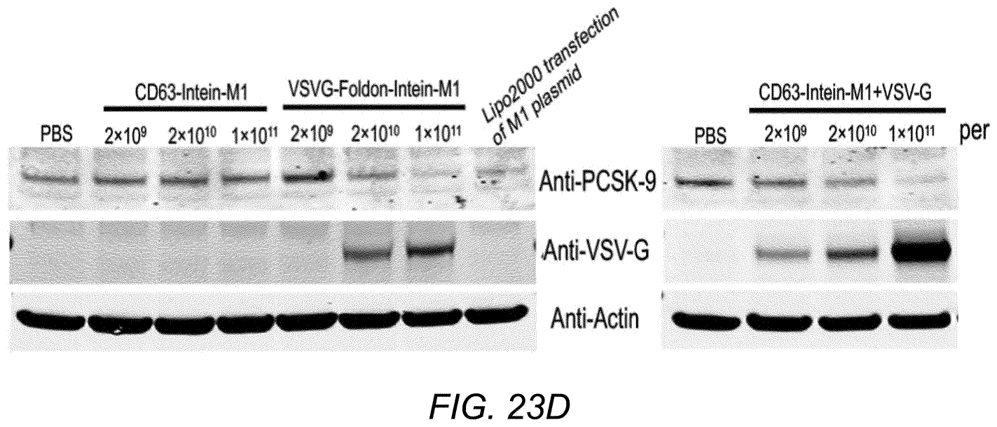

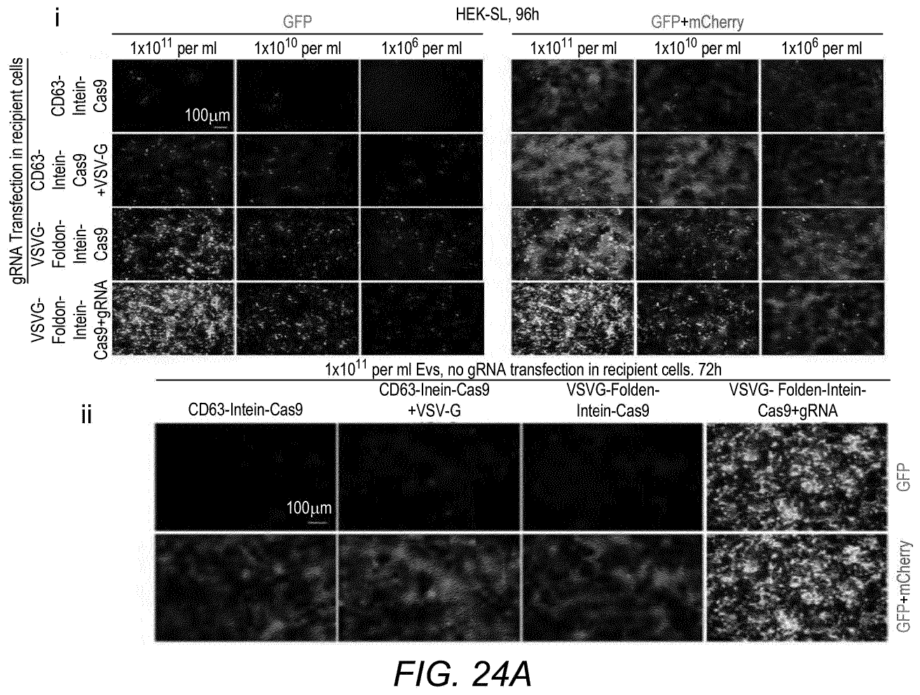

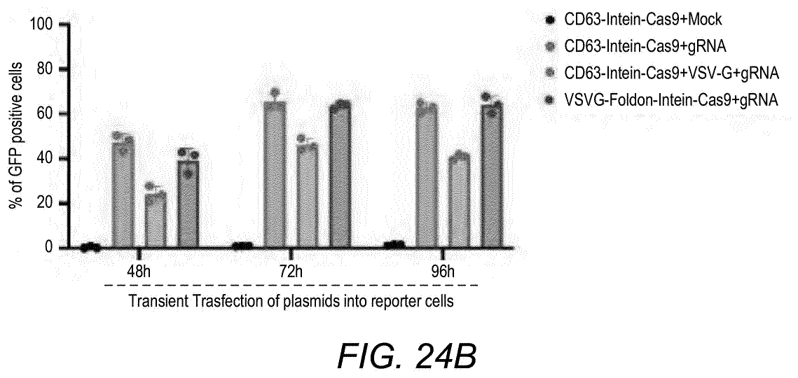

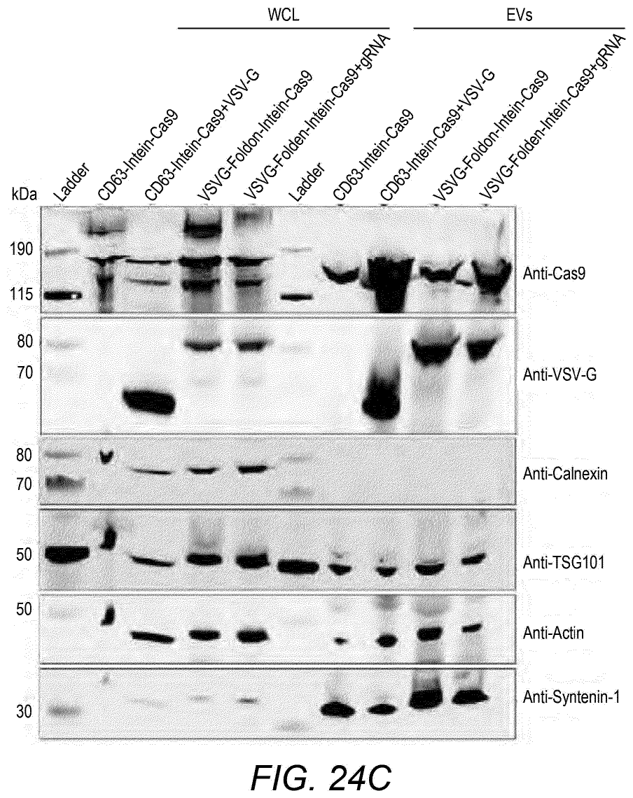

With reference to FIGS. 23 and 24, the inventors tested whether other cargos could be successfully delivered by the VEDIC and VFIC systems, Cre was replaced with Cas9 and generated CD63-Intein-Cas9 and VSVG-Foldon-Intein-Cas9.

FIG. 23A depicts the constructs and reporter cells used to generate and assay Cas9/gRNA RNP EVs. Also provided in FIG. 23A are the constructs generated for meganuclease targeting PCSK9 delivery.

FIG. 23B shows the percentage of GFP positive cells after adding engineered EVs, as assayed by flow cytometry after 48, 72 and 96 hours. Interestingly, the efficiency of VSVG-Foldon-Intein-Cas9+gRNA EVs was significantly higher than that of CD63-Intein-Cas9+VSV-G+gRNA EVs. Since it was hypothesized that the binding of Cas9 to the gRNA may be affected during EV biogenesis, Cas9 protein was separately delivered by engineered EVs and gRNA by transfection into reporter cells, but this actually resulted in less recombination.

FIG. 23C shows confocal immunofluorescence demonstrating gene-editing in recipient cells after treatment with different doses of engineered EVs. Scale bar=100 μm. Shown is the percentage of GFP positive cells when gRNA was transfected directly or delivered with EVs into reporter cells, as assayed by flow cytometry. Stoplight reporter cells with a linker and stop codon in between mCherry and GFP cassettes were used, such that successful editing resulted in GFP expression. Significant EV dose- and time-dependent genome editing was observed in cells treated with CD63-Intein-Cas9+VSV-G+gRNA and VSVG-Foldon-Intein-Cas9+gRNA EVs.

Finally, FIG. 23D shows a western blot evaluation of PCSK9 and VSV-G protein expression in Huh7 cells after treatment with different doses of EVs.

FIG. 24A is a representative image showing GFP positive cells after adding indicated dose of EVs for 72 hours. Scale bar, 100 μm. FIG. 24A also provides a representative image showing GFP positive cells after adding indicated dose of EVs for 96 hours in HEK-SL cells. GFP positive cells were measured 4 days later after adding EVs. Scale bar, 100 μm. For VSVG-Foldon-Intein-Cas9+gRNA group, close to 80% gene editing efficiency was achieved, which was higher than that demonstrated by directly transfecting Cas9 and gRNA plasmids into reporter cells.

FIG. 24B is a series of graphs showing the genome editing efficiency in HEK-SL reporter cells after transient transfection of the indicated constructs for indicated time.

FIG. 24C provides a western blot showing protein expression of Cas9 related constructs both in whole cell lysate (WCL) of EV-producing cells and the isolated EVs evaluated by western blot. Proteins from 5×10⁵ EV-producing cells and 1×10¹⁰ engineered vesicles were used for the assay. There were 2 bands when using Cas9 antibody for constructs with intein, with one band of un-cleaved full-length protein and the other cleaved free protein. TSG101, syntenin-1 and β-actin were used as EV markers while Calnexin was used as cellular marker (endoplasmic reticulum) and should be absent for EV samples.

Furthermore, constructs for the delivery of the previously described meganuclease targeting PCSK9 were generated and resulted in a significant decrease in PCSK9 expression after treating recipient cells with engineered EVs. Thus, the genome editing efficiency achieved by the VFIC system provided justification for utilizing these engineered EVs for in vivo studies.

### Example 21-Cre Mediated Recombination in Melanoma-Xenograft and Cre-LoxP Reporter tdTomato Mice by VEDIC and VFIC Systems after Local and Systemic Injections

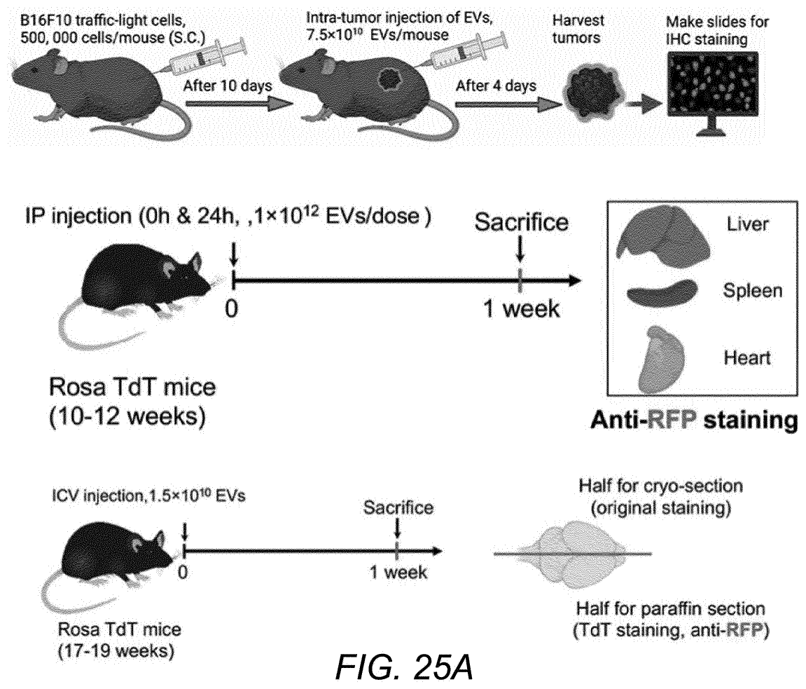

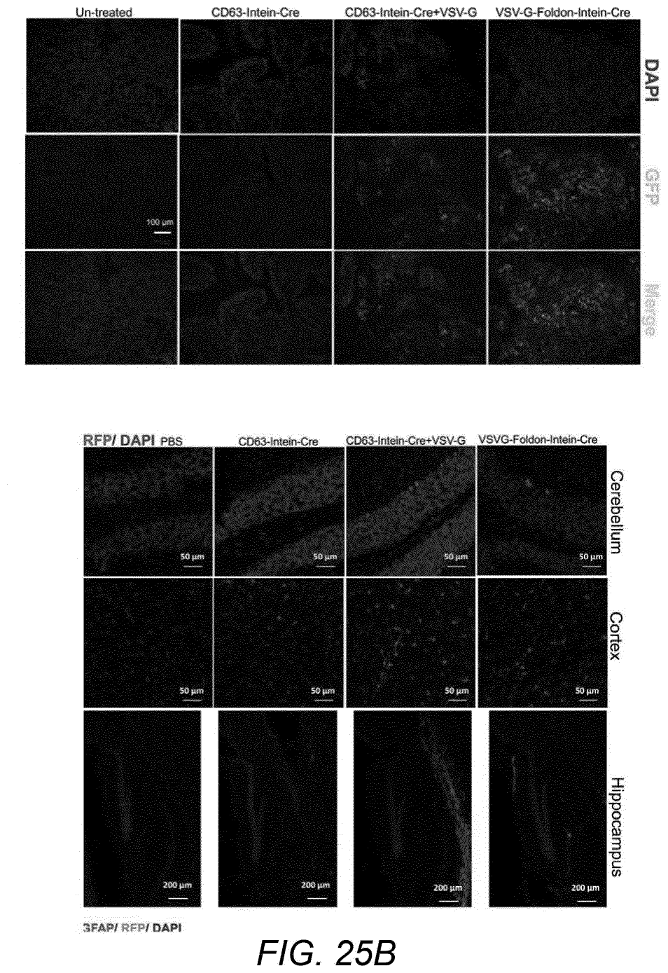

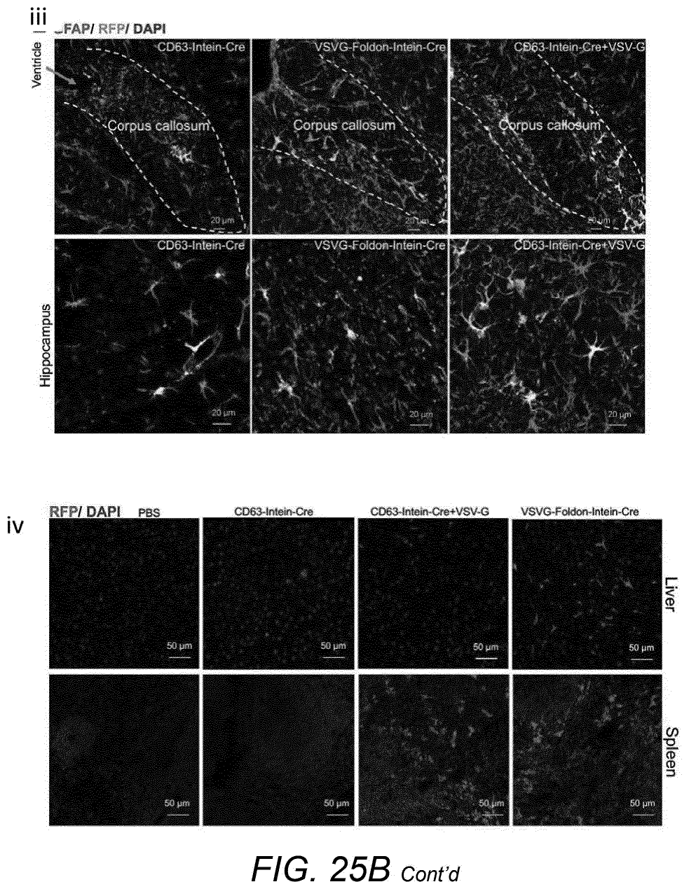

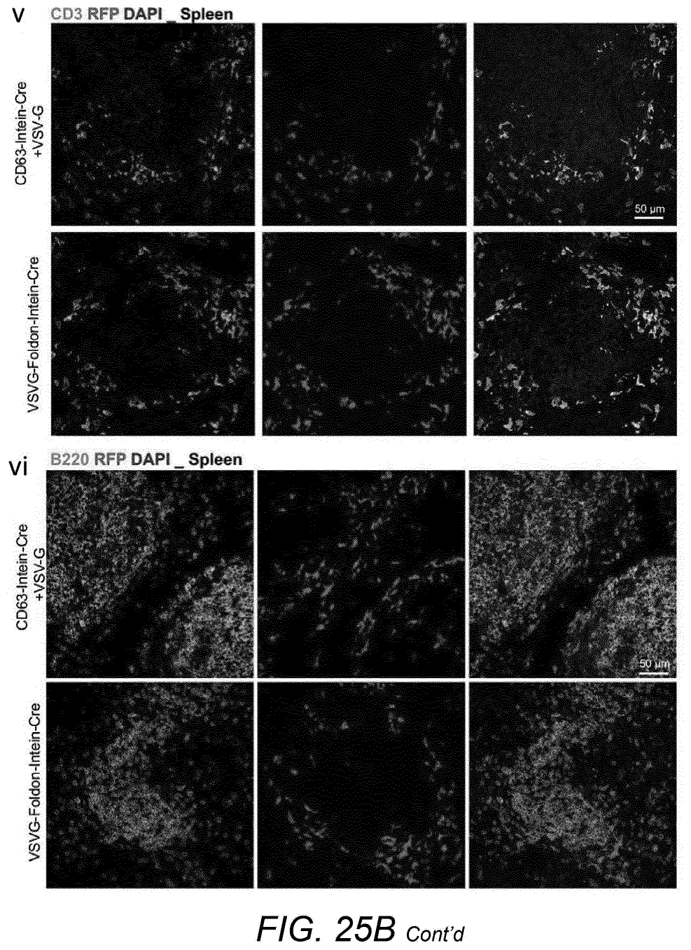

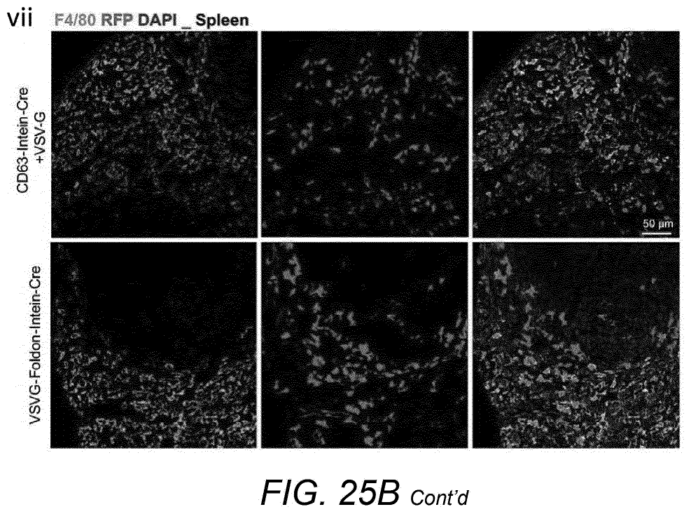

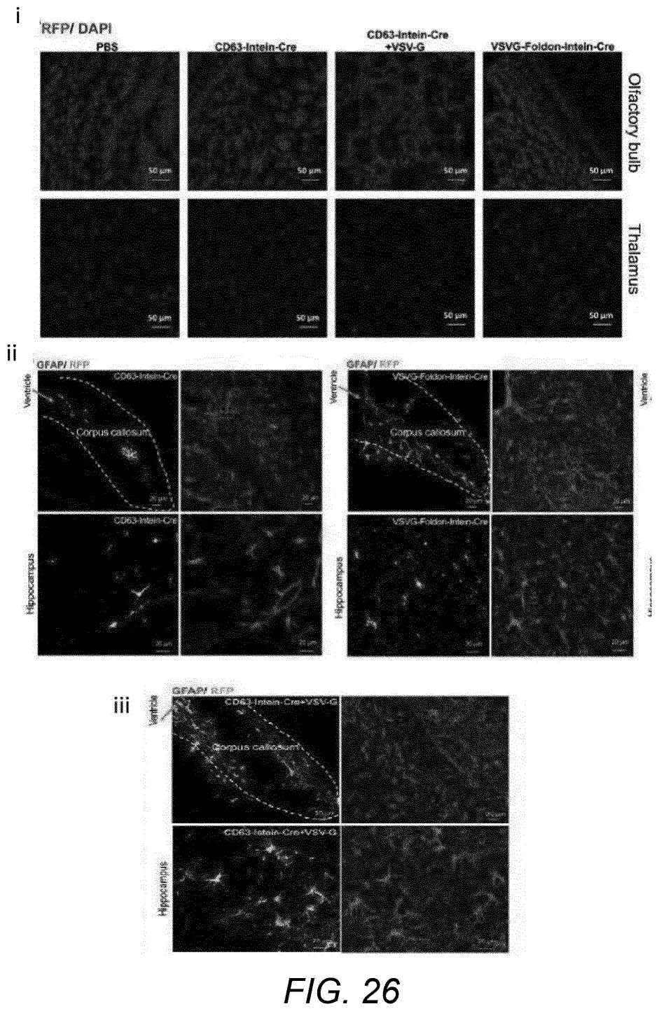

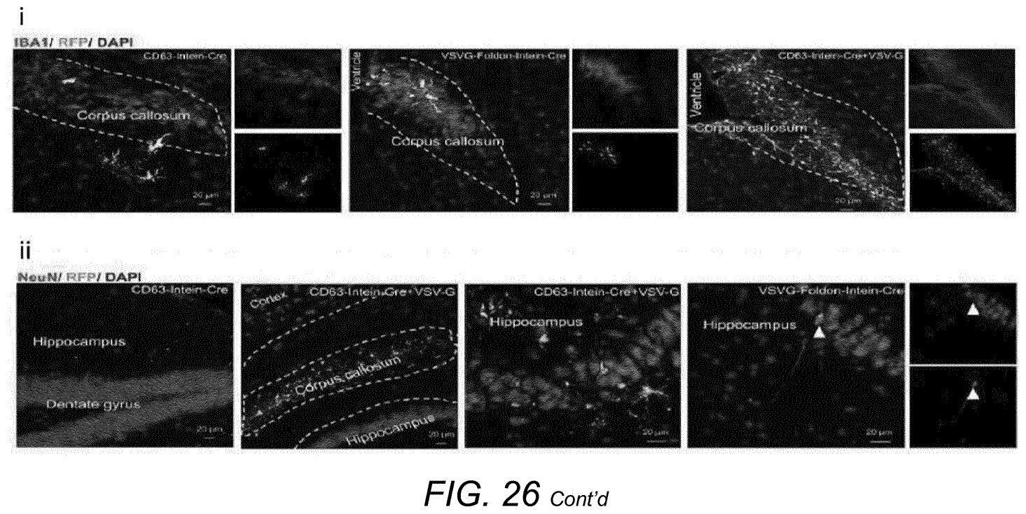

With reference to FIGS. 25 and 26 and based on the promising results achieved in examples 17 and 18, the applicability of VEDIC and VFIC engineered EVs in vivo was assessed further. Intratumoral injection of EVs was conducted in C57BL/6 mice bearing subcutaneous B16F10-TL melanoma xenografts.

As demonstrated in example 18, Cre recombination in melanoma-xenograft and Cre-LoxP reporter tdTomato mice is achieved by VEDIC and VFIC systems after local and systemic injections.

FIG. 25A provides the workflow for the intratumoral injection model and for the intracerebroventricular (ICV) injection of engineered EVs to deliver Cre in the brains of Cre-LoxP tdTomato reporter mice. FIG. 25A also provides a schematic workflow for the intraperitoneal (IP) injection of engineered EVs into Cre-LoxP tdTomato reporter mice.

Referring to FIG. 25B, there is provided a representative immunofluorescence image from tumor tissues after intratumoral injection of different engineered EVs to GFP expression. Scale bar=100 μm. N=3 mice per group. Also shown is RFP expression in different regions of brain after ICV injection of engineered EVs, as determined by immunofluorescence. Scale bar=50 μm for cerebellum and cortex and 200 μm for hippocampus. FIG. 25B also provides immunofluorescence images of depicted by co-staining of RFP with the astrocyte marker GFAP in corpus callosum (outlined by dashed lines) and hippocampus one week after ICV injection of engineered EVs. Scale bar=20 μm. Finally, FIG. 25B provides RFP expression in liver and spleen after IP injection of engineered EVs after one week. (Scale bar=50 μm).

Referring to FIG. 25B, 4 days after injection, tumors were harvested for immunohistochemistry, which showed significant GFP signals following treatment with VEDIC and VFIC EVs. Next, Cre-LoxP reporter tdTomato mice, whereby successful Cre delivery excises LoxP-stop signals and activates tdTomato, were used for intracerebroventricular (ICV) injection of the engineered EVs. One week after injection, the brains were harvested and stained for tdTomato (RFP) expression. Significant amounts of RFP expression were found in the cerebellum, cortex and hippocampus of VEDIC and VFIC, and to a lesser extent in CD63-Intein-Cre, injected animals.

To distinguish different populations of immune cells, the T-cell marker CD3, B-cell marker B220 and macrophage marker F4/80 were applied and revealed a high degree of RFP overlap with CD3 and F4/80, but low co-localization with B220. This is demonstrated in FIG. 25B, which shows co-staining of RFP with the T-cell marker CD3 in liver and spleen as detected by immunofluorescence one week after IP injection of engineered EVs. Scale bar=50 μm. Also shown is co-staining of RFP with the B cell marker B220 in liver and spleen one week after IP injection of engineered EVs. Scale bar=50 μm. Finally, co-staining of RFP and the macrophage marker F4/80 in liver and spleen one week after IP injection of engineered EVs is shown. Scale bar=50 μm. N=3 mice per group.

To further identify which types of brain cells internalized the engineered EVs, co-stained slides for both RFP and specific cell-marker genes were used as shown in FIG. 25B. The astrocyte marker GFAP co-localized with RFP in the corpus callosum and hippocampus in both VEDIC and VFIC treated animals showing co-staining of astrocyte marker GFAP with RFP in corpus callosum (highlighted by dashed lines) and hippocampus after I.C.V injection of different engineered EVs (separate images). Scale bar, 20 μm.). To a lesser extent, colocalization was also found in the CD63-Intein-Cre injected group.

With reference to FIG. 26, some RFP expression was observed in the olfactory bulb and thalamus of VEDIC and VFIC treated animals. RFP expression in olfactory bulb and thalamus after I.C.V injection of the engineered EVs for one week is evaluated by IHC staining. (Scale bar, 50 μm).

As demonstrated in FIG. 26, the microglia marker IBA1 also colocalized with RFP in the corpus callosum of VEDIC and VFIC treated mice, but not in the CD63-Intein-Cre group, demonstrated by co-staining of microglia marker IBA1 with RFP in corpus callosum (highlighted by dashed lines) after I.C.V injection of different engineered EVs. (Scale bar, 20 μm.).

In contrast, sporadic neurons internalized the engineered EVs only following VFIC injection, as evaluated by colocalization of RFP with the neuronal marker NeuN. This is further shown in FIG. 26, which shows Co-staining of neuron cell marker NeuN with RFP in different regions of brain after I.C.V injection of different engineered EVs. (Scale bar, 20 μm). White arrow indicated sporadic cells with colocalization between NeuN and RFP. N=3 mice per group. Based on the above results, it was concluded that astrocytes and microglia were the main cell types that internalized the engineered EVs in the brain.

After confirming Cre delivery by engineered EVs through local injections, the cell types targeted by systemic intraperitoneal (IP) injection were assessed. Thus, high-doses of engineered EVs were injected IP into Cre-LoxP reporter tdTomato mice and the liver, spleen and heart were then harvested for analysis by immunofluorescence one-week after injection. Substantial numbers of the cells in the liver and spleen were found to be RFP positive following VEDIC and VFIC, but not CD63-Intein-Cre, EV injection.

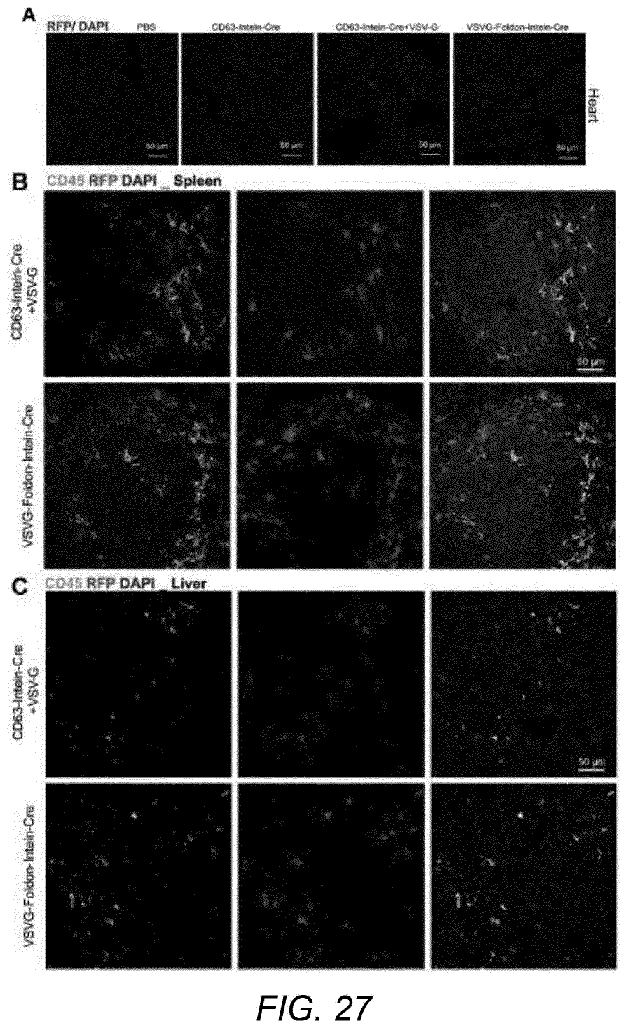

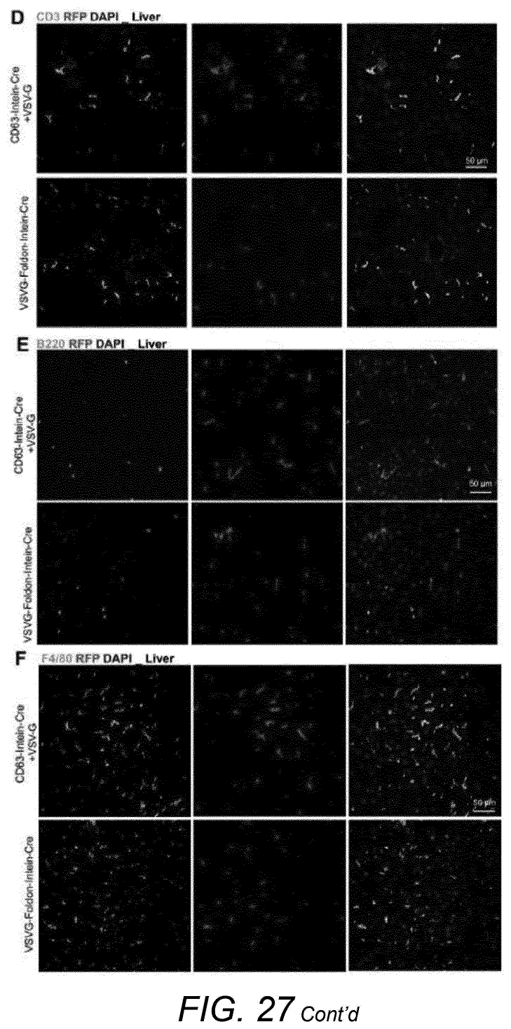

In contrast, there was no significant RFP expression in the heart as demonstrated in Figure. 27 and measured by IHC staining. (Scale bar, 50 μm). To further assess which cell types internalized the engineered EVs, co-staining of cell-specific markers with RFP was conducted. To begin with, liver and spleen tissue were stained with the general leukocyte marker CD45 and there was significant overlap with RFP. Also shown by FIG. 27 is T-cell marker CD3 colocalization with RFP in liver as detected by IHC co-staining after I.P injection of different engineered EVs for one week. Scale bar, 50 μm. B cell marker B220 was also shown to co-localize with RFP in liver after one week I.P injection of different engineered EVs. Scale bar, 50 μm. Finally shown is co-expression of macrophage marker F4/80 with RFP in liver evaluated by IHC co-staining after injecting engineered EVs for one week in Cre-LoxP reporter tdTomato mice. Scale bar, 50 μm. N=3 mice per group.

### Example 22-Treatment of LPS-Induced Sepsis by VEDIC and VFIC-Mediated Delivery of Super-Suppressor of NF-κB

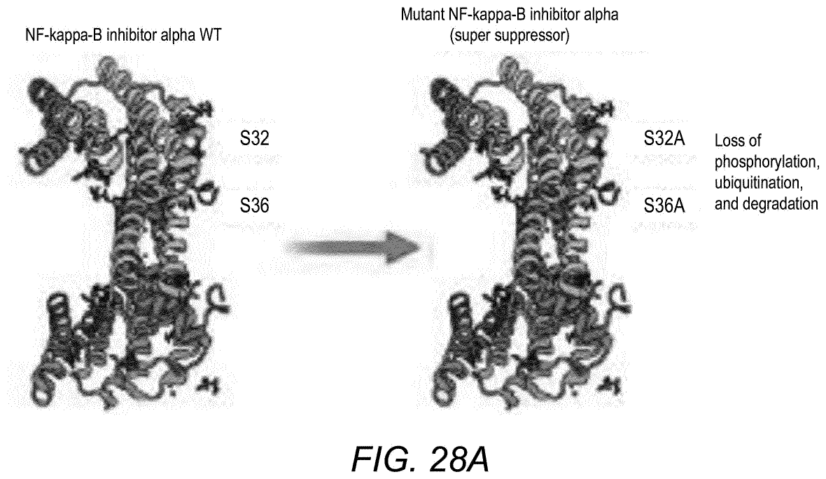

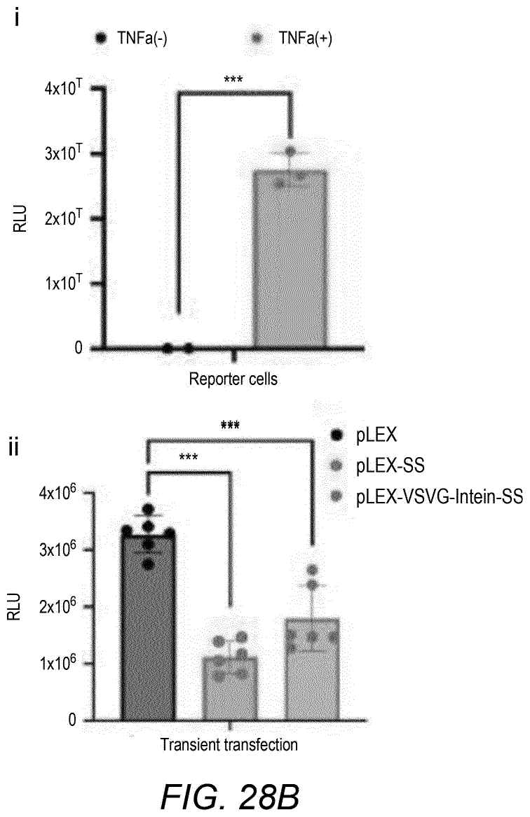

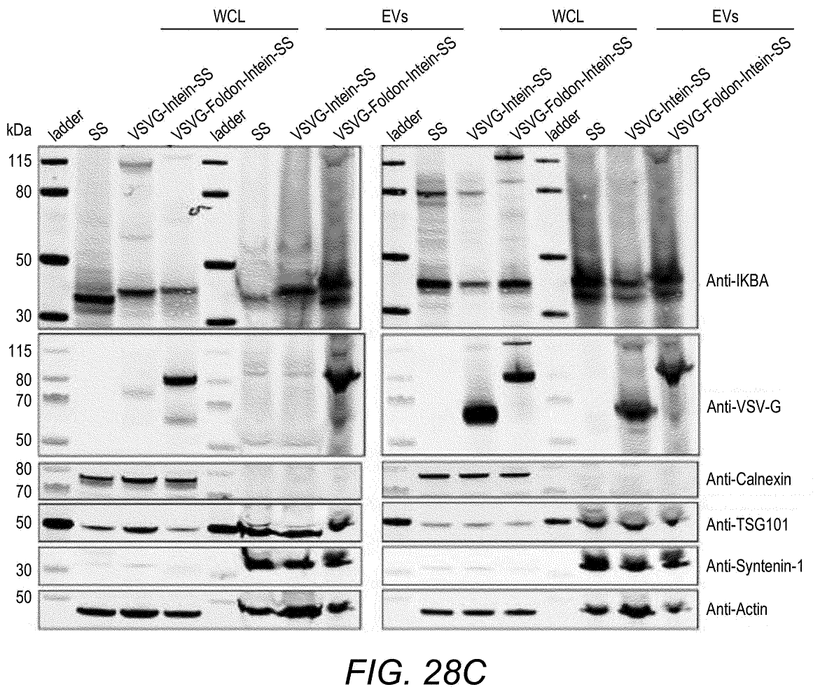

In order to demonstrate the applicability of the two systems in the treatment of disease, VEDIC and VFIC EVs were used to treat LPS-induced sepsis by delivering a previously reported super-suppressor of NF-κB activity (SS). This is shown in Figure. 28A, which shows the mutations and properties of super suppressor inhibitor of NF-κB.

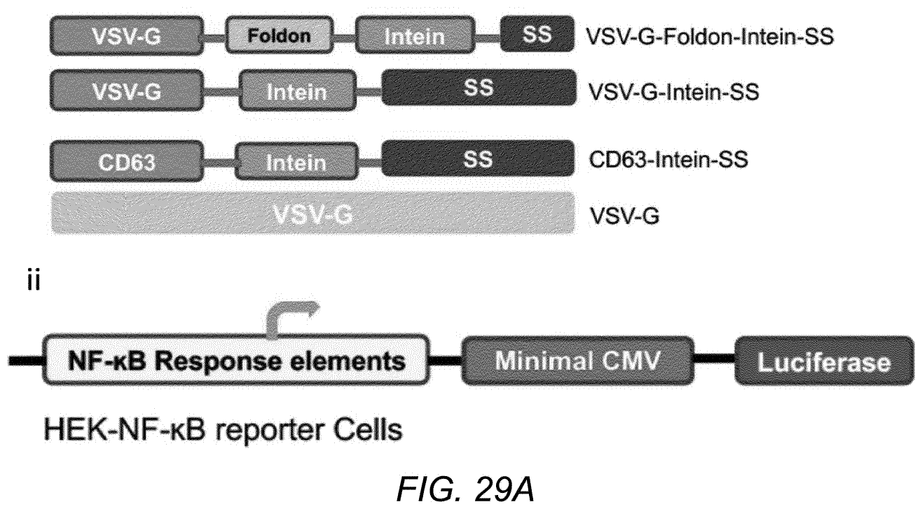

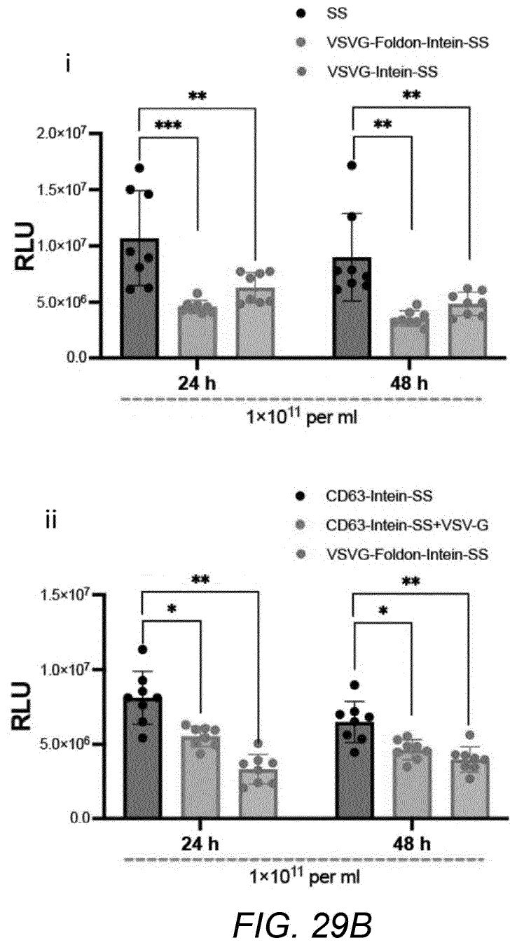

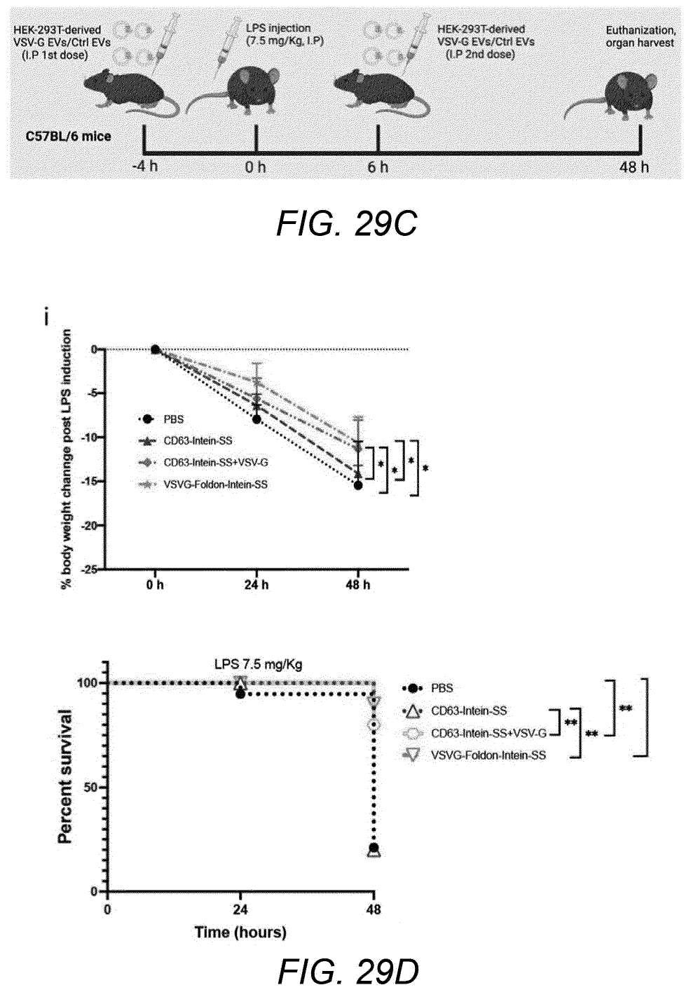

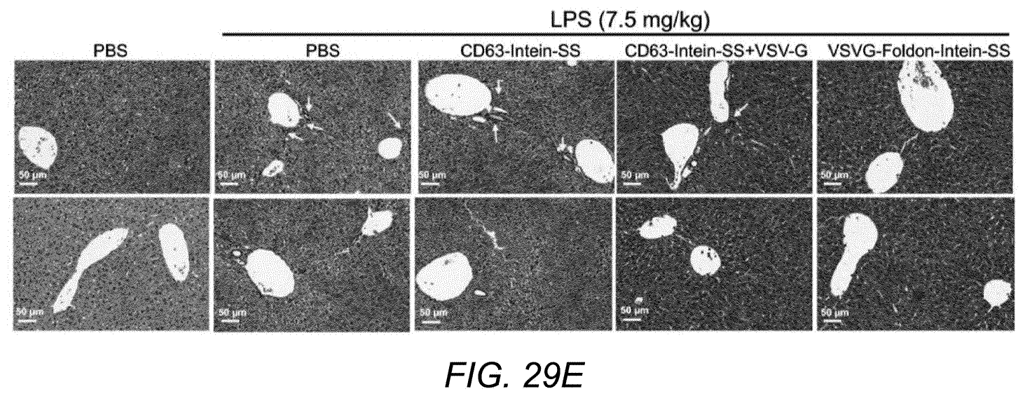

To accomplish this, CD63-Intein-SS, VSVG-Intein-SS and VSVG-Foldon-Intein-SS constructs were generated and HEK-NF-κB reporter cells were exploited to perform functional assays. The design of these constructs is shown in FIG. 29A.

Upon LPS stimulation in reporter cells, IκB is degraded, which allows NF-κB to translocate to the nucleus and drive downstream luciferase reporter gene expression. This is shown in FIG. 28B which demonstrate the expression of luciferases after 6 hours of TNF-α (10 ng/ml) stimulation in HEK-Blue-NF-κB reporter cells. There was a significant decrease of the luciferase signals from lysate of HEK-Blue-NF-κB reporter cells after transient transfection of SS-related constructs. The cells were transfected with the constructs for 48 hours first and then were stimulated by TNF-α for another 6 hours before luciferase detection.

As shown in FIG. 28C, protein expression of SS-related constructs both in whole cell lysate of EV-producing cells and the isolated EVs evaluated by western blot. Proteins from 5×105 EV-producing cells and 1×1010 engineered vesicles were used for the assay. There were 2 bands when using IKBA antibody for constructs with intein, with one band of un-cleaved full-length protein and the other cleaved free protein. There were 2 bands when using VSV-G antibody for constructs with both VSV-G and intein, with one band of un-cleaved full-length protein and the other VSV-G-Intein band. TSG101, syntenin-1 and β-actin were used as EV markers while Calnexin was used as cellular marker (endoplasmic reticulum) and should be absent for EV samples.

Using these cells, it was confirmed that the super supressor used here was able to block NF-κB reporter activation. Furthermore, VSVG-Foldon-Intein-SS, VSVG-Intein-SS and VSVG+CD63-Intein-SS delivered by EVs were better able to block reporter activation in vitro than un-engineered SS or CD63-Intein-SS alone.

This is demonstrated in FIG. 29B, which shows Luciferase activity from HEK-NF-κB reporter cells, 24 or 48 hours after treatment with engineered EVs, 6 hours after TNF-α stimulation. This provided the rationale for testing these EVs for in vivo experiments using an LPS-induced model of sepsis which is highlighted in FIG. 29C, which provides a schematic illustration of the workflow for the treatment of LPS-induced sepsis in mice by engineered EVs (5×10¹¹ EVs/mouse). Thus, engineered EVs were IP injected four hours before and six hours after injecting LPS to allow for and boost the binding of EV-delivered SS to NF-κB respectively. Subsequently, the weight and mortality of the mice was recorded at 24 and 48 hours after LPS treatment.

Compared to the PBS and CD63-Intein-SS injected groups, the body weight and survival of VSVG+CD63-Intein-SS and VSVG-Foldon-Intein-SS treated animals was significantly greater after 48 hours (FIG. 29D). This is shown in FIG. 29E, which are representative histology images (hematoxylin-eosin stain) of liver to show the aggregation of inflammatory cells (upper panel, yellow arrows indicate the aggregated inflammatory cells in portal areas) and the hydropic degeneration of hepatocytes (lower panel, red arrows indicate the hydropic degeneration of hepatocytes) after LPS induction. Scale bar=50 μm. These results demonstrated the adaptability of the VEDIC and VFIC systems for the treatment of a variety of diseases. The aggregation of inflammatory cells at the portal areas as well as the hydropic degeneration of hepatocytes were significant alleviated by treatment with VEDIC and VFIC EVs as demonstrated in FIG. 29E.

### Example 23-VEDIC and VFIC Systems Achieved an Unprecedented Level of Efficiency for the Intracellular Delivery of Functional and Therapeutic Proteins by Engineered EVs In Vitro and In Vivo

The data in the above examples provides solutions to the three major bottlenecks to EV-mediated intracellular protein delivery. The VEDIC and VFIC systems achieved an unprecedented level of efficiency for the intracellular delivery of functional and therapeutic proteins by engineered EVs in vitro and in vivo.

The examples show that functional cargo delivery by EVs requires the simultaneous application of strategies to enrich and liberate cargo inside EVs, as well as induce their release from endosomes in recipient cells. Many candidates were screened before selecting CD63 and VSV-G as the most potent mediator of these processes, respectively. The finding that VSV-G is functional in both of these steps resulted in the all-in-one VFIC system, which avoids the multi-plasmids co-transfection required by other technologies and led to more robust results.

Besides the robust intracellular protein delivery in reporter cells, these systems achieved dramatic functional protein delivery in mice by both local and systemic delivery. The cell types were profiled that internalized engineered EVs in the brain after ICV injection, and in spleen and liver after IP injection in Cre-LoxP reporter tdTomato mice. The data shows that astrocytes and microglia were the primary cell types targeted by EVs in the brain, while T-cells and macrophages internalized the majority of engineered EVs in spleen and liver. This study shows the functional delivery of protein cargo by EVs injected in vivo. In order to better understand the endosomal escape mediating effects of VSV-G, two mutants (K47Q and P127D) of VSV-G were used. The abilities of VSV-G to act during both endocytosis and membrane fusion were important in inducing the diffuse distribution of GFP, this was indicative of functional protein delivery. This clearly demonstrates endosomal escape of engineered EV cargo proteins in recipient cells and provides a convenient assay to explore this process further. This data also suggested that receptor-mediated endocytosis is the primary pathway responsible for the entry of VSV-G engineered EVs into recipient cells.

The disclosure is not limited to the embodiments and examples hereinbefore described which may be varied in both construction and detail.

### SEQUENCE LISTING

### Polynucleotide Sequences

SEQ ID No 1: VSV-G
ATGAAGTGTCTCCTTTATCTCGCCTTTCTCTTTATAGGGGTCAACTGCAA
ATTTACGATCGTTTTTCCGCACAATCAGAAAGGAAACTGGAAAAATGTGC
CATCTAACTACCATTACTGCCCTTCTAGCTCTGACCTTAACTGGCATAAT
GATTTGATCGGGACAGCGCTCCAAGTCAAAATGCCGAAATCCCACAAG
GCGATCCAAGCGGATGGCTGGATGTGTCATGCGTCCAAATGGGTTACA
ACTTGCGATTTCCGGTGGTACGGGCCCAAATACATCACCCACAGTATTA
GAAGCTTTACACCTAGTGTAGAACAGTGCAAAGAGAGCATCGAACAAAC
CAAACAGGGCACTTGGCTCAATCCAGGCTTTCCCCCGCAGTCTTGCGG
CTACGCCACTGTGACCGATGCGGAGGCCGTGATCGTCCAAGTCACCCC
CCATCACGTCCTGGTTGACGAATATACAGGGGAGTGGGTAGATAGTCAA
TTTATCAACGGTAAATGCAGTAATTATATATGCCCCACCGTGCATAATTC
TACCACATGGCACAGCGATTACAAGGTGAAAGGGTTGTGCGATTCAAAC
CTCATATCAATGGACATCACCTTCTTTTCAGAAGATGGTGAGCTCAGTTC
TCTTGGGAAGGAAGGTACTGGGTTTCGGAGTAATTACTTCGCCTACGAA
ACGGGTGGCAAGGCCTGCAAGATGCAGTACTGCAAACACTGGGGGGTC
CGCCTGCCAAGTGGGGTTTGGTTCGAAATGGCGGACAAGGATTTGTTT
GCGGCGGCGCGGTTCCCAGAGTGTCCAGAAGGGTCATCCATATCTGCG
CCTTCTCAAACGTCTGTGGACGTGTCTCTTATCCAAGACGTGGAGAGGA
TTTTGGATTACTCACTTTGCCAAGAAACGTGGAGTAAGATAAGAGCGGG
TCTGCCAATAAGTCCGGTGGATCTTAGTTACCTGGCACCAAAAAACCCA
GGCACCGGGCCCGCCTTCACTATCATAAACGGGACTCTGAAATATTTTG
AGACGAGATATATCCGGGTTGATATCGCTGCGCCGATACTCTCCAGGAT
GGTCGGCATGATTAGTGGTACGACGACTGAGCGAGAACTGTGGGATGA
CTGGGCTCCGTACGAAGACGTTGAGATCGGTCCAAACGGGGTCTTGCG
CACATCTAGTGGTTATAAGTTCCCGCTGTATATGATTGGTCACGGTATG
CTTGACTCAGACCTCCATCTCAGTTCCAAGGCTCAAGTGTTTGAGCACC
CACACATCCAAGATGCGGCAAGTCAACTTCCTGACGATGAGTCACTGTT
CTTTGGGGACACTGGACTCTCCAAGAATCCAATTGAGTTGGTTGAAGGA
TGGTTTTCATCATGGAAAAGTAGTATAGCCTCTTTTTTCTTCATAATTGGA
CTTATCATAGGTTTGTTCTTGGTACTCAGAGTCGGTATACACCTGTGTAT
AAAATTGAAGCACACTAAGAAAAGGCAAATCTATACGGACATAGAAATG
AATCGATTGGGGAAG
SEQ ID No 2: CVG
ATGAATTTTCTCCTTCTGACATTTATCGTACTGCCACTGTGTTCTCATGC
TAAGTTCAGTATCGTGTTCCCCCAGAGTCAGAAGGGTAATTGGAAAAAT
GTGCCCTCCTCATACCACTACTGCCCCAGTTCATCAGACCAGAACTGGC
ATAACGACCTGCTTGGGATCACAATGAAGGTTAAAATGCCCAAAACACA
CAAGGCTATCCAGGCCGATGGCTGGATGTGCCATGCTGCCAAGTGGAT
CACCACCTGCGATTTTCGCTGGTACGGGCCAAAATACATCACTCATTCA
ATTCACTCAATACAGCCCACCTCTGAACAGTGCAAGGAGTCTATTAAGC
AAACCAAGCAGGGCACATGGATGAGCCCCGGCTTTCCACCTCAGAATT
GCGGGTACGCAACAGTGACCGACTCTGTGGCAGTCGTTGTGCAAGCGA
CCCCCCATCATGTCCTGGTGGACGAATACACCGGCGAGTGGATAGATT
CTCAGTTCCCAAATGGCAAATGCGAGACTGAAGAGTGTGAGACAGTGC
ATAACTCAACAGTGTGGTACAGCGACTATAAGGTAACCGGTTTGTGTGA
CGCAACCCTGGTGGATACCGAAATCACGTTCTTCTCTGAAGACGGAAAG
AAGGAATCCATCGGCAAGCCCAACACAGGTTACCGTTCCAACTACTTCG
CATATGAAAAGGGCGATAAAGTATGTAAGATGAACTACTGTAAGCATGC
AGGCGTTCGACTTCCTAGTGGCGTGTGGTTCGAATTTGTGGATCAGGAT
GTTTATGCTGCCGCCAAGTTACCTGAATGCCCCGTGGGCGCCACTATTA
GTGCCCCAACACAGACATCCGTGGATGTGTCATTAATCCTTGACGTGGA
AAGAATCCTGGACTACTCACTCTGTCAGGAGACCTGGTCTAAGATCCGG
AGCAAGCAGCCTGTAAGTCCCGTAGACCTCTCTTATCTGGCCCCAAAAA
ACCCGGGCACAGGGCCTGCCTTCACCATAATCAACGGCACACTGAAGT
ACTTCGAGACTCGCTATATTAGGATTGACATCGACAACCCAATAATCTCA
AAAATGGTGGGGAAGATTAGCGGATCACAGACAGAGAGGGAGCTGTGG
ACAGAGTGGTTTCCTTACGAAGGCGTAGAAATTGGCCCGAACGGAATC
CTCAAGACCCCTACTGGATATAAGTTTCCTCTGTTTATGATCGGACACG
GCATGCTTGACAGCGACCTGCATAAGACCTCTCAGGCCGAGGTCTTCG
AGCACCCCCACCTGGCGGAGGCTCCCAAGCAGCTGCCAGAGGAAGAG
ACATTGTTCTTCGGCGACACAGGGATAAGCAAAAACCCTGTCGAACTGA
TTGAGGGCTGGTTCAGCTCATGGAAATCAACCGTCGTGACATTCTTCTT
CGCAATAGGTGTGTTCATACTGCTCTATGTCGTCGCTCGGATTGTTATC
GCTGTTAGATACCGCTACCAAGGCTCAAACAATAAGAGAATCTACAACG
ACATCGAGATGAGCAGGTTTAGAAAGGTGAGCGGCTGGCGGCTGTTCA
AGAAGATTAGC
SEQ ID No 3: PFV
ATGGCCCCTCCCATGACCCTGCAGCAGTGGATCATCTGGAAGAAGATG
AACAAGGCCCACGAGGCCCTGCAGAACACCACCACCGTGACCGAGCA
GCAGAAAGAGCAGATCATCCTGGACATCCAGAACGAGGAAGTGCAGCC
CACCAGGCGGGACAAGTTCAGATACCTGCTGTACACCTGCTGCGCCAC
CTCCAGCCGGGTGCTGGCCTGGATGTTCCTGGTGTGCATCCTGCTGAT
CATCGTGCTGGTGTCCTGCTTCGTGACCATCAGCCGGATCCAGTGGAA
CAAGGACATCCAGGTGCTGGGCCCCGTGATCGACTGGAACGTGACCCA
GCGGGCCGTGTACCAGCCCCTGCAGACCCGGOGGATCGCCCGGTCCC
TGCGGATGCAGCACCCCGTGCCCAAGTACGTGGAGGTGAACATGACCA
GCATCCCCCAGGGCGTGTACTACGAGCCCCACCCCGAGCCCATCGTG
GTGAAAGAAAGAGTGCTGGGCCTGAGCCAGATCCTGATGATCAACAGC
GAGAACATCGCCAACAACGCCAACCTGACCCAGGAAGTGAAGAAACTG
CTGACCGAGATGGTGAACGAAGAGATGCAGAGCCTGAGCGACGTGATG
ATCGACTTCGAGATCCCCCTGGGCGACCCCAGGGACCAGGAACAGTAC
ATCCACCGGAAGTGCTACCAGGAATTTGCCAACTGCTACCTGGTGAAGT
ACAAAGAGCCCAAGCCCTGGCCCAAAGAGGGCCTGATCGCCGACCAGT
GCCCCCTGCCCGGCTATCACGCCGGCCTGACCTACAACCGGCAGAGC
ATCTGGGACTACTACATCAAGGTGGAGAGCATCAGGCCCGCCAACTGG
ACCACCAAGAGCAAGTACGGCCAGGCCCGGCTGGGCAGCTTCTACATC
CCCAGCAGCCTGCGGCAGATCAACGTGAGCCACGTGCTGTTCTGCAGC
GACCAGCTGTACAGCAAGTGGTACAACATCGAGAACACCATCGAGCAG
AACGAGCGGTTCCTGCTGAACAAGCTGAATAACCTGACCAGCGGCACC
AGCGTGCTGAAGAAGAGAGCCCTGCCCAAGGACTGGTCCAGCCAGGG
CAAGAACGCCCTGTTCCGGGAGATCAATGTGCTGGACATCTGCAGCAA
GCCCGAGAGCGTGATCCTGCTGAATACCAGCTACTACAGCTTCAGCCT
GTGGGAGGGCGACTGCAACTTCACCAAGGACATGATCAGCCAGCTGGT
GCCCGAGTGCGACGGCTTCTACAACAACTCCAAGTGGATGCACATGCA
CCCCTACGCCTGCCGGTTCTGGCGGAGCAAGAACGAGAAAGAGGAAAC
CAAGTGCCGGGACGGCGAGACCAAGCGGTGCCTGTACTACCCCCTGT
GGGACAGCCCTGAGAGCACCTACGACTTCGGCTACCTGGCCTACCAGA
AGAACTTCCCCAGCCCCATCTGCATCGAACAGCAGAAGATCCGGGACC
AGGACTACGAGGTGTACAGCCTGTACCAGGAATGCAAGATCGCCAGCA
AGGCCTACGGCATCGACACCGTGCTGTTCAGCCTGAAGAATTTCCTGAA
CTACACCGGCACCCCCGTGAACGAGATGCCCAACGCCAGGGCCTTCGT
GGGCCTGATTGACCCCAAGTTCCCCCCCAGCTACCCCAACGTGACCCG
GGAGCACTACACCAGCTGCAACAACCGGAAGCGGCGGAGCGTGGACA
ACAACTACGCCAAGCTGCGGAGCATGGGCTACGCTCTGACAGGCGCCG
TGCAGACCCTGTCCCAGATCAGCGACATCAACGACGAGAACCTGCAGC
AGGGCATCTACCTGCTGCGGGACCACGTGATCACCCTGATGGAAGCCA
CCCTGCACGACATCAGCGTGATGGAAGGCATGTTCGCCGTGCAGCACC
TGCACACCCACCTGAATCACCTGAAAACCATGCTGCTGGAACGGCGCA
TCGACTGGACCTACATGAGCAGCACCTGGCTGCAGCAGCAGCTGCAGA
AAAGCGACGACGAGATGAAGGTGATCAAGCGGATCGCCAGATCTCTGG
TGTACTACGTGAAGCAGACCCACAGCAGCCCCACCGCCACCGCCTGGG
AGATCGGCCTGTACTATGAGCTGGTGATCCCCAAGCACATCTACCTGAA
CAACTGGAATGTGGTGAACATCGGCCACCTGGTGAAAAGCGCCGGACA
GCTGACCCACGTGACCATCGCCCACCCCTACGAGATCATCAACAAAGA
ATGCGTGGAGACCATCTATCTGCACCTGGAAGATTGCACCCGGCAGGA
CTACGTGATCTGCGACGTGGTGAAGATCGTGCAGCCCTGCGGCAACAG
CAGCGACACCAGCGACTGCCCCGTGTGGGCCGAGGCCGTGAAAGAAC
CCTTCGTGCAGGTGAACCCCCTGAAGAACGGCTCCTACCTGGTGCTGG
CCAGCAGCACCGACTGCCAGATCCCCCCCTACGTGCCCAGCATCGTGA
CCGTGAATGAGACCACCTCCTGCTTCGGCCTGGACTTCAAGCGGCCCC
TGGTGGCCGAGGAAAGACTGAGCTTCGAGCCCCGGCTGCCCAACCTG
CAGCTGAGGCTGCCCCACCTGGTGGGCATCATCGCCAAGATCAAGGGC
ATCAAGATCGAGGTGACCAGCAGCGGCGAGAGCATCAAAGAACAGATC
GAGCGGGCCAAGGCCGAGCTGCTGCGGCTGGATATCCACGAGGGCGA
CACACCCGCCTGGATCCAGCAGCTGGCCGCCGCCACCAAGGACGTGT
GGCCCGCTGCAGCCAGCGCCCTGCAGGGCATCGGCAACTTTCTGAGC
GGCACCGCCCAGGGCATCTTCGGCACCGCCTTCTCCCTGCTGGGCTAC
CTGAAGCCCATCCTGATCGGCGTGGGCGTGATTCTGCTGGTGATTCTG
ATCTTCAAGATCGTGAGCTGGATCCCCACCAAGAAAAAGAACCAG
SEQ ID No 4: CD63
ATGGCTGTGGAAGGCGGAATGAAGTGCGTGAAGTTCCTGCTGTACGTG
CTGCTGCTGGCCTTCTGTGCTTGTGCCGTGGGACTGATTGCCGTTGGA
GTTGGAGCACAGCTGGTGCTGAGCCAGACAATCATCCAGGGCGCCACA
CCTGGATCTCTGCTGCCCGTGGTTATTATCGCCGTGGGCGTGTTCCTGT
TCCTGGTGGCCTTTGTGGGATGTTGCGGCGCCTGCAAAGAAAACTACT
GCCTGATGATTACCTTCGCCATCTTCCTGAGCCTGATCATGCTGGTGGA
AGTGGCCGCTGCCATTGCCGGCTACGTGTTCAGAGACAAAGTGATGAG
CGAGTTCAACAACAACTTCCGGCAGCAGATGGAAAACTACCCCAAGAAC
AACCACACCGCCAGCATCCTGGACAGAATGCAGGCCGACTTCAAGTGT
TGCGGAGCCGCCAACTACACCGACTGGGAGAAGATCCCCAGCATGAGC
AAGAACAGAGTGCCCGACAGCTGCTGCATCAACGTGACAGTTGGCTGC
GGCATCAATTTCAACGAGAAGGCCATCCACAAAGAGGGCTGCGTCGAG
AAGATCGGCGGCTGGCTGAGAAAGAACGTGCTGGTTGTTGCCGCCGCT
GCTCTGGGAATTGCCTTCGTGGAAGTGCTGGGCATCGTGTTCGCCTGC
TGTCTGGTCAAGAGCATCCGCAGCGGCTACGAAGTGATG
SEQ ID No 5: CD9
ATGCCTGTCAAGGGCGGTACAAAATGCATCAAGTACCTTTTGTTTGGTTT
TAATTTCATCTTCTGGCTTGCAGGCATAGCTGTGCTGGCCATCGGGCTT
TGGCTGCGGTTTGATAGCCAAACAAAGAGTATCTTTGAGCAGGAAACTA
ATAATAACAATAGTAGTTTTTATACTGGCGTCTATATTCTTATTGGTGCAG
GAGCCCTCATGATGCTTGTCGGCTTTCTGGGGTGTTGTGGGGCCGTTC
AGGAATCCCAGTGTATGCTTGGACTTTTCTTTGGGTTCCTGCTCGTTATC
TTCGCCATCGAGATCGCAGCGGCGATATGGGGTTACTCACATAAGGAC
GAAGTAATCAAGGAAGTTCAGGAGTTCTATAAAGACACGTATAATAAACT
GAAAACCAAAGATGAGCCCCAACGGGAGACCCTTAAAGCGATCCACTA
CGCTCTCAACTGCTGCGGACTTGCAGGTGGCGTTGAACAATTCATATCC
GACATCTGCCCGAAAAAGGATGTACTCGAAACTTTTACTGTCAAGTCAT
GTCCGGACGCTATAAAGGAAGTGTTCGATAATAAGTTTCACATAATCGG
CGCAGTTGGGATAGGAATAGCCGTAGTGATGATATTTGGCATGATTTTT
AGTATGATATTGTGTTGTGCCATAAGGAGGAATAGGGAGATGGTA
SEQ ID No 6: CD81
ATGGGTGTTGAAGGGTGTACGAAGTGTATTAAATATCTTTTGTTTGTATT
CAACTTCGTATTTTGGCTTGCTGGCGGAGTAATTCTGGGGGTAGCACTG
TGGCTGCGGCACGACCCCCAAACGACTAACTTGCTTTATCTGGAATTGG
GCGACAAACCGGCGCCCAACACGTTCTACGTCGGTATTTATATCCTTAT
CGCAGTAGGGGCTGTTATGATGTTCGTAGGTTTTCTGGGGTGTTACGGC
GCAATACAGGAGTCACAGTGTCTGCTCGGTACGTTTTTTACTTGTCTCG
TAATTCTTTTCGCCTGCGAAGTCGCGGCAGGTATCTGGGGTTTTGTAAA
CAAAGATCAAATAGCAAAGGACGTAAAACAATTTTATGACCAAGCTCTGC
AGCAGGCGGTTGTAGACGATGACGCGAACAACGCTAAAGCCGTTGTCA
AGACTTTCCATGAAACACTCGACTGTTGCGGCTCCAGTACACTTACCGC
ACTGACCACGTCAGTGCTGAAAAACAACCTGTGCCCCAGTGGCTCAAAC
ATCATATCTAACTTGTTCAAGGAAGATTGCCACCAAAAGATTGACGATCT
GTTTTCAGGCAAATTGTATCTCATTGGGATTGCCGCGATCGTTGTCGCA
GTGATTATGATCTTTGAGATGATCCTGAGCATGGTACTTTGCTGTGGTAT
AAGAAATTCTTCTGTTTAT
SEQ ID No 7: PTGFRN
atggggcgcctggcctcgaggccgctgctgctggcgctcctgtcgttggctctttgccgagggcgtgtggtg
agagtccccacagcgaccctggttcgagtggtgggcactgagctggtcatcccctgcaacgtcagtgact
atgatggccccagcgagcaaaactttgactggagcttctcatctttggggagcagctttgtggagcttgcaa
gcacctgggaggtggggttcccagcccagctgtaccaggagcggctgcagaggggcgagatcctgtta
aggcggactgccaacgacgccgtggagctccacataaagaacgtccagccttcagaccaaggccact
acaaatgttcaacccccagcacagatgccactgtccagggaaactatgaggacacagtgcaggttaaa
gtgctggccgactccctgcacgtgggccccagcgcgcggcccccgccgagcctgagcctgcgggagg
gggagcccttcgagctgcgctgcaccgccgcctccgcctcgccgctgcacacgcacctggcgctgctgtg
ggaggtgcaccgcggcccggccaggcggagcgtcctcgccctgacccacgagggcaggttccacccg
ggcctggggtacgagcagcgctaccacagtggggacgtgcgcctcgacaccgtgggcagcgacgcct
accgcctctcagtgtcccgggctctgtctgccgaccagggctcctacaggtgtatcgtcagcgagtggatcg
ccgagcagggcaactggcaggaaatccaagaaaaggccgtggaagttgccaccgtggtgatccagcc
atcagttctgcgagcagctgtgcccaagaatgtgtctgtggctgaaggaaaggaactggacctgacctgta
acatcacaacagaccgagccgatgacgtccggcccgaggtgacgtggtccttcagcaggatgcctgac
agcaccctacctggctcccgcgtgttggcgcggcttgaccgtgattccctggtgcacagctcgcctcatgttg
ctttgagtcatgtggatgcacgctcctaccatttactggttcgggatgttagcaaagaaaactctggctactatt
actgccacgtgtccctgtgggcacccggacacaacaggagctggcacaaagtggcagaggccgtgtctt
ccccagctggtgtgggtgtgacctggctagaaccagactaccaggtgtacctgaatgcttccaaggtcccc
gggtttgcggatgaccccacagagctggcatgccgggtggtggacacgaagagtggggaggcgaatgt
ccgattcacggtttcgtggtactacaggatgaaccggcgcagcgacaatgtggtgaccagcgagctgctt
gcagtcatggacggggactggacgctaaaatatggagagaggagcaagcagcgggcccaggatgga
gactttattttttctaaggaacatacagacacgttcaatttccggatccaaaggactacagaggaagacaga
ggcaattattactgtgttgtgtctgcctggaccaaacagcggaacaacagctgggtgaaaagcaaggatg
tcttctccaagcctgttaacatattttgggcattagaagattccgtgcttgtggtgaaggcgaggcagccaaa
gcctttctttgctgccggaaatacatttgagatgacttgcaaagtatcttccaagaatattaagtcgccacgct
actctgttctcatcatggctgagaagcctgtcggcgacctctccagtcccaatgaaacgaagtacatcatct
ctctggaccaggattctgtggtgaagctggagaattggacagatgcatcacgggtggatggcgttgttttag
aaaaagtgcaggaggatgagttccgctatcgaatgtaccagactcaggtctcagacgcagggctgtacc
gctgcatggtgacagcctggtctcctgtcaggggcagcctttggcgagaagcagcaaccagtctctccaat
cctattgagatagacttccaaacctcaggtcctatatttaatgcttctgtgcattcagacacaccatcagtaatt
cggggagatctgatcaaattgttctgtatcatcactgtcgagggagcagcactggatccagatgacatggc
ctttgatgtgtcctggtttgcggtgcactcttttggcctggacaaggctcctgtgctcctgtcttccctggatcgga
agggcatcgtgaccacctcccggagggactggaagagcgacctcagcctggagcgcgtgagtgtgctg
gaattcttgctgcaagtgcatggctccgaggaccaggactttggcaactactactgttccgtgactccatggg
tgaagtcaccaacaggttcctggcagaaggaggcagagatccactccaagcccgtttttataactgtgaa
gatggatgtgctgaacgccttcaagtatcccttgctgatcggcgtcggtctgtccacggtcatcgggctcctgt
cctgtctcatcgggtactgcagctcccactggtgttgtaagaaggaggttcaggagacacggcgcgagcg
ccgcaggctcatgtcgatggagatggactag
SEQ ID No 8: Delta intein-CM
GCTCTTGCTGAGGGCACCAGAATCTTCGATCCCGTGACCGGCACCACA
CACAGAATCGAGGATGTCGTCGGCGGCAGAAAGCCCATTCATGTGGTG
GCTGCCGCCAAGGATGGAACCCTGCATGCCAGACCTGTGGTGTCTTGG
TTTGACCAGGGCACAAGAGATGTGATCGGCCTGAGAATTGCCGGCGGA
GCCATTCTGTGGGCCACACCAGATCACAAGGTGCTGACAGAGTACGGC
TGGCGAGCTGCTGGGGAACTGAGAAAGGGCGATAGAGTGGCCCAGCC
TAGAAGATTCGACGGCTTCGGAGACAGCGCCCCTATTCCTGCTAGAGT
GCAGGCTCTGGCCGACGCTCTGGACGACAAGTTTCTGCACGATATGCT
GGCCGAGGAACTGCGGTACAGCGTGATCAGAGAGGTGCTGCCTACCA
GACGGGCCAGAACCTTTGGCCTGGAAGTGGAAGAACTGCACACACTGG
TGGCCGAAGGCGTGGTGGTTCACAA
SEQ ID No 9: Foldon
GGGTCAGGTTATATACCAGAAGCCCCGAGAGACGGCCAGGCTTACGTT
AGAAAGGATGGCGAATGGGTACTTCTGTCCACCTTTCTTAGCCCTGCAA
ATAGG
SEQ ID No 10: Cre
ATGAGCAACCTGCTGACCGTGCACCAAAACCTGCCTGCTCTGCCTGTG
GATGCCACCTCTGATGAAGTGCGGAAGAACCTGATGGACATGTTCCGG
GACAGACAGGCCTTCAGCGAGCACACCTGGAAAATGCTGCTGAGCGTG
TGCAGATCTTGGGCCGCCTGGTGCAAGCTGAACAACAGAAAGTGGTTC
CCCGCCGAGCCTGAGGACGTGCGAGATTACCTGCTGTATCTGCAAGCC
AGAGGCCTGGCCGTGAAAACCATTCAGCAGCACCTGGGGCAGCTGAAT
ATGCTGCACAGAAGAAGCGGACTGCCCAGACCTAGCGATAGCAACGCC
GTGTCTCTGGTCATGCGGOGGATCAGAAAAGAAAACGTGGACGCCGGC
GAGAGAGCCAAACAGGCCCTGGCCTTTGAGAGAACCGACTTCGATCAA
GTGCGGTCCCTGATGGAAAACAGCGACAGGTGCCAGGATATCCGCAAT
CTGGCTTTCCTGGGAATCGCCTACAACACCCTGCTGCGGATTGCCGAG
ATCGCCCGGATCAGAGTGAAGGACATCAGCAGAACAGACGGCGGCAG
GATGCTGATCCACATCGGCAGAACTAAGACCCTGGTGTCCACCGCAGG
CGTGGAAAAAGCTCTGTCTCTGGGCGTGACCAAGCTGGTGGAAAGATG
GATCTCCGTGTCCGGCGTGGCCGACGATCCCAACAATTACCTGTTCTG
CAGAGTGCGCAAGAACGGCGTGGCAGCCCCTTCTGCTACAAGCCAGCT
GAGCACAAGAGCCCTGGAAGGCATCTTCGAGGCCACACACAGACTGAT
CTACGGCGCCAAGGACGACAGCGGCCAGAGATATCTTGCTTGGAGCGG
ACACTCCGCCAGAGTGGGAGCTGCTAGAGATATGGCTAGAGCCGGCGT
GTCAATCCCCGAGATTATGCAAGCTGGCGGCTGGACCAACGTGAACAT
CGTGATGAATTACATTCGGAACCTCGACAGCGAAACCGGCGCCATGGT
TCGACTGCTGGAAGATGGCGAT
SEQ ID No 11: Super suppressor of NFkB (SS)
ATGTTCCAAGCCGCCGAGAGGCCTCAAGAATGGGCCATGGAAGGCCCT
AGAGATGGCCTGAAGAAAGAGCGGCTGCTGGACGACAGACACGATGCC
GGACTGGACGCCATGAAGGACGAGGAATACGAGCAGATGGTCAAAGAG
CTGCAAGAGATCCGGCTGGAACCCCAAGAAGTGCCTAGAGGCTCTGAG
CCCTGGAAGCAGCAGCTGACAGAGGACGGCGATAGCTTTCTGCACCTG
GCCATCATCCACGAAGAGAAGGCCCTGACCATGGAAGTGATCCGGCAA
GTGAAAGGCGACCTGGCCTTCCTGAACTTCCAGAACAACCTGCAGCAG
ACCCCTCTGCATCTGGCCGTGATCACCAACCAGCCTGAGATTGCCGAA
GCTCTGCTCGGAGCCGGATGTGACCCTGAGCTGAGAGACTTCAGAGGC
AACACCCCTCTCCACCTGGCTTGCGAACAAGGCTGTCTGGCTTCTGTG
GGCGTGCTGACCCAGAGCTGTACCACACCTCATCTGCACAGCATCCTG
AAGGCCACCAACTACAACGGCCACACATGTCTGCACCTCGCCTCCATTC
ACGGCTACCTGGGAATCGTGGAACTGCTGGTGTCTCTGGGCGCCGACG
TGAACGCTCAAGAGCCTTGCAATGGCAGAACAGCCCTGCACCTTGCCG
TGGACCTGCAGAATCCTGATCTGGTGTCCCTGCTGCTGAAGTGCGGCG
CTGATGTGAACAGAGTGACCTACCAGGGCTACAGCCCCTACCAGCTTA
CATGGGGCAGACCCAGCACCAGAATCCAGCAGCAACTGGGCCAGCTGA
CCCTGGAAAACCTGCAGATGCTGCCCGAGAGCGAGGACGAAGAGAGCT
ACGATACCGAGTCCGAGTTCACAGAGTTCACCGAGGACGAGCTGCCCT
ACGACGATTGTGTGTTTGGCGGCCAGAGACTGACCCTG
SEQ ID No 12: Cas9
ATGGACGTCCACCACCACCATCATCACATGGCCCCCAAGAAGAAGAGA
AAGGTGGGCATCCACGGCGTGCCTGCCGCCGACAAGAAGTACTCCATC
GGCCTGGACATTGGCACCAACAGCGTGGGCTGGGCCGTGATTACCGAC
GAGTATAAGGTGCCCAGCAAGAAGTTCAAAGTGCTGGGAAACACCGAC
AGACACAGCATTAAGAAGAATCTGATCGGCGCCCTCCTGTTCGACAGC
GGCGAAACCGCCGAGGCCACCAGACTGAAGCGGACCGCCAGAAGACG
GTACACCAGAAGAAAGAACAGAATCTGCTACCTGCAGGAGATCTTCAGC
AACGAGATGGCCAAGGTGGATGACTCCTTCTTCCACAGACTGGAGGAG
TCCTTCCTGGTGGAGGAGGACAAAAAGCACGAGAGACACCCCATCTTC
GGAAACATCGTGGATGAGGTGGCCTACCACGAGAAATACCCCACCATC
TACCACCTGAGAAAGAAGCTGGTGGACAGCACCGACAAGGCTGACCTG
AGACTGATCTACCTGGCCCTGGCCCATATGATCAAGTTCAGAGGCCACT
TCCTGATTGAGGGCGACCTGAACCCCGACAACAGCGACGTGGACAAGC
TGTTCATCCAGCTGGTGCAGACCTACAACCAGCTGTTTGAGGAGAACCC
CATCAACGCTAGCGGCGTGGACGCCAAGGCCATTCTGTCCGCCAGGCT
GAGCAAAAGCAGGAGGCTGGAGAATCTCATCGCCCAGCTGCCTGGCGA
AAAGAAGAACGGCCTGTTCGGCAACCTGATCGCTCTGAGCCTGGGCCT
GACCCCCAACTTCAAAAGCAACTTCGACCTGGCCGAAGACGCCAAACT
GCAGCTGTCCAAGGACACCTACGACGACGACCTGGACAACCTGCTGGC
CCAGATCGGCGATCAGTACGCCGACCTGTTCCTGGCCGCCAAGAACTT
GAGCGACGCTATCCTGCTGAGCGACATCCTCAGAGTGAACACCGAGAT
CACCAAGGCCCCCCTGAGCGCCAGCATGATCAAAAGATACGACGAGCA
CCATCAGGACCTGACCCTGCTGAAGGCCCTGGTGAAACAGCAGCTGCC
CGAGAAGTACAAGGAAATCTTCTTCGACCAGAGCAAGAATGGCTACGCC
GGCTACATCGACGGCGGCGCCAGCCAGGAGGAGTTCTACAAGTTCATC
AAGCCCATCCTGGAAAAGATGGACGGCACCGAGGAGCTGCTGGTGAAG
CTGAACAGAGAGGATCTGCTCAGAAAGCAGAGAACCTTCGACAACGGC
AGCATCCCCCACCAGATCCACCTGGGCGAGCTGCACGCCATCCTGAGA
AGGCAGGAAGACTTCTACCCCTTCCTGAAGGACAATAGAGAGAAGATTG
AGAAGATCCTGACCTTCAGAATCCCCTACTACGTGGGCCCCCTGGCCA
GAGGCAACTCCAGATTCGCCTGGATGACCAGAAAGAGCGAGGAAACCA
TCACCCCCTGGAACTTCGAGGAGGTGGTCGACAAGGGCGCCTCCGCC
CAGAGCTTCATCGAGAGAATGACCAACTTTGACAAGAACCTGCCCAACG
AGAAGGTGCTGCCCAAGCACAGCCTGCTGTACGAGTATTTCACCGTGTA
CAACGAGCTGACCAAGGTGAAGTACGTGACCGAGGGCATGAGAAAGCC
CGCCTTCCTGTCCGGCGAGCAGAAGAAGGCAATCGTGGACCTGCTGTT
CAAGACCAATAGGAAGGTGACAGTGAAGCAGCTGAAGGAGGACTACTT
CAAGAAGATCGAGTGCTTCGACTCCGTGGAGATCTCTGGCGTGGAAGA
CAGATTCAACGCCAGCCTGGGAACCTACCATGATCTGCTGAAAATCATC
AAGGACAAGGACTTCCTGGATAACGAAGAGAACGAAGATATCCTGGAG
GACATCGTGCTGACTCTGACACTGTTCGAGGACAGAGAGATGATCGAG
GAGAGGCTGAAGACCTACGCCCACCTGTTTGACGACAAGGTGATGAAG
CAACTGAAGAGAAGGAGATACACCGGCTGGGGCAGACTGAGCAGGAA
GCTGATCAACGGCATCAGAGACAAGCAGAGCGGCAAGACCATCCTCGA
CTTCCTCAAGAGCGACGGCTTCGCCAACAGAAACTTCATGCAGCTGATC
CACGACGACAGCCTGACATTCAAGGAAGACATCCAGAAGGCTCAGGTG
AGCGGACAGGGCGATAGCCTGCATGAGCATATTGCCAATCTGGCCGGA
AGCCCTGCCATCAAGAAGGGCATCCTGCAGACCGTGAAGGTGGTGGAC
GAGCTCGTGAAGGTGATGGGCAGACACAAGCCCGAGAACATCGTGATC
GAGATGGCCAGGGAGAACCAGACCACCCAGAAGGGACAGAAGAACAG
CAGAGAGAGAATGAAGAGAATCGAGGAGGGCATCAAGGAGCTGGGCA
GCCAGATCTTGAAGGAGCACCCCGTGGAGAACACACAGCTCCAGAACG
AGAAACTGTACCTGTACTACCTGCAGAACGGCCGGGACATGTACGTGG
ACCAGGAGCTTGACATCAACAGACTGAGCGATTATGATGTGGACCACAT
CGTGCCCCAGAGCTTTCTGAAGGACGACAGCATCGACAACAAGGTGCT
GACCAGATCCGACAAGAACAGAGGCAAAAGCGACAACGTGCCCAGCGA
GGAGGTGGTGAAGAAGATGAAGAACTACTGGAGGCAGCTGCTGAACGC
TAAGCTCATCACCCAGAGAAAGTTCGACAACCTGACCAAGGCTGAGAG
GGGCGGCCTGAGCGAGCTGGACAAGGCCGGATTCATCAAGAGACAGC
TGGTGGAAACCAGACAGATCACCAAGCACGTGGCCCAGATCCTGGACT
CCAGAATGAACACCAAGTACGACGAGAACGACAAGCTGATCAGAGAAG
TGAAAGTGATCACACTGAAGTCCAAGCTGGTGAGCGACTTCAGGAAGG
ACTTCCAGTTCTACAAGGTGAGAGAGATCAACAACTACCACCACGCCCA
CGACGCCTACCTGAACGCCGTGGTGGGCACCGCCCTGATCAAAAAGTA
CCCTAAGCTGGAAAGCGAGTTCGTGTACGGCGACTACAAAGTGTACGA
CGTGAGGAAGATGATCGCCAAGAGCGAGCAGGAGATCGGCAAGGCCA
CCGCCAAGTATTTCTTCTACAGCAACATCATGAACTTCTTCAAGACCGAG
ATCACTCTGGCCAACGGGGAGATCAGGAAGAGGCCCCTGATCGAAACC
AACGGCGAAACCGGCGAGATCGTGTGGGACAAGGGCAGAGACTTCGC
CACCGTGAGAAAGGTGCTGTCCATGCCCCAGGTGAATATCGTGAAGAA
AACCGAGGTGCAGACCGGCGGATTCAGCAAGGAGAGCATCCTGCCCAA
GAGAAACAGGGACAAACTGATCGCTAGAAAGAAGGACTGGGACCCCAA
GAAGTACGGCGGTTTCGACAGCCCCACCGTGGCCTatAGCGTGCTGGT
GGTGGCCAAGGTGGAGAAAGGCAAGAGCAAAAAGCTGAAGAGCGTGAA
AGAGCTGCTGGGCATCACCATCATGGAGAGAAGCTCCTTCGAGAAGAA
CCCCATCGACTTCCTGGAGGCCAAGGGCTACAAGGAGGTGAAAAAGGA
CCTGATCATCAAGCTGCCTAAGTACAGCCTGTTTGAGCTGGAGAACGGC
AGAAAGAGAATGCTGGCCTCCGCCGGCGAGCTGCAGAAGGGCAACGA
GCTGGCCCTGCCTAGCAAGTACGTGAACTTCCTGTATCTGGCCAGCCA
CTACGAGAAGCTGAAAGGCAGCCCCGAGGACAACGAGCAGAAGCAGCT
GTTCGTGGAGCAGCACAAGCACTACCTGGACGAGATCATCGAGCAGAT
CTCCGAGTTCTCCAAGAGGGTGATCCTGGCCGACGCCAACCTGGACAA
AGTGCTGAGCGCCTACAACAAGCACAGAGACAAGCCCATCAGAGAGCA
GGCCGAGAATATCATCCACCTGTTCACCCTGACAAACCTGGGCGCCCC
TGCCGCCTTCAAGTACTTCGACACCACCATCGACCGGAAGAGATACACC
TCCACCAAAGAGGTGCTGGACGCCACCCTGATCCACCAGAGCATCACC
GGCCTGTACGAGACAAGAATCGATCTGAGCCAGCTGGGCGGCGATAAA
AGACCAGCCGCCACCAAGAAGGCCGGCCAGGCCAAGAAGAAGAAA

### Amino Acid Sequences

SEQ ID No 13: VSV-G
MKCLLYLAFLFIGVNCKFTIVFPHNQKGNWKNVPSNYHYCPSSSDLNWHN
DLIGTALQVKMPKSHKAIQADGWMCHASKWVTTCDFRWYGPKYITHSIRS
FTPSVEQCKESIEQTKQGTWLNPGFPPQSCGYATVTDAEAVIVQVTPHHV
LVDEYTGEWVDSQFINGKCSNYICPTVHNSTTWHSDYKVKGLCDSNLISM
DITFFSEDGELSSLGKEGTGFRSNYFAYETGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGTTTERELWDDWAPYEDVEIGPNGVLRTSSGYKFPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK
SEQ ID No 14: CVG
MNFLLLTFIVLPLCSHAKFSIVFPQSQKGNWKNVPSSYHYCPSSSDQNWH
NDLLGITMKVKMPKTHKAIQADGWMCHAAKWITTCDFRWYGPKYITHSIH
SIQPTSEQCKESIKQTKQGTWMSPGFPPQNCGYATVTDSVAVVVQATPHH
VLVDEYTGEWIDSQFPNGKCETEECETVHNSTVWYSDYKVTGLCDATLVD
TEITFFSEDGKKESIGKPNTGYRSNYFAYEKGDKVCKMNYCKHAGVRLPS
GVWFEFVDQDVYAAAKLPECPVGATISAPTQTSVDVSLILDVERILDYSL
CQETWSKIRSKQPVSPVDLSYLAPKNPGTGPAFTIINGTLKYFETRYIRI
DIDNPIISKMVGKISGSQTERELWTEWFPYEGVEIGPNGILKTPTGYKFP
LFMIGHGMLDSDLHKTSQAEVFEHPHLAEAPKQLPEEETLFFGDTGISKN
PVELIEGWFSSWKSTVVTFFFAIGVFILLYVVARIVIAVRYRYQGSNNKR
IYNDIEMSRFRKVSGWRLFKKIS
SEQ ID No 15: PFV
MAPPMTLQQWIIWKKMNKAHEALQNTTTVTEQQKEQIILDIQNEEVQPTR
RDKFRYLLYTCCATSSRVLAWMFLVCILLIIVLVSCFVTISRIQWNKDIQ
VLGPVIDWNVTQRAVYQPLQTRRIARSLRMQHPVPKYVEVNMTSIPQGVY
YEPHPEPIVVKERVLGLSQILMINSENIANNANLTQEVKKLLTEMVNEEM
QSLSDVMIDFEIPLGDPRDQEQYIHRKCYQEFANCYLVKYKEPKPWPKEG
LIADQCPLPGYHAGLTYNRQSIWDYYIKVESIRPANWTTKSKYGQARLGS
FYIPSSLRQINVSHVLFCSDQLYSKWYNIENTIEQNERFLLNKLNNLTSG
TSVLKKRALPKDWSSQGKNALFREINVLDICSKPESVILLNTSYYSFSLW
EGDCNFTKDMISQLVPECDGFYNNSKWMHMHPYACRFWRSKNEKEETKCR
DGETKRCLYYPLWDSPESTYDFGYLAYQKNFPSPICIEQQKIRDQDYEVY
SLYQECKIASKAYGIDTVLFSLKNFLNYTGTPVNEMPNARAFVGLIDPKF
PPSYPNVTREHYTSCNNRKRRSVDNNYAKLRSMGYALTGAVQTLSQISDI
NDENLQQGIYLLRDHVITLMEATLHDISVMEGMFAVQHLHTHLNHLKTML
LERRIDWTYMSSTWLQQQLQKSDDEMKVIKRIARSLVYYVKQTHSSPTAT
AWEIGLYYELVIPKHIYLNNWNVVNIGHLVKSAGQLTHVTIAHPYEIINK
ECVETIYLHLEDCTRQDYVICDVVKIVQPCGNSSDTSDCPVWAEAVKEPF
VQVNPLKNGSYLVLASSTDCQIPPYVPSIVTVNETTSCFGLDFKRPLVAE
ERLSFEPRLPNLQLRLPHLVGIIAKIKGIKIEVTSSGESIKEQIERAKAE
LLRLDIHEGDTPAWIQQLAAATKDVWPAAASALQGIGNFLSGTAQGIFGT
AFSLLGYLKPILIGVGVILLVILIFKIVSWIPTKKKNQ
SEQ ID No 16: CD63
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVFRDKVMSEFNNNFRQQMENYPKNNHTASILDRMQADFKCCGAAN
YTDWEKIPSMSKNRVPDSCCINVTVGCGINFNEKAIHKEGCVEKIGGWLR
KNVLVVAAAALGIAFVEVLGIVFACCLVKSIRSGYEVM
SEQ ID No 17: CD9
MPVKGGTKCIKYLLFGFNFIFWLAGIAVLAIGLWLRFDSQTKSIFEQETN
NNNSSFYTGVYILIGAGALMMLVGFLGCCGAVQESQCMLGLFFGFLLVIF
AIEIAAAIWGYSHKDEVIKEVQEFYKDTYNKLKTKDEPQRETLKAIHYAL
NCCGLAGGVEQFISDICPKKDVLETFTVKSCPDAIKEVFDNKFHIIGAVG
IGIAVVMIFGMIFSMILCCAIRRNREMV
SEQ ID No 18: CD81
MGVEGCTKCIKYLLFVFNFVFWLAGGVILGVALWLRHDPQTTNLLYLELG
DKPAPNTFYVGIYILIAVGAVMMFVGFLGCYGAIQESQCLLGTFFTCLVI
LFACEVAAGIWGFVNKDQIAKDVKQFYDQALQQAVVDDDANNAKAVVKTF
HETLDCCGSSTLTALTTSVLKNNLCPSGSNIISNLFKEDCHQKIDDLFSG
KLYLIGIAAIVVAVIMIFEMILSMVLCCGIRNSSVY
SEQ ID No 19: PTGFRN
MGRLASRPLLLALLSLALCRGRVVRVPTATLVRVVGTELVIPCNVSDYDG
PSEQNFDWSFSSLGSSFVELASTWEVGFPAQLYQERLQRGEILLRRTAND
AVELHIKNVQPSDQGHYKCSTPSTDATVQGNYEDTVQVKVLADSLHVGPS
ARPPPSLSLREGEPFELRCTAASASPLHTHLALLWEVHRGPARRSVLALT
HEGRFHPGLGYEQRYHSGDVRLDTVGSDAYRLSVSRALSADQGSYRCIVS
EWIAEQGNWQEIQEKAVEVATVVIQPSVLRAAVPKNVSVAEGKELDLTCN
ITTDRADDVRPEVTWSFSRMPDSTLPGSRVLARLDRDSLVHSSPHVALSH
VDARSYHLLVRDVSKENSGYYYCHVSLWAPGHNRSWHKVAEAVSSPAGVG
VTWLEPDYQVYLNASKVPGFADDPTELACRVVDTKSGEANVRFTVSWYYR
MNRRSDNVVTSELLAVMDGDWTLKYGERSKQRAQDGDFIFSKEHTDTFNF
RIQRTTEEDRGNYYCVVSAWTKQRNNSWVKSKDVFSKPVNIFWALEDSVL
VVKARQPKPFFAAGNTFEMTCKVSSKNIKSPRYSVLIMAEKPVGDLSSPN
ETKYIISLDQDSVVKLENWTDASRVDGVVLEKVQEDEFRYRMYQTQVSDA
GLYRCMVTAWSPVRGSLWREAATSLSNPIEIDFQTSGPIFNASVHSDTPS
VIRGDLIKLFCIITVEGAALDPDDMAFDVSWFAVHSFGLDKAPVLLSSLD
RKGIVTTSRRDWKSDLSLERVSVLEFLLQVHGSEDQDFGNYYCSVTPWVK
SPTGSWQKEAEIHSKPVFITVKMDVLNAFKYPLLIGVGLSTVIGLLSCLI
GYCSSHWCCKKEVQETRRERRRLMSMEMD*
SEQ ID No 20: Delta intein-CM
ALAEGTRIFDPVTGTTHRIEDVVGGRKPIHVVAAAKDGTLHARPVVSWFD
QGTRDVIGLRIAGGAILWATPDHKVLTEYGWRAAGELRKGDRVAQPRRFD
GFGDSAPIPARVQALADALDDKFLHDMLAEELRYSVIREVLPTRRARTFG
LEVEELHTLVAEGVVVHN
SEQ ID No 21: Foldon
GSGYIPEAPRDGQAYVRKDGEWVLLSTFLSPANR
SEQ ID No 22: Cre
MSNLLTVHQNLPALPVDATSDEVRKNLMDMFRDRQAFSEHTWKMLLSVCR
SWAAWCKLNNRKWFPAEPEDVRDYLLYLQARGLAVKTIQQHLGQLNMLHR
RSGLPRPSDSNAVSLVMRRIRKENVDAGERAKQALAFERTDFDQVRSLME
NSDRCQDIRNLAFLGIAYNTLLRIAEIARIRVKDISRTDGGRMLIHIGRT
KTLVSTAGVEKALSLGVTKLVERWISVSGVADDPNNYLFCRVRKNGVAAP
SATSQLSTRALEGIFEATHRLIYGAKDDSGQRYLAWSGHSARVGAARDMA
RAGVSIPEIMQAGGWTNVNIVMNYIRNLDSETGAMVRLLEDGD
SEQ ID No 23: Super suppressor of NFkB (SS)
MFQAAERPQEWAMEGPRDGLKKERLLDDRHDAGLDAMKDEEYEQMVKELQ
EIRLEPQEVPRGSEPWKQQLTEDGDSFLHLAIIHEEKALTMEVIRQVKGD
LAFLNFQNNLQQTPLHLAVITNQPEIAEALLGAGCDPELRDFRGNTPLHL
ACEQGCLASVGVLTQSCTTPHLHSILKATNYNGHTCLHLASIHGYLGIVE
LLVSLGADVNAQEPCNGRTALHLAVDLQNPDLVSLLLKCGADVNRVTYQG
YSPYQLTWGRPSTRIQQQLGQLTLENLQMLPESEDEESYDTESEFTEFTE
DELPYDDCVFGGQRLTL
SEQ ID No 24: Cas9
MDVHHHHHHMAPKKKRKVGIHGVPAADKKYSIGLDIGTNSVGWAVITDEY
KVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRY
DEHHQDLTLLKALVKQQLPEKYKEIFFDQSKNGYAGYIDGGASQEEFYKF
IKPILEKMDGTEELLVKLNREDLLRKQRTFDNGSIPHQIHLGELHAILRR
QEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTL
FEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWD
KGRDFATVRKVLSMPQVNIVKKTEVQTGGFSKESILPKRNRDKLIARKKD
WDPKKYGGFDSPTVAYSVLVVAKVEKGKSKKLKSVKELLGITIMERSSFE
KNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFK
YFDTTIDRKRYTSTKEVLDATLIHQSITGLYETRIDLSQLGGDKRPAATK
KAGQAKKKK

1. An engineered extracellular vesicle (EV) for delivery of a bioactive cargo, the engineered EV comprising:
(i) an endosomal escape enhancer; and,
(ii) a cargo.

2. An engineered EV according to claim 1 wherein, the engineered EV further comprises a release system.

3. An engineered EV according to claim 1 or claim 2, wherein the engineered EV further comprises an EV protein, preferably wherein the EV protein forms a fusion protein with the cargo and, where present, the release system.

4. An engineered EV according to claim 1 or claim 2, wherein the engineered EV further comprises a multimerization domain, preferably a trimerization domain, and most preferably foldon.

5. An engineered EV according to any one of the preceding claims wherein the endosomal escape enhancer is selected from Vesicular Stomatitis Virus Glycoprotein (VSVG), cocal virus Glycoprotein (CVG), Prototype Foamy Virus (PFV) Envelope and human-derived virus like proteins (heVLPs).

6. An engineered EV according to claim 3, wherein the EV protein is selected from an EV transmembrane protein and an EV membrane associated protein.

7. An engineered EV according to claim 6, wherein the EV transmembrane protein is a single-pass transmembrane protein or a multi-pass transmembrane protein.

8. An engineered EV according to claim 6 or claim 7, wherein the EV transmembrane protein is a tetraspanin, preferably a tetraspanin selected from CD63, CD9, CD81 and derivatives, domains, variants, mutants, or regions thereof.

9. An engineered EV according to any one of the preceding claims, wherein, when the EV comprises the release system, the release system is a self-cleaving protein.

10. An engineered EV according to claim 9, wherein the self-cleaving protein is an intein or a derivative, domain, variant, mutant or region thereof.

11. An engineered EV according to claim 10, wherein the intein is a mini-intein, preferably a mini-intein that has been modified to optimise the cleavage rate, and more preferably delta-intein-CM.

12. An engineered EV according to any one of the preceding claims, wherein the cargo is one or more of a protein, an enzyme, a CRISPR protein or a nucleic acid binding protein, preferably wherein the CRISPR protein is Cas9, preferably wherein the enzyme is a meganuclease.

13. At least two plasmids, each plasmid comprising a polynucleotide construct, wherein:
(i) the polynucleotide construct of the first plasmid encodes a protein construct, the protein construct comprising an endosomal escape enhancer, preferably VSV-G, and
(ii) the polynucleotide construct of the second plasmid encodes a protein construct, the protein construct comprising a cargo, an EV protein and optionally a release system, wherein the release system is preferably a self-cleaving protein, more preferably intein.

14. A plasmid comprising a polynucleotide construct that encodes a protein construct, the protein construct comprising an endosomal escape enhancer, a multimerization domain, and optionally a release system, preferably wherein the endosomal escape enhancer is VSV-G, the multimerization domain is foldon and/or the release system is a self-cleaving intein, preferably intein.

15. A cell comprising at least two plasmids according to claim 13 or a plasmid according to claim 14.

16. A protein construct for use in a nanoparticle, wherein the protein construct comprises an endosomal escape enhancer, a multimerization domain, and optionally a release system, preferably wherein the nanoparticle is an EV, preferably an exosome, the endosomal escape enhancer is VSV-G, the multimerization domain is foldon and/or the release system is a self-cleaving protein, preferably intein.

17. A method of making an engineered EV according to any one of claims 1 to 12, wherein the method comprises the steps of:
(i) introducing into an EV-producing cell a polynucleotide construct encoding an endosomal escape enhancer;
(ii) expressing the polynucleotide construct in the EV-producing cell; and
(iii) loading a cargo into the EV;
thereby generating an EV comprising both an endosomal escape enhancer and a cargo.

18. A method according to claim 17, wherein the endosomal escape enhancer is displayed on the surface of the EV and the cargo is loaded into the lumen of the EV.

19. An in vitro method for assaying engineered EVs according to any one of claims 1 to 12, wherein the method comprises the steps of:
(i) co-culturing reporter cells and EV-producing cells capable of producing the engineered EVs; and
(ii) measuring signal-positive cells.

20. The in vitro method according to claim 19, wherein the reporter cells comprise an above average receptor level expression, preferably VSV-G (LDL-R) receptor level expression.

21. The in vitro method according to claim 20, wherein the above average receptor level expression is from at least about 51%, and preferably at least about 55%, of the overall expression profile.

22. The in vitro method according to any one of claims 19 to 21, wherein the EV-producing cells are HEK293T cells.

23. The in vitro method according to any one of claims 19 to 22, wherein the method comprises a delivery saturation between a cell-to-cell ratio of from about 1:5 to about 1:1 EV-producing cells to reporter cells.

24. The in vitro method according to any one of claims 19 to 23, wherein the method comprises a detection limitation between a cell-to-cell ratio of from at least about 30:1 EV-producing cells to reporter cells, preferably from about 30:1 to about 50:1, more preferably from about 30:1 to about 50:1, most preferably from at least about 50:1 when the reporter cell is B16F10.

25. A composition comprising an engineered EV according to any one of claims 1 to 12 and an excipient, diluent, vehicle, solvent and/or carrier.

26. An engineered EV according to any one of claims 1 to 12 or a composition according to claim 25 for use as a medicament.

27. An engineered EV or composition according to claim 26 for use in the treatment of cancer and/or brain-related conditions disorders and/or diseases.


SEQUENCE LISTING (24 sequences)

SEQ ID NO: 1 - DNA, 1533 bp, synthetic construct
  source 1..1533: mol_type = other DNA
atgaagtgtctcctttatctcgcctttctctttataggggtcaactgcaaatttacgatcgtttttccgcacaatcagaaaggaaactggaaaaatgtgccatctaactaccattactgcccttctagctctgaccttaactggcataatgatttgatcgggacagcgctccaagtcaaaatgccgaaatcccacaaggcgatccaagcggatggctggatgtgtcatgcgtccaaatgggttacaacttgcgatttccggtggtacgggcccaaatacatcacccacagtattagaagctttacacctagtgtagaacagtgcaaagagagcatcgaacaaaccaaacagggcacttggctcaatccaggctttcccccgcagtcttgcggctacgccactgtgaccgatgcggaggccgtgatcgtccaagtcaccccccatcacgtcctggttgacgaatatacaggggagtgggtagatagtcaatttatcaacggtaaatgcagtaattatatatgccccaccgtgcataattctaccacatggcacagcgattacaaggtgaaagggttgtgcgattcaaacctcatatcaatggacatcaccttcttttcagaagatggtgagctcagttctcttgggaaggaaggtactgggtttcggagtaattacttcgcctacgaaacgggtggcaaggcctgcaagatgcagtactgcaaacactggggggtccgcctgccaagtggggtttggttcgaaatggcggacaaggatttgtttgcggcggcgcggttcccagagtgtccagaagggtcatccatatctgcgccttctcaaacgtctgtggacgtgtctcttatccaagacgtggagaggattttggattactcactttgccaagaaacgtggagtaagataagagcgggtctgccaataagtccggtggatcttagttacctggcaccaaaaaacccaggcaccgggcccgccttcactatcataaacgggactctgaaatattttgagacgagatatatccgggttgatatcgctgcgccgatactctccaggatggtcggcatgattagtggtacgacgactgagcgagaactgtgggatgactgggctccgtacgaagacgttgagatcggtccaaacggggtcttgcgcacatctagtggttataagttcccgctgtatatgattggtcacggtatgcttgactcagacctccatctcagttccaaggctcaagtgtttgagcacccacacatccaagatgcggcaagtcaacttcctgacgatgagtcactgttctttggggacactggactctccaagaatccaattgagttggttgaaggatggttttcatcatggaaaagtagtatagcctcttttttcttcataattggacttatcataggtttgttcttggtactcagagtcggtatacacctgtgtataaaattgaagcacactaagaaaaggcaaatctatacggacatagaaatgaatcgattggggaag

SEQ ID NO: 2 - DNA, 1569 bp, synthetic construct
  source 1..1569: mol_type = other DNA
atgaattttctccttctgacatttatcgtactgccactgtgttctcatgctaagttcagtatcgtgttcccccagagtcagaagggtaattggaaaaatgtgccctcctcataccactactgccccagttcatcagaccagaactggcataacgacctgcttgggatcacaatgaaggttaaaatgcccaaaacacacaaggctatccaggccgatggctggatgtgccatgctgccaagtggatcaccacctgcgattttcgctggtacgggccaaaatacatcactcattcaattcactcaatacagcccacctctgaacagtgcaaggagtctattaagcaaaccaagcagggcacatggatgagccccggctttccacctcagaattgcgggtacgcaacagtgaccgactctgtggcagtcgttgtgcaagcgaccccccatcatgtcctggtggacgaatacaccggcgagtggatagattctcagttcccaaatggcaaatgcgagactgaagagtgtgagacagtgcataactcaacagtgtggtacagcgactataaggtaaccggtttgtgtgacgcaaccctggtggataccgaaatcacgttcttctctgaagacggaaagaaggaatccatcggcaagcccaacacaggttaccgttccaactacttcgcatatgaaaagggcgataaagtatgtaagatgaactactgtaagcatgcaggcgttcgacttcctagtggcgtgtggttcgaatttgtggatcaggatgtttatgctgccgccaagttacctgaatgccccgtgggcgccactattagtgccccaacacagacatccgtggatgtgtcattaatccttgacgtggaaagaatcctggactactcactctgtcaggagacctggtctaagatccggagcaagcagcctgtaagtcccgtagacctctcttatctggccccaaaaaacccgggcacagggcctgccttcaccataatcaacggcacactgaagtacttcgagactcgctatattaggattgacatcgacaacccaataatctcaaaaatggtggggaagattagcggatcacagacagagagggagctgtggacagagtggtttccttacgaaggcgtagaaattggcccgaacggaatcctcaagacccctactggatataagtttcctctgtttatgatcggacacggcatgcttgacagcgacctgcataagacctctcaggccgaggtcttcgagcacccccacctggcggaggctcccaagcagctgccagaggaagagacattgttcttcggcgacacagggataagcaaaaaccctgtcgaactgattgagggctggttcagctcatggaaatcaaccgtcgtgacattcttcttcgcaataggtgtgttcatactgctctatgtcgtcgctcggattgttatcgctgttagataccgctaccaaggctcaaacaataagagaatctacaacgacatcgagatgagcaggtttagaaaggtgagcggctggcggctgttcaagaagattagc

SEQ ID NO: 3 - DNA, 2964 bp, synthetic construct
  source 1..2964: mol_type = other DNA
atggcccctcccatgaccctgcagcagtggatcatctggaagaagatgaacaaggcccacgaggccctgcagaacaccaccaccgtgaccgagcagcagaaagagcagatcatcctggacatccagaacgaggaagtgcagcccaccaggcgggacaagttcagatacctgctgtacacctgctgcgccacctccagccgggtgctggcctggatgttcctggtgtgcatcctgctgatcatcgtgctggtgtcctgcttcgtgaccatcagccggatccagtggaacaaggacatccaggtgctgggccccgtgatcgactggaacgtgacccagcgggccgtgtaccagcccctgcagacccggcggatcgcccggtccctgcggatgcagcaccccgtgcccaagtacgtggaggtgaacatgaccagcatcccccagggcgtgtactacgagccccaccccgagcccatcgtggtgaaagaaagagtgctgggcctgagccagatcctgatgatcaacagcgagaacatcgccaacaacgccaacctgacccaggaagtgaagaaactgctgaccgagatggtgaacgaagagatgcagagcctgagcgacgtgatgatcgacttcgagatccccctgggcgaccccagggaccaggaacagtacatccaccggaagtgctaccaggaatttgccaactgctacctggtgaagtacaaagagcccaagccctggcccaaagagggcctgatcgccgaccagtgccccctgcccggctatcacgccggcctgacctacaaccggcagagcatctgggactactacatcaaggtggagagcatcaggcccgccaactggaccaccaagagcaagtacggccaggcccggctgggcagcttctacatccccagcagcctgcggcagatcaacgtgagccacgtgctgttctgcagcgaccagctgtacagcaagtggtacaacatcgagaacaccatcgagcagaacgagcggttcctgctgaacaagctgaataacctgaccagcggcaccagcgtgctgaagaagagagccctgcccaaggactggtccagccagggcaagaacgccctgttccgggagatcaatgtgctggacatctgcagcaagcccgagagcgtgatcctgctgaataccagctactacagcttcagcctgtgggagggcgactgcaacttcaccaaggacatgatcagccagctggtgcccgagtgcgacggcttctacaacaactccaagtggatgcacatgcacccctacgcctgccggttctggcggagcaagaacgagaaagaggaaaccaagtgccgggacggcgagaccaagcggtgcctgtactaccccctgtgggacagccctgagagcacctacgacttcggctacctggcctaccagaagaacttccccagccccatctgcatcgaacagcagaagatccgggaccaggactacgaggtgtacagcctgtaccaggaatgcaagatcgccagcaaggcctacggcatcgacaccgtgctgttcagcctgaagaatttcctgaactacaccggcacccccgtgaacgagatgcccaacgccagggccttcgtgggcctgattgaccccaagttcccccccagctaccccaacgtgacccgggagcactacaccagctgcaacaaccggaagcggcggagcgtggacaacaactacgccaagctgcggagcatgggctacgctctgacaggcgccgtgcagaccctgtcccagatcagcgacatcaacgacgagaacctgcagcagggcatctacctgctgcgggaccacgtgatcaccctgatggaagccaccctgcacgacatcagcgtgatggaaggcatgttcgccgtgcagcacctgcacacccacctgaatcacctgaaaaccatgctgctggaacggcgcatcgactggacctacatgagcagcacctggctgcagcagcagctgcagaaaagcgacgacgagatgaaggtgatcaagcggatcgccagatctctggtgtactacgtgaagcagacccacagcagccccaccgccaccgcctgggagatcggcctgtactatgagctggtgatccccaagcacatctacctgaacaactggaatgtggtgaacatcggccacctggtgaaaagcgccggacagctgacccacgtgaccatcgcccacccctacgagatcatcaacaaagaatgcgtggagaccatctatctgcacctggaagattgcacccggcaggactacgtgatctgcgacgtggtgaagatcgtgcagccctgcggcaacagcagcgacaccagcgactgccccgtgtgggccgaggccgtgaaagaacccttcgtgcaggtgaaccccctgaagaacggctcctacctggtgctggccagcagcaccgactgccagatccccccctacgtgcccagcatcgtgaccgtgaatgagaccacctcctgcttcggcctggacttcaagcggcccctggtggccgaggaaagactgagcttcgagccccggctgcccaacctgcagctgaggctgccccacctggtgggcatcatcgccaagatcaagggcatcaagatcgaggtgaccagcagcggcgagagcatcaaagaacagatcgagcgggccaaggccgagctgctgcggctggatatccacgagggcgacacacccgcctggatccagcagctggccgccgccaccaaggacgtgtggcccgctgcagccagcgccctgcagggcatcggcaactttctgagcggcaccgcccagggcatcttcggcaccgccttctccctgctgggctacctgaagcccatcctgatcggcgtgggcgtgattctgctggtgattctgatcttcaagatcgtgagctggatccccaccaagaaaaagaaccag

SEQ ID NO: 4 - DNA, 714 bp, synthetic construct
  source 1..714: mol_type = other DNA
atggctgtggaaggcggaatgaagtgcgtgaagttcctgctgtacgtgctgctgctggccttctgtgcttgtgccgtgggactgattgccgttggagttggagcacagctggtgctgagccagacaatcatccagggcgccacacctggatctctgctgcccgtggttattatcgccgtgggcgtgttcctgttcctggtggcctttgtgggatgttgcggcgcctgcaaagaaaactactgcctgatgattaccttcgccatcttcctgagcctgatcatgctggtggaagtggccgctgccattgccggctacgtgttcagagacaaagtgatgagcgagttcaacaacaacttccggcagcagatggaaaactaccccaagaacaaccacaccgccagcatcctggacagaatgcaggccgacttcaagtgttgcggagccgccaactacaccgactgggagaagatccccagcatgagcaagaacagagtgcccgacagctgctgcatcaacgtgacagttggctgcggcatcaatttcaacgagaaggccatccacaaagagggctgcgtcgagaagatcggcggctggctgagaaagaacgtgctggttgttgccgccgctgctctgggaattgccttcgtggaagtgctgggcatcgtgttcgcctgctgtctggtcaagagcatccgcagcggctacgaagtgatg

SEQ ID NO: 5 - DNA, 684 bp, synthetic construct
  source 1..684: mol_type = other DNA
atgcctgtcaagggcggtacaaaatgcatcaagtaccttttgtttggttttaatttcatcttctggcttgcaggcatagctgtgctggccatcgggctttggctgcggtttgatagccaaacaaagagtatctttgagcaggaaactaataataacaatagtagtttttatactggcgtctatattcttattggtgcaggagccctcatgatgcttgtcggctttctggggtgttgtggggccgttcaggaatcccagtgtatgcttggacttttctttgggttcctgctcgttatcttcgccatcgagatcgcagcggcgatatggggttactcacataaggacgaagtaatcaaggaagttcaggagttctataaagacacgtataataaactgaaaaccaaagatgagccccaacgggagacccttaaagcgatccactacgctctcaactgctgcggacttgcaggtggcgttgaacaattcatatccgacatctgcccgaaaaaggatgtactcgaaacttttactgtcaagtcatgtccggacgctataaaggaagtgttcgataataagtttcacataatcggcgcagttgggataggaatagccgtagtgatgatatttggcatgatttttagtatgatattgtgttgtgccataaggaggaatagggagatggta

SEQ ID NO: 6 - DNA, 708 bp, synthetic construct
  source 1..708: mol_type = other DNA
atgggtgttgaagggtgtacgaagtgtattaaatatcttttgtttgtattcaacttcgtattttggcttgctggcggagtaattctgggggtagcactgtggctgcggcacgacccccaaacgactaacttgctttatctggaattgggcgacaaaccggcgcccaacacgttctacgtcggtatttatatccttatcgcagtaggggctgttatgatgttcgtaggttttctggggtgttacggcgcaatacaggagtcacagtgtctgctcggtacgttttttacttgtctcgtaattcttttcgcctgcgaagtcgcggcaggtatctggggttttgtaaacaaagatcaaatagcaaaggacgtaaaacaattttatgaccaagctctgcagcaggcggttgtagacgatgacgcgaacaacgctaaagccgttgtcaagactttccatgaaacactcgactgttgcggctccagtacacttaccgcactgaccacgtcagtgctgaaaaacaacctgtgccccagtggctcaaacatcatatctaacttgttcaaggaagattgccaccaaaagattgacgatctgttttcaggcaaattgtatctcattgggattgccgcgatcgttgtcgcagtgattatgatctttgagatgatcctgagcatggtactttgctgtggtataagaaattcttctgtttat

SEQ ID NO: 7 - DNA, 2640 bp, synthetic construct
  source 1..2640: mol_type = other DNA
atggggcgcctggcctcgaggccgctgctgctggcgctcctgtcgttggctctttgccgagggcgtgtggtgagagtccccacagcgaccctggttcgagtggtgggcactgagctggtcatcccctgcaacgtcagtgactatgatggccccagcgagcaaaactttgactggagcttctcatctttggggagcagctttgtggagcttgcaagcacctgggaggtggggttcccagcccagctgtaccaggagcggctgcagaggggcgagatcctgttaaggcggactgccaacgacgccgtggagctccacataaagaacgtccagccttcagaccaaggccactacaaatgttcaacccccagcacagatgccactgtccagggaaactatgaggacacagtgcaggttaaagtgctggccgactccctgcacgtgggccccagcgcgcggcccccgccgagcctgagcctgcgggagggggagcccttcgagctgcgctgcaccgccgcctccgcctcgccgctgcacacgcacctggcgctgctgtgggaggtgcaccgcggcccggccaggcggagcgtcctcgccctgacccacgagggcaggttccacccgggcctggggtacgagcagcgctaccacagtggggacgtgcgcctcgacaccgtgggcagcgacgcctaccgcctctcagtgtcccgggctctgtctgccgaccagggctcctacaggtgtatcgtcagcgagtggatcgccgagcagggcaactggcaggaaatccaagaaaaggccgtggaagttgccaccgtggtgatccagccatcagttctgcgagcagctgtgcccaagaatgtgtctgtggctgaaggaaaggaactggacctgacctgtaacatcacaacagaccgagccgatgacgtccggcccgaggtgacgtggtccttcagcaggatgcctgacagcaccctacctggctcccgcgtgttggcgcggcttgaccgtgattccctggtgcacagctcgcctcatgttgctttgagtcatgtggatgcacgctcctaccatttactggttcgggatgttagcaaagaaaactctggctactattactgccacgtgtccctgtgggcacccggacacaacaggagctggcacaaagtggcagaggccgtgtcttccccagctggtgtgggtgtgacctggctagaaccagactaccaggtgtacctgaatgcttccaaggtccccgggtttgcggatgaccccacagagctggcatgccgggtggtggacacgaagagtggggaggcgaatgtccgattcacggtttcgtggtactacaggatgaaccggcgcagcgacaatgtggtgaccagcgagctgcttgcagtcatggacggggactggacgctaaaatatggagagaggagcaagcagcgggcccaggatggagactttattttttctaaggaacatacagacacgttcaatttccggatccaaaggactacagaggaagacagaggcaattattactgtgttgtgtctgcctggaccaaacagcggaacaacagctgggtgaaaagcaaggatgtcttctccaagcctgttaacatattttgggcattagaagattccgtgcttgtggtgaaggcgaggcagccaaagcctttctttgctgccggaaatacatttgagatgacttgcaaagtatcttccaagaatattaagtcgccacgctactctgttctcatcatggctgagaagcctgtcggcgacctctccagtcccaatgaaacgaagtacatcatctctctggaccaggattctgtggtgaagctggagaattggacagatgcatcacgggtggatggcgttgttttagaaaaagtgcaggaggatgagttccgctatcgaatgtaccagactcaggtctcagacgcagggctgtaccgctgcatggtgacagcctggtctcctgtcaggggcagcctttggcgagaagcagcaaccagtctctccaatcctattgagatagacttccaaacctcaggtcctatatttaatgcttctgtgcattcagacacaccatcagtaattcggggagatctgatcaaattgttctgtatcatcactgtcgagggagcagcactggatccagatgacatggcctttgatgtgtcctggtttgcggtgcactcttttggcctggacaaggctcctgtgctcctgtcttccctggatcggaagggcatcgtgaccacctcccggagggactggaagagcgacctcagcctggagcgcgtgagtgtgctggaattcttgctgcaagtgcatggctccgaggaccaggactttggcaactactactgttccgtgactccatgggtgaagtcaccaacaggttcctggcagaaggaggcagagatccactccaagcccgtttttataactgtgaagatggatgtgctgaacgccttcaagtatcccttgctgatcggcgtcggtctgtccacggtcatcgggctcctgtcctgtctcatcgggtactgcagctcccactggtgttgtaagaaggaggttcaggagacacggcgcgagcgccgcaggctcatgtcgatggagatggactag

SEQ ID NO: 8 - DNA, 503 bp, synthetic construct
  source 1..503: mol_type = other DNA
gctcttgctgagggcaccagaatcttcgatcccgtgaccggcaccacacacagaatcgaggatgtcgtcggcggcagaaagcccattcatgtggtggctgccgccaaggatggaaccctgcatgccagacctgtggtgtcttggtttgaccagggcacaagagatgtgatcggcctgagaattgccggcggagccattctgtgggccacaccagatcacaaggtgctgacagagtacggctggcgagctgctggggaactgagaaagggcgatagagtggcccagcctagaagattcgacggcttcggagacagcgcccctattcctgctagagtgcaggctctggccgacgctctggacgacaagtttctgcacgatatgctggccgaggaactgcggtacagcgtgatcagagaggtgctgcctaccagacgggccagaacctttggcctggaagtggaagaactgcacacactggtggccgaaggcgtggtggttcacaa

SEQ ID NO: 9 - DNA, 102 bp, synthetic construct
  source 1..102: mol_type = other DNA
gggtcaggttatataccagaagccccgagagacggccaggcttacgttagaaaggatggcgaatgggtacttctgtccacctttcttagccctgcaaatagg

SEQ ID NO: 10 - DNA, 1029 bp, synthetic construct
  source 1..1029: mol_type = other DNA
atgagcaacctgctgaccgtgcaccaaaacctgcctgctctgcctgtggatgccacctctgatgaagtgcggaagaacctgatggacatgttccgggacagacaggccttcagcgagcacacctggaaaatgctgctgagcgtgtgcagatcttgggccgcctggtgcaagctgaacaacagaaagtggttccccgccgagcctgaggacgtgcgagattacctgctgtatctgcaagccagaggcctggccgtgaaaaccattcagcagcacctggggcagctgaatatgctgcacagaagaagcggactgcccagacctagcgatagcaacgccgtgtctctggtcatgcggcggatcagaaaagaaaacgtggacgccggcgagagagccaaacaggccctggcctttgagagaaccgacttcgatcaagtgcggtccctgatggaaaacagcgacaggtgccaggatatccgcaatctggctttcctgggaatcgcctacaacaccctgctgcggattgccgagatcgcccggatcagagtgaaggacatcagcagaacagacggcggcaggatgctgatccacatcggcagaactaagaccctggtgtccaccgcaggcgtggaaaaagctctgtctctgggcgtgaccaagctggtggaaagatggatctccgtgtccggcgtggccgacgatcccaacaattacctgttctgcagagtgcgcaagaacggcgtggcagccccttctgctacaagccagctgagcacaagagccctggaaggcatcttcgaggccacacacagactgatctacggcgccaaggacgacagcggccagagatatcttgcttggagcggacactccgccagagtgggagctgctagagatatggctagagccggcgtgtcaatccccgagattatgcaagctggcggctggaccaacgtgaacatcgtgatgaattacattcggaacctcgacagcgaaaccggcgccatggttcgactgctggaagatggcgat

SEQ ID NO: 11 - DNA, 951 bp, synthetic construct
  source 1..951: mol_type = other DNA
atgttccaagccgccgagaggcctcaagaatgggccatggaaggccctagagatggcctgaagaaagagcggctgctggacgacagacacgatgccggactggacgccatgaaggacgaggaatacgagcagatggtcaaagagctgcaagagatccggctggaaccccaagaagtgcctagaggctctgagccctggaagcagcagctgacagaggacggcgatagctttctgcacctggccatcatccacgaagagaaggccctgaccatggaagtgatccggcaagtgaaaggcgacctggccttcctgaacttccagaacaacctgcagcagacccctctgcatctggccgtgatcaccaaccagcctgagattgccgaagctctgctcggagccggatgtgaccctgagctgagagacttcagaggcaacacccctctccacctggcttgcgaacaaggctgtctggcttctgtgggcgtgctgacccagagctgtaccacacctcatctgcacagcatcctgaaggccaccaactacaacggccacacatgtctgcacctcgcctccattcacggctacctgggaatcgtggaactgctggtgtctctgggcgccgacgtgaacgctcaagagccttgcaatggcagaacagccctgcaccttgccgtggacctgcagaatcctgatctggtgtccctgctgctgaagtgcggcgctgatgtgaacagagtgacctaccagggctacagcccctaccagcttacatggggcagacccagcaccagaatccagcagcaactgggccagctgaccctggaaaacctgcagatgctgcccgagagcgaggacgaagagagctacgataccgagtccgagttcacagagttcaccgaggacgagctgccctacgacgattgtgtgtttggcggccagagactgaccctg

SEQ ID NO: 12 - DNA, 4227 bp, synthetic construct
  source 1..4227: mol_type = other DNA
atggacgtccaccaccaccatcatcacatggcccccaagaagaagagaaaggtgggcatccacggcgtgcctgccgccgacaagaagtactccatcggcctggacattggcaccaacagcgtgggctgggccgtgattaccgacgagtataaggtgcccagcaagaagttcaaagtgctgggaaacaccgacagacacagcattaagaagaatctgatcggcgccctcctgttcgacagcggcgaaaccgccgaggccaccagactgaagcggaccgccagaagacggtacaccagaagaaagaacagaatctgctacctgcaggagatcttcagcaacgagatggccaaggtggatgactccttcttccacagactggaggagtccttcctggtggaggaggacaaaaagcacgagagacaccccatcttcggaaacatcgtggatgaggtggcctaccacgagaaataccccaccatctaccacctgagaaagaagctggtggacagcaccgacaaggctgacctgagactgatctacctggccctggcccatatgatcaagttcagaggccacttcctgattgagggcgacctgaaccccgacaacagcgacgtggacaagctgttcatccagctggtgcagacctacaaccagctgtttgaggagaaccccatcaacgctagcggcgtggacgccaaggccattctgtccgccaggctgagcaaaagcaggaggctggagaatctcatcgcccagctgcctggcgaaaagaagaacggcctgttcggcaacctgatcgctctgagcctgggcctgacccccaacttcaaaagcaacttcgacctggccgaagacgccaaactgcagctgtccaaggacacctacgacgacgacctggacaacctgctggcccagatcggcgatcagtacgccgacctgttcctggccgccaagaacttgagcgacgctatcctgctgagcgacatcctcagagtgaacaccgagatcaccaaggcccccctgagcgccagcatgatcaaaagatacgacgagcaccatcaggacctgaccctgctgaaggccctggtgaaacagcagctgcccgagaagtacaaggaaatcttcttcgaccagagcaagaatggctacgccggctacatcgacggcggcgccagccaggaggagttctacaagttcatcaagcccatcctggaaaagatggacggcaccgaggagctgctggtgaagctgaacagagaggatctgctcagaaagcagagaaccttcgacaacggcagcatcccccaccagatccacctgggcgagctgcacgccatcctgagaaggcaggaagacttctaccccttcctgaaggacaatagagagaagattgagaagatcctgaccttcagaatcccctactacgtgggccccctggccagaggcaactccagattcgcctggatgaccagaaagagcgaggaaaccatcaccccctggaacttcgaggaggtggtcgacaagggcgcctccgcccagagcttcatcgagagaatgaccaactttgacaagaacctgcccaacgagaaggtgctgcccaagcacagcctgctgtacgagtatttcaccgtgtacaacgagctgaccaaggtgaagtacgtgaccgagggcatgagaaagcccgccttcctgtccggcgagcagaagaaggcaatcgtggacctgctgttcaagaccaataggaaggtgacagtgaagcagctgaaggaggactacttcaagaagatcgagtgcttcgactccgtggagatctctggcgtggaagacagattcaacgccagcctgggaacctaccatgatctgctgaaaatcatcaaggacaaggacttcctggataacgaagagaacgaagatatcctggaggacatcgtgctgactctgacactgttcgaggacagagagatgatcgaggagaggctgaagacctacgcccacctgtttgacgacaaggtgatgaagcaactgaagagaaggagatacaccggctggggcagactgagcaggaagctgatcaacggcatcagagacaagcagagcggcaagaccatcctcgacttcctcaagagcgacggcttcgccaacagaaacttcatgcagctgatccacgacgacagcctgacattcaaggaagacatccagaaggctcaggtgagcggacagggcgatagcctgcatgagcatattgccaatctggccggaagccctgccatcaagaagggcatcctgcagaccgtgaaggtggtggacgagctcgtgaaggtgatgggcagacacaagcccgagaacatcgtgatcgagatggccagggagaaccagaccacccagaagggacagaagaacagcagagagagaatgaagagaatcgaggagggcatcaaggagctgggcagccagatcttgaaggagcaccccgtggagaacacacagctccagaacgagaaactgtacctgtactacctgcagaacggccgggacatgtacgtggaccaggagcttgacatcaacagactgagcgattatgatgtggaccacatcgtgccccagagctttctgaaggacgacagcatcgacaacaaggtgctgaccagatccgacaagaacagaggcaaaagcgacaacgtgcccagcgaggaggtggtgaagaagatgaagaactactggaggcagctgctgaacgctaagctcatcacccagagaaagttcgacaacctgaccaaggctgagaggggcggcctgagcgagctggacaaggccggattcatcaagagacagctggtggaaaccagacagatcaccaagcacgtggcccagatcctggactccagaatgaacaccaagtacgacgagaacgacaagctgatcagagaagtgaaagtgatcacactgaagtccaagctggtgagcgacttcaggaaggacttccagttctacaaggtgagagagatcaacaactaccaccacgcccacgacgcctacctgaacgccgtggtgggcaccgccctgatcaaaaagtaccctaagctggaaagcgagttcgtgtacggcgactacaaagtgtacgacgtgaggaagatgatcgccaagagcgagcaggagatcggcaaggccaccgccaagtatttcttctacagcaacatcatgaacttcttcaagaccgagatcactctggccaacggggagatcaggaagaggcccctgatcgaaaccaacggcgaaaccggcgagatcgtgtgggacaagggcagagacttcgccaccgtgagaaaggtgctgtccatgccccaggtgaatatcgtgaagaaaaccgaggtgcagaccggcggattcagcaaggagagcatcctgcccaagagaaacagggacaaactgatcgctagaaagaaggactgggaccccaagaagtacggcggtttcgacagccccaccgtggcctatagcgtgctggtggtggccaaggtggagaaaggcaagagcaaaaagctgaagagcgtgaaagagctgctgggcatcaccatcatggagagaagctccttcgagaagaaccccatcgacttcctggaggccaagggctacaaggaggtgaaaaaggacctgatcatcaagctgcctaagtacagcctgtttgagctggagaacggcagaaagagaatgctggcctccgccggcgagctgcagaagggcaacgagctggccctgcctagcaagtacgtgaacttcctgtatctggccagccactacgagaagctgaaaggcagccccgaggacaacgagcagaagcagctgttcgtggagcagcacaagcactacctggacgagatcatcgagcagatctccgagttctccaagagggtgatcctggccgacgccaacctggacaaagtgctgagcgcctacaacaagcacagagacaagcccatcagagagcaggccgagaatatcatccacctgttcaccctgacaaacctgggcgcccctgccgccttcaagtacttcgacaccaccatcgaccggaagagatacacctccaccaaagaggtgctggacgccaccctgatccaccagagcatcaccggcctgtacgagacaagaatcgatctgagccagctgggcggcgataaaagaccagccgccaccaagaaggccggccaggccaagaagaagaaa

SEQ ID NO: 13 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 14 - protein, 523 aa, synthetic construct
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRKVSGWRLFKKIS

SEQ ID NO: 15 - protein, 988 aa, synthetic construct
MAPPMTLQQWIIWKKMNKAHEALQNTTTVTEQQKEQIILDIQNEEVQPTRRDKFRYLLYTCCATSSRVLAWMFLVCILLIIVLVSCFVTISRIQWNKDIQVLGPVIDWNVTQRAVYQPLQTRRIARSLRMQHPVPKYVEVNMTSIPQGVYYEPHPEPIVVKERVLGLSQILMINSENIANNANLTQEVKKLLTEMVNEEMQSLSDVMIDFEIPLGDPRDQEQYIHRKCYQEFANCYLVKYKEPKPWPKEGLIADQCPLPGYHAGLTYNRQSIWDYYIKVESIRPANWTTKSKYGQARLGSFYIPSSLRQINVSHVLFCSDQLYSKWYNIENTIEQNERFLLNKLNNLTSGTSVLKKRALPKDWSSQGKNALFREINVLDICSKPESVILLNTSYYSFSLWEGDCNFTKDMISQLVPECDGFYNNSKWMHMHPYACRFWRSKNEKEETKCRDGETKRCLYYPLWDSPESTYDFGYLAYQKNFPSPICIEQQKIRDQDYEVYSLYQECKIASKAYGIDTVLFSLKNFLNYTGTPVNEMPNARAFVGLIDPKFPPSYPNVTREHYTSCNNRKRRSVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTKKKNQ

SEQ ID NO: 16 - protein, 238 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 17 - protein, 228 aa, synthetic construct
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 18 - protein, 236 aa, synthetic construct
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 19 - protein, 879 aa, synthetic construct
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 20 - protein, 168 aa, synthetic construct
ALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHN

SEQ ID NO: 21 - protein, 34 aa, synthetic construct
GSGYIPEAPRDGQAYVRKDGEWVLLSTFLSPANR

SEQ ID NO: 22 - protein, 343 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 23 - protein, 317 aa, synthetic construct
MFQAAERPQEWAMEGPRDGLKKERLLDDRHDAGLDAMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 24 - protein, 1409 aa, synthetic construct
MDVHHHHHHMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVKQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNRDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK